(19) United States
(12) Patent Application Publication
(10) Pub. No.: US 2024/0150736 A1
(43) Pub. Date: May 9, 2024

(54) FUSION PROTEINS COMPRISING SULFOGLUCOSAMINE SULFOHYDROLASE ENZYMES AND METHODS THEREOF

(71) Applicant: DENALI THERAPEUTICS INC., South San Francisco, CA (US)

(72) Inventors: Tina GIESE, South San Francisco, CA (US); Gunasekaran KANNAN, Daly City, CA (US); Mihalis S. KARIOLIS, San Mateo, CA (US); Cathal S. MAHON, San Francisco, CA (US)

(73) Assignee: DENALI THERAPEUTICS INC., South San Francisco, CA (US)

(21) Appl. No.: 18/411,592

(22) Filed: Jan. 12, 2024

Related U.S. Application Data
(62) Division of application No. 17/855,543, filed on Jun. 30, 2022, now Pat. No. 11,884,944.
Application No. 17/855,543 is a continuation of application No. PCT/US2021/054860, filed on Oct. 13, 2021.
(60) Provisional application No. 63/091,800, filed on Oct. 14, 2020.

(51) Int. Cl. C12N 9/14; A61K 38/46; A61P 3/00

(52) U.S. Cl. CPC C12N 9/14; A61K 38/46; A61P 3/00; C12Y 310/01001; C07K 2319/30

(57) ABSTRACT

Provided herein are proteins, which are capable of being transported across the blood-brain barrier (BBB) and comprise sulfoglucosamine sulfohydrolase (SGSH) enzyme-Fc fusion polypeptides. Certain embodiments also provide methods of using such proteins to treat Sanfilippo syndrome A.

### CROSS-REFERENCE TO RELATED APPLICATIONS

This application is a divisional of U.S. patent application Ser. No. 17/855,543, filed Jun. 30, 2022, which is a continuation of International Application Serial No. PCT/US2021/054860, filed Oct. 13, 2021, which claims priority to U.S. Provisional Application Ser. No. 63/091,800, filed Oct. 14, 2020. The entire content of the applications referenced above are hereby incorporated by reference herein.

### SEQUENCE LISTING

The instant application contains a Sequence Listing which has been submitted electronically in XML file format and is hereby incorporated by reference in its entirety. Said XML copy, created on Jan. 3, 2024, is named 02900_027US2_SL.xml and is 218,105 bytes in size.

### BACKGROUND

Sanfilippo syndrome, or MPS III, is a rare, neurodegenerative disorder that results from certain defects in lysosomal function. The most common type of Sanfilippo syndrome is type A, which is caused by genetic mutations in the SGSH gene. Insufficient N-sulfoglucosamine sulfohydrolase (SGSH) activity leads to accumulation of heparan sulfate-derived oligosaccharides and to lysosomal dysfunction in multiple organs and tissues, particularly the brain and spinal cord. Treatments for Sanfilippo syndrome remain largely supportive; while the deficient enzyme may be administered intravenously, it has little effect on the brain due to difficulties in delivering the recombinant enzyme across the blood-brain barrier (BBB). Accordingly, there is a need for more effective therapies that treat both the peripheral and central nervous system (CNS) symptoms of Sanfilippo syndrome A.

### SUMMARY

Thus, provided herein is a specific enzyme replacement therapy, which has the capability of crossing the BBB and treating both the peripheral and CNS manifestations of Sanfilippo syndrome A. In particular, certain embodiments provide a protein comprising: (a) a first Fc polypeptide linked to a first N-sulfoglucosamine sulfohydrolase (SGSH) amino acid sequence, an SGSH variant amino acid sequence, or a catalytically active fragment thereof; and (b) a second Fc polypeptide linked to a second SGSH amino acid sequence, an SGSH variant amino acid sequence, or a catalytically active fragment thereof, wherein the second Fc polypeptide comprises a sequence having at least 80% identity to SEQ ID NO: 37, and having Ala at position 389, according to EU numbering. In some embodiments, the second Fc polypeptide comprises at the following positions, according to EU numbering: Glu at position 380; Ala at position 389; and Asn at position 390. In some embodiments, the second Fc polypeptide comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In certain embodiments, the second Fc polypeptide specifically binds to a transferrin receptor (TfR) or is capable of specifically binding to a TfR. In certain embodiments, the second Fc polypeptide binds to the apical domain of the TfR. In certain embodiments, the binding of the protein to the TfR does not substantially inhibit binding of transferrin to the TfR. In certain embodiments, the protein binds to a TfR with an affinity of from about 100 nM to about 500 nM, or optionally from about 150 nM to about 400 nM. In certain embodiments, the protein is capable of being transported across the blood-brain barrier of a subject.

In certain embodiments, the first SGSH amino acid sequence comprises an amino acid sequence having at least 80%, 85%, 90%, or 95% identity to any one of SEQ ID NOS:58-60. In certain embodiments, the first SGSH amino acid sequence comprises the amino acid sequence of any one of SEQ ID NOS:58-60.

In certain embodiments, the second SGSH amino acid sequence comprises an amino acid sequence having at least 80%, 85%, 90%, or 95% identity to any one of SEQ ID NOS:58-60. In certain embodiments, the second SGSH amino acid sequence comprises the amino acid sequence of any one of SEQ ID NOS:58-60.

In certain embodiments, the first Fc polypeptide is linked to the first SGSH amino acid sequence, SGSH variant amino acid sequence, or a catalytically active fragment thereof by a peptide bond or by a polypeptide linker. In certain embodiments, the second Fc polypeptide is linked to the second SGSH amino acid sequence, SGSH variant amino acid sequence, or a catalytically active fragment thereof by a peptide bond or by a polypeptide linker. In certain embodiments, the polypeptide linker is a flexible polypeptide linker. In certain embodiments, the flexible polypeptide linker is a glycine-rich linker. In certain embodiments, the polypeptide linker is GS, G₄S (SEQ ID NO:8) or (G₄S)₂ (SEQ ID NO:9).

In certain embodiments, the N-terminus of the first Fc polypeptide is linked to the first SGSH amino acid sequence, SGSH variant amino acid sequence, or a catalytically active fragment thereof. In certain embodiments, the C-terminus of the first Fc polypeptide is linked to the first SGSH amino acid sequence, SGSH variant amino acid sequence, or a catalytically active fragment thereof. In certain embodiments, the N-terminus of the second Fc polypeptide is linked to the second SGSH amino acid sequence, SGSH variant amino acid sequence, or a catalytically active fragment thereof. In certain embodiments, the C-terminus of the second Fc polypeptide is linked to the second SGSH amino acid sequence, SGSH variant amino acid sequence, or a catalytically active fragment thereof. In certain embodiments, the N-terminus of the first Fc polypeptide is linked to the first SGSH amino acid sequence, SGSH variant amino acid sequence, or a catalytically active fragment thereof, and the N-terminus of the second Fc polypeptide is linked to the second SGSH amino acid sequence, SGSH variant amino acid sequence, or a catalytically active fragment thereof. In certain embodiments, the C-terminus of the first Fc polypeptide is linked to the first SGSH amino acid sequence, SGSH variant amino acid sequence, or a catalytically active fragment thereof, and the C-terminus of the second Fc polypeptide is linked to the second SGSH amino acid sequence, SGSH variant amino acid sequence, or a catalytically active fragment thereof.

In certain embodiments, the first Fc polypeptide and the second Fc polypeptide each contain modifications that promote heterodimerization. In certain embodiments, one of the Fc polypeptides has a T366W substitution and the other Fc polypeptide has T366S, L368A, and Y407V substitutions, according to EU numbering.

In certain embodiments, the first Fc polypeptide contains the T366S, L368A, and Y407V substitutions and the second Fc polypeptide contains the T366W substitution. In certain embodiments, the first Fc polypeptide comprises an amino acid sequence having at least 80%, 85%, 90%, 95% or 100% identity to any one of SEQ ID NOS: 12-19 and 28-31; and the second Fc polypeptide comprises an amino acid sequence having at least 80%, 85%, 90%, 95% or 100% identity to any one of SEQ ID NOS: 34-41 and 54-57.

In certain embodiments, the first Fc polypeptide contains the T366W substitution and the second Fc polypeptide contains the T366S, L368A, and Y407V substitutions. In certain embodiments, the first Fc polypeptide comprises an amino acid sequence having at least 80%, 85%, 90%, 95% or 100% identity to any one of SEQ ID NOS: 24-27; and the second Fc polypeptide comprises an amino acid sequence having at least 80%, 85%, 90%, 95% or 100% identity to any one of SEQ ID NOS: 48-53.

In certain embodiments, the first Fc polypeptide and/or the second Fc polypeptide comprises a native FcRn binding site.

In certain embodiments, the first Fc polypeptide and the second Fc polypeptide do not have effector function. In certain embodiments, the first Fc polypeptide and/or the second Fc polypeptide includes a modification that reduces effector function. In certain embodiments, the modification that reduces effector function is the substitutions of Ala at position 234 and Ala at position 235, according to EU numbering.

In certain embodiments, the first Fc polypeptide comprises an amino acid sequence having at least 80%, 85%, 90%, 95% or 100% identity to any one of SEQ ID NOS: 14-19 and 26-31. In certain embodiments, the first Fc polypeptide comprises an amino acid sequence having at least 80%, 85%, 90%, 95% or 100% identity to any one of SEQ ID NOS: 14, 15, 28, and 29. In certain embodiments, the first Fc polypeptide comprises an amino acid sequence having at least 80%, 85%, 90%, 95% or 100% identity to any one of SEQ ID NOS:18, 19, 30, and 31. In certain embodiments, the first Fc polypeptide linked to the first SGSH amino acid sequence comprises an amino acid sequence having at least 80%, 85%, 90%, 95% or 100% identity to any one of SEQ ID NOS: 61-88, and 117-118. In certain embodiments, the first Fc polypeptide linked to the first SGSH amino acid sequence comprises an amino acid sequence having at least 80%, 85%, 90%, 95% or 100% identity to any one of SEQ ID NOS: 61-68, 73-76, 81-84, and 117-118.

In certain embodiments, the second Fc polypeptide comprises an amino acid sequence having at least 80%, 85%, 90%, 95% or 100% identity to any one of SEQ ID NOS: 36-41 and 50-57. In certain embodiments, the second Fc polypeptide comprises an amino acid sequence having at least 80%, 85%, 90%, 95% or 100% identity to any one of SEQ ID NOS: 36, 37, 54, and 55. In certain embodiments, the second Fc polypeptide comprises an amino acid sequence having at least 80%, 85%, 90%, 95% or 100% identity to any one of SEQ ID NOS:40 41, 56, and 57. In certain embodiments, the second Fc polypeptide linked to the second SGSH amino acid sequence comprises an amino acid sequence having at least 80%, 85%, 90%, 95% or 100% identity to any one of SEQ ID NOS: 89-116, and 119-120. In certain embodiments, the second Fc polypeptide linked to the second SGSH amino acid sequence comprises an amino acid sequence having at least 80%, 85%, 90%, 95% or 100% identity to any one of SEQ ID NOS: 89-96, 101-104, 109-112, and 119-120.

In certain embodiments, the first Fc polypeptide and/or the second Fc polypeptide comprises amino acid changes relative to the native Fc sequence that extend serum half-life. In certain embodiments, the amino acid changes comprise substitutions of Tyr at position 252, Thr at position 254, and Glu at position 256, according to EU numbering. In certain embodiments, the amino acid changes comprise substitutions of Leu at position 428 and Ser at position 434, according to EU numbering. In certain embodiments, the amino acid changes comprise a substitution of Ser or Ala at position 434, according to EU numbering.

In certain embodiments, the first Fc polypeptide linked to the first SGSH amino acid sequence comprises an amino acid sequence of any one of SEQ ID NOS: 61-68, 73-76, and 81-84; and the second Fc polypeptide linked to the second SGSH amino acid sequence comprises an amino acid sequence of any one of SEQ ID NOS: 89-96, 101-104, and 109-112.

In certain embodiments, the first Fc polypeptide linked to the first SGSH amino acid sequence comprises an amino acid sequence of any one of SEQ ID NOS: 61-64; and the second Fc polypeptide linked to the second SGSH amino acid sequence comprises an amino acid sequence of any one of SEQ ID NOS: 89-92. In certain embodiments, the first Fc polypeptide linked to the first SGSH amino acid sequence comprises an amino acid sequence of SEQ ID NOS: 63 or 64; and the second Fc polypeptide linked to the second SGSH amino acid sequence comprises an amino acid sequence of SEQ ID NOS: 91 or 92.

In certain embodiments, the first Fc polypeptide linked to the first SGSH amino acid sequence comprises an amino acid sequence of SEQ ID NOS: 75 or 76; and the second Fc polypeptide linked to the second SGSH amino acid sequence comprises an amino acid sequence of SEQ ID NOS: 103 or 104.

In certain embodiments, the first Fc polypeptide linked to the first SGSH amino acid sequence comprises an amino acid sequence of SEQ ID NOS: 83 or 84; and the second Fc polypeptide linked to the second SGSH amino acid sequence comprises an amino acid sequence of SEQ ID NOS: 111 or 112.

In certain embodiments, the first Fc polypeptide linked to the first SGSH amino acid sequence comprises an amino acid sequence of any one of SEQ ID NOS: 65-68; and the second Fc polypeptide linked to the second SGSH amino acid sequence comprises an amino acid sequence of any one of SEQ ID NOS: 93-96. In certain embodiments, the first Fc polypeptide linked to the first SGSH amino acid sequence comprises an amino acid sequence of SEQ ID NOS: 67 or 68; and the second Fc polypeptide linked to the second SGSH amino acid sequence comprises an amino acid sequence of SEQ ID NOS: 95 or 96.

In certain embodiments, the first Fc polypeptide linked to the first SGSH amino acid sequence comprises an amino acid sequence of SEQ ID NO: 118; and the second Fc polypeptide linked to the second SGSH amino acid sequence comprises an amino acid sequence of SEQ ID NO: 120.

In certain embodiments, uptake of the SGSH amino acid sequence into the brain is at least ten-fold greater as compared to the uptake of the SGSH amino acid sequence in the absence of the first Fc polypeptide and the second Fc polypeptide or as compared to the uptake of the SGSH enzyme without the modifications to the second Fc polypeptide that result in TfR binding.

In certain embodiments, the first Fc polypeptide is not modified to bind to a blood-brain barrier (BBB) receptor and the second Fc polypeptide is modified to specifically bind to a TfR.

In certain embodiments, the protein does not include an immunoglobulin heavy and/or light chain variable region sequence or an antigen-binding portion thereof.

Certain embodiments also provide, a polypeptide comprising a Fc polypeptide linked to an SGSH amino acid sequence, an SGSH variant amino acid sequence, or a catalytically active fragment thereof, wherein the Fc polypeptide i) comprises a sequence having at least 90% identity to SEQ ID NO: 37; ii) has one or more modifications that promote its heterodimerization to another Fc polypeptide; and iii) has Ala at position 389, according to EU numbering. In some embodiments, the Fc polypeptide comprises at the following positions, according to EU numbering: Glu at position 380; Ala at position 389; and Asn at position 390. In some embodiments, the Fc polypeptide comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In certain embodiments, the Fc polypeptide specifically binds to a transferrin receptor (TfR) or is capable of specifically binding to a TfR. In certain embodiments, the Fc polypeptide is linked to the SGSH amino acid sequence, SGSH variant amino acid sequence, or catalytically active fragment thereof by a peptide bond or by a polypeptide linker. In certain embodiments, the polypeptide is a fusion polypeptide comprising from N- to C-terminus: the SGSH amino acid sequence, SGSH variant amino acid sequence, or catalytically active fragment; a polypeptide linker; and the Fc polypeptide. In certain embodiments, the polypeptide is a fusion polypeptide comprising from N- to C-terminus: the Fc polypeptide; a polypeptide linker; and the SGSH amino acid sequence, SGSH variant amino acid sequence, or catalytically active fragment.

In certain embodiments, the Fc polypeptide contains T366S, L368A, and Y407V substitutions, according to EU numbering. In certain embodiments, the polypeptide comprises an amino acid sequence having at least 80%, 85%, 90%, 95% or 100% identity to any one of SEQ ID NOS:97-100, 105-108, and 113-116.

In certain embodiments, the Fc polypeptide contains a T366W substitution. In certain embodiments, the polypeptide comprises an amino acid sequence having at least 80%, 85%, 90%, 95% or 100% identity to any one of SEQ ID NOS:89-96, 101-104, 109-112, and 119-120.

In certain embodiments, a protein comprises the Fc polypeptide, wherein the Fc polypeptide is dimerized to the other Fc polypeptide. Thus, certain embodiments provide a protein comprising a Fc polypeptide as described herein and the other Fc polypeptide.

Certain embodiments provide a polynucleotide comprising a nucleic acid sequence encoding a polypeptide as described herein. Certain embodiments also provide a vector comprising a polynucleotide as described herein. Certain embodiment provide a host cell comprising a polynucleotide as described herein or a vector as described herein. In certain embodiments, such a host cell further comprises a polynucleotide comprising a nucleic acid sequence encoding the other Fc polypeptide.

Provided herein is a method of making a protein or polypeptide as described herein.

Certain embodiments provide a method for producing a polypeptide comprising an Fc polypeptide that is linked to an SGSH amino acid sequence, SGSH variant amino acid sequence, or catalytically active fragment, comprising culturing a host cell under conditions in which the polypeptide encoded by a polynucleotide as described herein is expressed.

Certain embodiments provide a pair of polynucleotides comprising a first nucleic acid sequence encoding a first Fc polypeptide linked to a first SGSH amino acid sequence, SGSH variant amino acid sequence, or a catalytically active fragment thereof; and a second nucleic acid sequence encoding a second Fc polypeptide linked to a second SGSH amino acid sequence, SGSH variant amino acid sequence, or a catalytically active fragment thereof. Certain embodiments also provide one or more vectors comprising a pair of polynucleotides as described herein. Certain embodiments provide a host cell comprising a pair of polynucleotides as described herein, or one or more vectors as described herein.

Certain embodiments also provide a method for producing a protein comprising a first Fc polypeptide linked to a first SGSH amino acid sequence, SGSH variant amino acid sequence, or a catalytically active fragment thereof, and a second Fc polypeptide linked to a second SGSH amino acid sequence, SGSH variant amino acid sequence, or a catalytically active fragment thereof, comprising culturing a host cell under conditions in which a pair of polynucleotides as described herein are expressed.

Certain embodiments provide a pharmaceutical composition comprising a protein or polypeptide as described herein and a pharmaceutically acceptable carrier and/or excipient.

Certain embodiments provide a method of treating Sanfilippo syndrome A, the method comprising administering a protein as described herein or a polypeptide as described herein to a patient in need thereof.

Certain embodiments provide a protein as described herein or a polypeptide as described herein for use in treating Sanfilippo syndrome A in a patient in need thereof.

Certain embodiments provide the use of a protein as described herein or a polypeptide as described herein in the preparation of a medicament for treating Sanfilippo syndrome A in a patient in need thereof.

Certain embodiments provide a method of decreasing the accumulation of a toxic metabolic product in a patient having Sanfilippo syndrome A, the method comprising administering a protein as described herein or a polypeptide as described herein to the patient.

Certain embodiments provide a protein as described herein or a polypeptide as described herein for use in decreasing the accumulation of a toxic metabolic product in a patient having Sanfilippo syndrome A.

Certain embodiments provide the use of a protein as described herein or a polypeptide as described herein in the preparation of a medicament for decreasing the accumulation of a toxic metabolic product in a patient having Sanfilippo syndrome A.

In certain embodiments, the toxic metabolic product comprises heparan sulfate-derived oligosaccharides.

### BRIEF DESCRIPTION OF THE FIGURES

FIGS. 1A-1C. Illustration of exemplary ETV:SGSH fusion proteins having varying linker lengths between the SGSH enzyme and the Fc polypeptide hinge region.

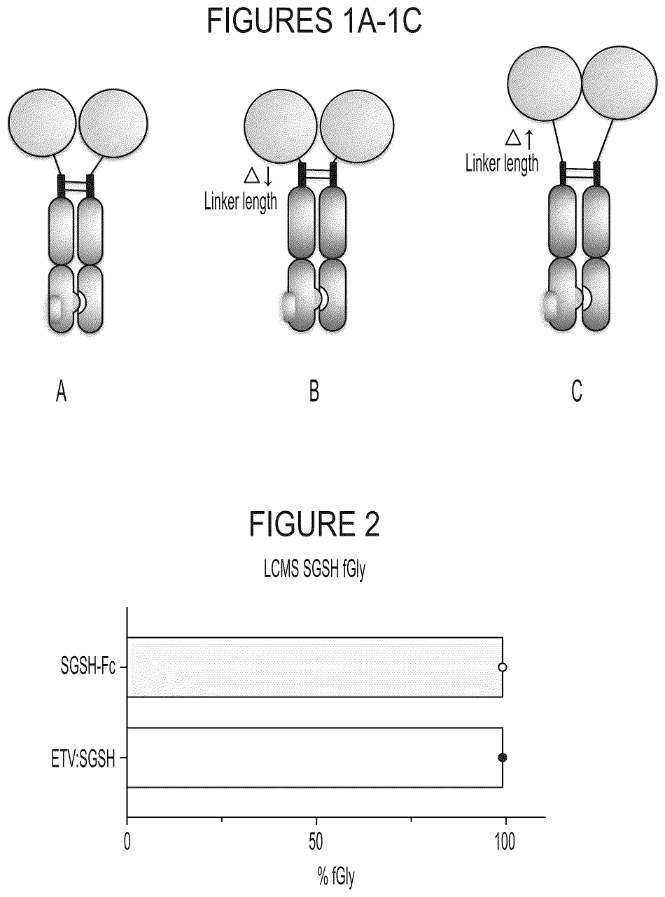

FIG. 2. fGly content of SGSH-Fc and ETV:SGSH fusion proteins as determined by LCMS.

FIG. 3. In vitro evaluation of enzymatic activity of SGSH-Fc and ETV:SGSH fusion proteins.

FIG. 4. Evaluation of cellular activity of SGSH-Fc fusion proteins in fibroblasts from MPSIIIA patients and healthy controls using a ³⁵S pulse-chase assay.

FIG. 5. Serum concentration of SGSH-Fc and ETV:SGSH fusion proteins.

FIGS. 6A-6B. Liver concentration of SGSH-Fc and ETV:SGSH fusion proteins at 2 hours (FIG. 6A) and 8 hours (FIG. 6B).

FIG. 7. Brain concentration of SGSH-Fc and ETV:SGSH fusion proteins.

FIG. 8. Total heparan sulfate levels in liver.

FIG. 9. Total heparan sulfate levels in brain.

FIG. 10. Total heparan sulfate levels in CSF.

FIG. 11. Total heparan sulfate levels in brain after administration of two different ETV:SGSH fusion proteins.

### DETAILED DESCRIPTION

There is currently a need for new therapeutics for the treatment of Sanfilippo syndrome A, specifically therapeutics that treat the neurocognitive phenotype. Described herein is a specific enzyme replacement therapy termed ETV:SGSH, which has the capability of crossing the BBB and treating both the peripheral and CNS manifestations of Sanfilippo syndrome A. As used herein, the term “ETV:SGSH” refers to a dimeric protein that is capable of being transported across the BBB and comprises a first Fc polypeptide and a second Fc polypeptide, which are each linked (e.g., fused) to an SGSH enzyme, an SGSH enzyme variant, or a catalytically active fragment thereof. As discussed in the Examples, a murine mouse model of Sanfilippo syndrome A showed a greater than 50% reduction in brain glycosaminoglycans (GAGs) and a greater than 80% reduction in CSF GAGs following a single intravenous dose of ETV:SGSH.

### Protein Molecules Comprising SGSH Enzyme-Fc Fusion Polypeptides

As described herein, certain embodiments provide a protein molecule comprising an SGSH enzyme-Fc fusion polypeptide. An SGSH enzyme incorporated into the protein is catalytically active, i.e., it retains the enzymatic activity. In some aspects, a protein described herein comprises: (i) an Fc polypeptide, which may contain modifications (e.g., one or more modifications that promote heterodimerization) or may be a wild-type Fc polypeptide; and an SGSH enzyme; and (ii) an Fc polypeptide, which contains modifications that result in binding to a blood-brain barrier (BBB) receptor, e.g., a transferrin receptor (TfR), and optionally one or more additional modifications (e.g., one or more modifications that promote heterodimerization); and an SGSH enzyme.

In some embodiments, a protein as described herein comprises a catalytically active fragment or variant of a wild-type SGSH. In some embodiments, the SGSH enzyme is a variant or a catalytically active fragment of an SGSH protein that comprises the amino acid sequence of any one of SEQ ID NOS:58, 59 and 60. In some embodiments, a catalytically active variant or fragment of an SGSH enzyme has at least 50%, at least 55%, at least 60%, at least 65%, at least 70%, at least 75%, at least 80%, at least 85%, at least 90%, at least 95%, or greater of the activity of the wild-type SGSH enzyme.

In some embodiments, an SGSH enzyme, or a catalytically active variant or fragment thereof, that is present in a protein described herein, retains at least 25% of its activity compared to its activity when not joined to an Fc polypeptide or a TfR-binding Fc polypeptide. In some embodiments, an SGSH enzyme, or a catalytically active variant or fragment thereof, retains at least 10%, or at least 15%, 20%, 25%, 30%, 35%, 40%, 45%, 50%, 55%, 60%, 65%, 70%, 75%, 80%, 85%, 90%, or 95%, of its activity compared to its activity when not joined to an Fc polypeptide or a TfR-binding Fc polypeptide. In some embodiments, an SGSH enzyme, or a catalytically active variant or fragment thereof, retains at least 80%, 85%, 90%, or 95% of its activity compared to its activity when not joined to an Fc polypeptide or a TfR-binding Fc polypeptide. In some embodiments, fusion to an Fc polypeptide does not decrease the activity of the SGSH enzyme, or catalytically active variant or fragment thereof. In some embodiments, fusion to a TfR-binding Fc polypeptide does not decrease the activity of the SGSH enzyme.

### Fc Polypeptide Modifications

An Fc polypeptide incorporated in a fusion protein described herein may comprise certain modifications. For example, an Fc polypeptide may comprise modifications that result in binding to a blood-brain barrier (BBB) receptor, e.g., a transferrin receptor (TfR). Additionally, an Fc polypeptide may comprise other modifications, such as modifications that promote heterodimerization, increase serum stability or serum half-life, modulate effector function, influence glycosylation, and/or reduce immunogenicity in humans. Thus, in certain embodiments, a fusion protein described herein comprises two Fc polypeptides, wherein one Fc is a wild-type Fc polypeptide, e.g., a human IgG1 Fc polypeptide; and the other Fc is modified to bind to a blood-brain barrier (BBB) receptor, e.g., transferrin receptor (TfR), and optionally further comprises one or more additional modifications. In certain other embodiments, both Fc polypeptides each comprise independently selected modifications (e.g., a modification described herein).

Amino acid residues designated in various Fc modifications, including those introduced in a modified Fc polypeptide that binds to a BBB receptor, e.g., TfR, are numbered herein using EU index numbering. Any Fc polypeptide, e.g., an IgG1, IgG2, IgG3, or IgG4 Fc polypeptide, may have modifications, e.g., amino acid substitutions, in one or more positions as described herein.

A modified (e.g., enhancing heterodimerization and/or BBB receptor-binding) Fc polypeptide present in a fusion protein described herein can have at least 70% identity, at least 75% identity, at least 80% identity, at least 85% identity, at least 90% identity, at least 95% identity, at least 96% identity, at least 97% identity, at least 98% identity, or at least 99% identity to a native Fc region sequence or a fragment thereof, e.g., a fragment of at least 50 amino acids or at least 100 amino acids, or greater in length. In some embodiments, the native Fc amino acid sequence is the Fc region sequence of SEQ ID NO:1. In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to amino acids 1-110 of SEQ ID NO:1, or to amino acids 111-217 of SEQ ID NO:1, or a fragment thereof, e.g., a fragment of at least 50 amino acids or at least 100 amino acids, or greater in length.

In some embodiments, a modified (e.g., enhancing heterodimerization and/or BBB receptor-binding) Fc polypeptide comprises at least 50 amino acids, or at least 60, 65, 70, 75, 80, 85, 90, or 95 or more, or at least 100 amino acids, or more, that correspond to a native Fc region amino acid sequence. In some embodiments, the modified Fc polypeptide comprises at least 25 contiguous amino acids, or at least 30, 35, 40, or 45 contiguous amino acids, or 50 contiguous amino acids, or at least 60, 65, 70, 75, 80 85, 90, or 95 or more contiguous amino acids, or 100 or more contiguous amino acids, that correspond to a native Fc region amino acid sequence, such as SEQ ID NO:1.

### Modifications for Blood-Brain Barrier (BBB) Receptor Binding

In some aspects, provided herein are fusion proteins that are capable of being transported across the blood-brain barrier (BBB). Such a protein comprises a modified Fc polypeptide that binds to a BBB receptor. BBB receptors are expressed on BBB endothelia, as well as other cell and tissue types. In some embodiments, the BBB receptor is a transferrin receptor (TfR).

In some embodiments a fusion protein described herein specifically binds to TfR. In some embodiments a fusion protein described herein specifically binds to TfR with an affinity of from about 50 nM to about 500 nM. In some embodiments, the protein binds (e.g., specifically binds) to a TfR with an affinity of about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 nM. In some embodiments, the protein binds to a TfR with an affinity of from about 100 to about 500 nM. In some embodiments, the protein binds to a TfR with an affinity of from about 100 nM to about 300 nM, or from about 150 nM to about 250 nM, or from about 200 nM to about 250 nM. In some embodiments, the protein binds to a TfR with an affinity of about 230 nM. In some embodiments, the protein binds to a TfR with an affinity of from about 150 to about 400 nM, or from about 200 to about 400 nM, or from about 250 nM to about 350 nM, or from about 300 to about 350 nM.

In some embodiments, a modified Fc polypeptide that specifically binds to TfR comprises substitutions in a CH3 domain. In some embodiments, a modified Fc polypeptide comprises a human Ig CH3 domain, such as an IgG CH3 domain, that is modified for TfR-binding activity. The CH3 domain can be of any IgG subtype, i.e., from IgG1, IgG2, IgG3, or IgG4. In the context of IgG antibodies, a CH3 domain refers to the segment of amino acids from about position 341 to about position 447 as numbered according to the EU numbering scheme.

In some embodiments, a modified Fc polypeptide that specifically binds to TfR binds to the apical domain of TfR and may bind to TfR without blocking or otherwise inhibiting binding of transferrin to TfR. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 15%, 10%, 9%, 8%, 7%, 6%, 5%, 4%, 3%, 2%, or 1%).

In some embodiments, a modified (e.g., BBB receptor-binding) Fc polypeptide present in a fusion protein described herein comprises substitutions at amino acid positions 384, 386, 387, 388, 389, 413, 415, 416, and 421, according to the EU numbering scheme.

In some embodiments, a modified Fc polypeptide that specifically binds to TfR comprises Ala at position 389, according to EU numbering. In some embodiments, a modified Fc polypeptide that specifically binds to TfR comprises at the following positions, according to EU numbering: Glu at position 380; Ala at position 389; and Asn at position 390. In some embodiments, a modified Fc polypeptide that specifically binds to TfR comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421.

In additional embodiments, the modified Fc polypeptide further comprises one, two, or three substitutions at positions comprising 414, 424, and 426, according to the EU numbering scheme. In some embodiments, position 414 is Lys, Arg, Gly, or Pro; position 424 is Ser, Thr, Glu, or Lys; and/or position 426 is Ser, Trp, or Gly.

In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to amino acids 111-217 of SEQ ID NO:32; and comprises the amino acids at EU index positions 380, 384-390 and/or 413-421 of SEQ ID NO:32. In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to amino acids 111-216 of SEQ ID NO: 33; and comprises the amino acids at EU index positions 380, 384-390 and/or 413-421 of SEQ ID NO:32 or 33. In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to SEQ ID NO:32 or 33; and comprises the amino acids at EU index positions 380, 384-390 and/or 413-421 of SEQ ID NO:32 or 33.

In some embodiments, the modified Fc polypeptide has at least 75% identity, at least 80% identity, at least 85% identity, at least 90% identity, at least 95% identity, at least 96% identity, at least 97% identity, at least 98% identity, or at least 99% identity to SEQ ID NO:32 or 33, and has Ala at position 389, according to EU numbering. In some embodiments, the modified Fc polypeptide has at least 75% identity, at least 80% identity, at least 85% identity, at least 90% identity, at least 95% identity, at least 96% identity, at least 97% identity, at least 98% identity, or at least 99% identity to SEQ ID NO:32 or 33 and comprises at the following positions, according to EU numbering: Glu at position 380; Ala at position 389; and Asn at position 390. In some embodiments, the modified Fc polypeptide has at least 75% identity, at least 80% identity, at least 85% identity, at least 90% identity, at least 95% identity, at least 96% identity, at least 97% identity, at least 98% identity, or at least 99% identity to SEQ ID NO:32 or 33 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421.

In some embodiments, the modified Fc polypeptide comprises the amino acid sequence of SEQ ID NO:32 or 33.

### Additional Fc Polypeptide Mutations

In some aspects, a fusion protein described herein comprises two Fc polypeptides, wherein one or both Fc polypeptides each comprise independently selected modifications (e.g., a modification described herein). Non-limiting examples of other mutations that can be introduced into one or both Fc polypeptides include, e.g., mutations to increase serum stability or serum half-life, to modulate effector function, to influence glycosylation, to reduce immunogenicity in humans, and/or to provide for knob and hole heterodimerization of the Fc polypeptides.

In some embodiments, the Fc polypeptides present in the fusion protein independently have an amino acid sequence identity of at least about 75%, 80%, 85%, 90%, 95%, 96%, 97%, 98%, or 99% to a corresponding wild-type Fc polypeptide (e.g., a human IgG1, IgG2, IgG3, or IgG4 Fc polypeptide).

In some embodiments, the Fc polypeptides present in the fusion protein include knob and hole mutations to promote heterodimer formation and hinder homodimer formation. Generally, the modifications introduce a protuberance (“knob”) at the interface of a first polypeptide and a corresponding cavity (“hole”) in the interface of a second polypeptide, such that the protuberance can be positioned in the cavity so as to promote heterodimer formation and thus hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). In some embodiments, such additional mutations are at a position in the Fc polypeptide that does not have a negative effect on binding of the polypeptide to a BBB receptor, e.g., TfR.

In one illustrative embodiment of a knob and hole approach for dimerization, position 366 (numbered according to the EU numbering scheme) of one of the Fc polypeptides present in the fusion protein comprises a tryptophan in place of a native threonine. The other Fc polypeptide in the dimer has a valine at position 407 (numbered according to the EU numbering scheme) in place of the native tyrosine. The other Fc polypeptide may further comprise a substitution in which the native threonine at position 366 (numbered according to the EU numbering scheme) is substituted with a serine and a native leucine at position 368 (numbered according to the EU numbering scheme) is substituted with an alanine. Thus, one of the Fc polypeptides of a fusion protein described herein has the T366W knob mutation and the other Fc polypeptide has the Y407V mutation, which is typically accompanied by the T366S and L368A hole mutations. In certain embodiments, the first Fc polypeptide contains the T366S, L368A, and Y407V substitutions and the second Fc polypeptide contains the T366W substitution. In certain other embodiments, the first Fc polypeptide contains the T366W substitution and the second Fc polypeptide contains the T366S, L368A, and Y407V substitutions.

In some embodiments, modifications to enhance serum half-life may be introduced. For example, in some embodiments, one or both Fc polypeptides present in a fusion protein described herein may comprise a tyrosine at position 252, a threonine at position 254, and a glutamic acid at position 256, as numbered according to the EU numbering scheme. Thus, one or both Fc polypeptides may have M252Y, S254T, and T256E substitutions. Alternatively, one or both Fc polypeptides may have M428L and N434S substitutions, as numbered according to the EU numbering scheme. Alternatively, one or both Fc polypeptides may have an N434S or N434A substitution.

In some embodiments, one or both Fc polypeptides present in a fusion protein described herein may comprise modifications that reduce effector function, i.e., having a reduced ability to induce certain biological functions upon binding to an Fc receptor expressed on an effector cell that mediates the effector function. Examples of antibody effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, one or both Fc polypeptides present in a fusion protein described herein may also be engineered to contain other modifications for heterodimerization, e.g., electrostatic engineering of contact residues within a CH3-CH3 interface that are naturally charged or hydrophobic patch modifications.

In some embodiments, one or both Fc polypeptides present in a fusion protein described herein may include additional modifications that modulate effector function.

In some embodiments, one or both Fc polypeptides present in a fusion protein described herein may comprise modifications that reduce or eliminate effector function. Illustrative Fc polypeptide mutations that reduce effector function include, but are not limited to, substitutions in a CH2 domain, e.g., at positions 234 and 235, according to the EU numbering scheme. For example, in some embodiments, one or both Fc polypeptides can comprise alanine residues at positions 234 and 235. Thus, one or both Fc polypeptides may have L234A and L235A (LALA) substitutions.

Additional Fc polypeptide mutations that modulate an effector function include, but are not limited to, the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII. Additional illustrative substitutions include S228P, E233P, L235E, N297A, N297D, and P331S, according to the EU numbering scheme. Multiple substitutions may also be present, e.g., L234A and L235A of a human IgG1 Fc region; L234A, L235A, and P329G of a human IgG1 Fc region; L234A, L235A, and P329S of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, according to the EU numbering scheme. In some embodiments, one or both Fc polypeptides may have one or more amino acid substitutions that modulate ADCC, e.g., substitutions at positions 298, 333, and/or 334, according to the EU numbering scheme.

In some embodiments, the C-terminal Lys residue is removed in an Fc polypeptide described herein (i.e., the Lys residue at position 447, according to the EU numbering scheme).

### Illustrative Fc Polypeptides Comprising Additional Mutations

As described herein, and by way of non-limiting example, one or both Fc polypeptides present in a fusion protein described herein may comprise additional mutations, including a knob mutation (e.g., T366W as numbered according to the EU numbering scheme), hole mutations (e.g., T366S, L368A, and Y407V as numbered according to the EU numbering scheme), mutations that modulate effector function (e.g., L234A, L235A, and/or P329G or P329S (e.g., L234A and L235A; L234A, L235A, and P329G; or L234A, L235A, and P329S)) as numbered according to the EU numbering scheme), and/or mutations that increase serum stability or serum half-life (e.g., (i) M252Y, S254T, and T256E as numbered with reference to EU numbering, or (ii) N434S with or without M428L as numbered according to the EU numbering scheme). By way of illustration, SEQ ID NOS:12-19, 24-31, 34-41 and 48-57 provide non-limiting examples of modified Fc polypeptides comprising one or more of these additional mutations.

In some embodiments, an Fc polypeptide may have a knob mutation (e.g., T366W as numbered according to the EU numbering scheme) and at least 85% identity, at least 90% identity, at least 95% identity, at least 96% identity, at least 97% identity, at least 98% identity, or at least 99% identity to the sequence of any one of SEQ ID NOS:1, 2, 32 and 33. In some embodiments, an Fc polypeptide having the sequence of any one of SEQ ID NOS: 1, 2, 32, and 33 may be modified to have a knob mutation.

In some embodiments, a modified Fc polypeptide comprises a knob mutation (e.g., T366W as numbered with reference to EU numbering) and has at least 85% identity, at least 90% identity, at least 95% identity, at least 96% identity, at least 97% identity, at least 98% identity, or at least 99% identity to the sequence of any one of SEQ ID NOS: 24 and 25. In some embodiments, the modified Fc polypeptide comprises the sequence of any one of SEQ ID NOS: 24 and 25.

In some embodiments, a modified Fc polypeptide comprises a knob mutation (e.g., T366W as numbered with reference to EU numbering) and has at least 85% identity, at least 90% identity, at least 95% identity, at least 96% identity, at least 97% identity, at least 98% identity, or at least 99% identity to the sequence of any one of SEQ ID NOS: 34 and 35, and comprises Ala at position 389, according to EU numbering. In some embodiments, a modified Fc polypeptide has at least 85% identity, at least 90% identity, at least 95% identity, at least 96% identity, at least 97% identity, at least 98% identity, or at least 99% identity to SEQ ID NO:34 or 35 and comprises at the following positions, according to EU numbering: Glu at position 380; Ala at position 389; and Asn at position 390. In some embodiments, a modified Fc polypeptide has at least 85% identity, at least 90% identity, at least 95% identity, at least 96% identity, at least 97% identity, at least 98% identity, or at least 99% identity to SEQ ID NO:34 or 35 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some embodiments, the modified Fc polypeptide comprises the sequence of any one of SEQ ID NOS: 34 and 35.

In some embodiments, an Fc polypeptide may have a knob mutation (e.g., T366W as numbered according to the EU numbering scheme), mutations that modulate effector function (e.g., L234A, L235A, and/or P329G or P329S (e.g., L234A and L235A; L234A, L235A, and P329G; or L234A, L235A, and P329S)) as numbered according to the EU numbering scheme), and at least 85% identity, at least 90% identity, at least 95% identity, at least 96% identity, at least 97% identity, at least 98% identity, or at least 99% identity to the sequence of any one of SEQ ID NOS: 1, 2, 32, and 33. In some embodiments, an Fc polypeptide having the sequence of any one of SEQ ID NOS: 1, 2, 32, and 33 may be modified to have a knob mutation and mutations that modulate effector function.

In some embodiments, a modified Fc polypeptide comprises a knob mutation (e.g., T366W as numbered with reference to EU numbering) and mutations that modulate effector function (e.g., L234A, L235A, and/or P329G or P329S (e.g., L234A and L235A; L234A, L235A, and P329G; or L234A, L235A, and P329S)) as numbered with reference to EU numbering), and has at least 85% identity, at least 90% identity, at least 95% identity, at least 96% identity, at least 97% identity, at least 98% identity, or at least 99% identity to the sequence of any one of SEQ ID NOS:26 and 27. In some embodiments, the modified Fc polypeptide comprises the sequence of any one of SEQ ID NOS: 26 and 27.

In some embodiments, a modified Fc polypeptide comprises a knob mutation (e.g., T366W as numbered with reference to EU numbering) and mutations that modulate effector function (e.g., L234A, L235A, and/or P329G or P329S (e.g., L234A and L235A; L234A, L235A, and P329G; or L234A, L235A, and P329S) as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 36-41 and 54-57, and comprises Ala at position 389, according to EU numbering. In some embodiments, a modified Fc polypeptide has at least 85% identity, at least 90% identity, at least 95% identity, at least 96% identity, at least 97% identity, at least 98% identity, or at least 99% identity to the sequence of any one of SEQ ID NOS: 36-41 and 54-57 and comprises at the following positions, according to EU numbering: Glu at position 380; Ala at position 389; and Asn at position 390. In some embodiments, a modified Fc polypeptide has at least 85% identity, at least 90% identity, at least 95% identity, at least 96% identity, at least 97% identity, at least 98% identity, or at least 99% identity to the sequence of any one of SEQ ID NOS: 36-41 and 54-57 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some embodiments, the modified Fc polypeptide comprises the sequence of any one of SEQ ID NOS: 36-41 and 54-57.

In some embodiments, an Fc polypeptide may have hole mutations (e.g., T366S, L368A, and Y407V as numbered according to the EU numbering scheme) and at least 85% identity, at least 90% identity, at least 95% identity, at least 96% identity, at least 97% identity, at least 98% identity, or at least 99% identity to the sequence of any one of SEQ ID NOS: 1, 2, 32, and 33. In some embodiments, an Fc polypeptide having the sequence of any one of SEQ ID NOS: 1, 2, 32, and 33 may be modified to have hole mutations.

In some embodiments, a modified Fc polypeptide comprises hole mutations (e.g., T366S, L368A, and Y407V as numbered with reference to EU numbering) and has at least 85% identity, at least 90% identity, at least 95% identity, at least 96% identity, at least 97% identity, at least 98% identity, or at least 99% identity to the sequence of any one of SEQ ID NOS: 12 and 13. In some embodiments, the modified Fc polypeptide comprises the sequence of any one of SEQ ID NOS: 12 and 13.

In some embodiments, a modified Fc polypeptide comprises hole mutations (e.g., T366S, L368A, and Y407V as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 48 and 49, and comprises Ala at position 389, according to EU numbering. In some embodiments, a modified Fc polypeptide has at least 85% identity, at least 90% identity, at least 95% identity, at least 96% identity, at least 97% identity, at least 98% identity, or at least 99% identity to the sequence of any one of SEQ ID NOS: 48 and 49 and comprises at the following positions, according to EU numbering: Glu at position 380; Ala at position 389; and Asn at position 390. In some embodiments, a modified Fc polypeptide has at least 85% identity, at least 90% identity, at least 95% identity, at least 96% identity, at least 97% identity, at least 98% identity, or at least 99% identity to the sequence of any one of SEQ ID NOS: 48 and 49 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some embodiments, the modified Fc polypeptide comprises the sequence of any one of SEQ ID NOS: 48 and 49.

In some embodiments, an Fc polypeptide may have hole mutations (e.g., T366S, L368A, and Y407V as numbered according to the EU numbering scheme), mutations that modulate effector function (e.g., L234A, L235A, and/or P329G or P329S (e.g., L234A and L235A; L234A, L235A, and P329G; or L234A, L235A, and P329S)) as numbered according to the EU numbering scheme), and at least 85% identity, at least 90% identity, at least 95% identity, at least 96% identity, at least 97% identity, at least 98% identity, or at least 99% identity to the sequence of any one of SEQ ID NOS: 1, 2, 32 and 33. In some embodiments, an Fc polypeptide having the sequence of any one of SEQ ID NOS: 1, 2, 32, and 33 may be modified to have hole mutations and mutations that modulate effector function.

In some embodiments, a modified Fc polypeptide comprises hole mutations (e.g., T366S, L368A, and Y407V as numbered with reference to EU numbering) and mutations that modulate effector function (e.g., L234A, L235A, and/or P329G or P329S (e.g., L234A and L235A; L234A, L235A, and P329G; or L234A, L235A, and P329S)) as numbered with reference to EU numbering), and has at least 85% identity, at least 90% identity, at least 95% identity, at least 96% identity, at least 97% identity, at least 98% identity, or at least 99% identity to the sequence of any one of SEQ ID NOS: 14-19 and 28-31. In some embodiments, the modified Fc polypeptide comprises the sequence of any one of SEQ ID NOS: 14-19 and 28-31.

In some embodiments, a modified Fc polypeptide comprises hole mutations (e.g., T366S, L368A, and Y407V as numbered with reference to EU numbering) and mutations that modulate effector function (e.g., L234A, L235A, and/or P329G or P329S (e.g., L234A and L235A; L234A, L235A, and P329G; or L234A, L235A, and P329S)) as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 50-53, and comprises Ala at position 389, according to EU numbering. In some embodiments, a modified Fc polypeptide has at least 85% identity, at least 90% identity, at least 95% identity, at least 96% identity, at least 97% identity, at least 98% identity, or at least 99% identity to the sequence of any one of SEQ ID NOS: 50-53 and comprises at the following positions, according to EU numbering: Glu at position 380; Ala at position 389; and Asn at position 390. In some embodiments, a modified Fc polypeptide has at least 85% identity, at least 90% identity, at least 95% identity, at least 96% identity, at least 97% identity, at least 98% identity, or at least 99% identity to the sequence of any one of SEQ ID NOS: 50-53 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some embodiments, the modified Fc polypeptide comprises the sequence of any one of SEQ ID NOS: 50-53.

### FcRn Binding Sites

In certain aspects, modified (e.g., BBB receptor-binding) Fc polypeptides, or Fc polypeptides present in a fusion protein described herein that do not specifically bind to a BBB receptor, can comprise an FcRn binding site. In some embodiments, the FcRn binding site is within the Fc polypeptide or a fragment thereof.

In some embodiments, the FcRn binding site comprises a native FcRn binding site. In some embodiments, the FcRn binding site does not comprise amino acid changes relative to the amino acid sequence of a native FcRn binding site. In some embodiments, the native FcRn binding site is an IgG binding site, e.g., a human IgG binding site. In some embodiments, the FcRn binding site comprises a modification that alters FcRn binding.

In some embodiments, an FcRn binding site has one or more amino acid residues that are mutated, e.g., substituted, wherein the mutation(s) increase serum half-life or do not substantially reduce serum half-life (i.e., reduce serum half-life by no more than 25% compared to a counterpart modified Fc polypeptide having the wild-type residues at the mutated positions when assayed under the same conditions). In some embodiments, an FcRn binding site has one or more amino acid residues that are substituted at positions 250-256, 307, 380, 428, and 433-436, according to the EU numbering scheme.

In some embodiments, one or more residues at or near an FcRn binding site are mutated, relative to a native human IgG sequence, to extend serum half-life of the modified polypeptide. In some embodiments, mutations are introduced into one, two, or three of positions 252, 254, and 256. In some embodiments, the mutations are M252Y, S254T, and T256E. In some embodiments, a modified Fc polypeptide further comprises the mutations M252Y, S254T, and T256E. In some embodiments, a modified Fc polypeptide comprises a substitution at one, two, or all three of positions T307, E380, and N434, according to the EU numbering scheme. In some embodiments, the mutations are T307Q and N434A. In some embodiments, a modified Fc polypeptide comprises mutations T307A, E380A, and N434A. In some embodiments, a modified Fc polypeptide comprises substitutions at positions T250 and M428, according to the EU numbering scheme. In some embodiments, the modified Fc polypeptide comprises mutations T250Q and/or M428L. In some embodiments, a modified Fc polypeptide comprises substitutions at positions M428 and N434, according to the EU numbering scheme. In some embodiments, the modified Fc polypeptide comprises mutations M428L and N434S. In some embodiments, a modified Fc polypeptide comprises an N434S or N434A mutation.

### SGSH Enzymes Linked to Fc Polypeptides

In some embodiments, a fusion protein described herein comprises two Fc polypeptides as described herein and one or both of the Fc polypeptides may further comprise a partial or full hinge region. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:5) or a portion thereof (e.g., DKTHTCPPCP; SEQ ID NO:6). In some embodiments, the hinge region is at the N-terminal region of the Fc polypeptide.

In certain embodiments, the N-terminus of the first Fc polypeptide is linked to the first SGSH amino acid sequence, SGSH variant amino acid sequence, or a catalytically active fragment thereof. In certain embodiments, the C-terminus of the first Fc polypeptide is linked to the first SGSH amino acid sequence, SGSH variant amino acid sequence, or a catalytically active fragment thereof. In certain embodiments, the N-terminus of the second Fc polypeptide is linked to the second SGSH amino acid sequence, SGSH variant amino acid sequence, or a catalytically active fragment thereof. In certain embodiments, the C-terminus of the second Fc polypeptide is linked to the second SGSH amino acid sequence, SGSH variant amino acid sequence, or a catalytically active fragment thereof.

In certain embodiments, the N-terminus of the first Fc polypeptide is linked to the first SGSH amino acid sequence, SGSH variant amino acid sequence, or a catalytically active fragment thereof, and the N-terminus of the second Fc polypeptide is linked to the second SGSH amino acid sequence, SGSH variant amino acid sequence, or a catalytically active fragment thereof.

In certain embodiments, the C-terminus of the first Fc polypeptide is linked to the first SGSH amino acid sequence, SGSH variant amino acid sequence, or a catalytically active fragment thereof, and the C-terminus of the second Fc polypeptide is linked to the second SGSH amino acid sequence, SGSH variant amino acid sequence, or a catalytically active fragment thereof.

In some embodiments, an Fc polypeptide is joined to the SGSH enzyme by a linker, e.g., a peptide linker. In some embodiments, the Fc polypeptide is joined to the SGSH enzyme by a peptide bond or by a peptide linker, e.g., is a fusion polypeptide. The peptide linker may be configured such that it allows for the rotation of the SGSH enzyme relative to the Fc polypeptide to which it is joined; and/or is resistant to digestion by proteases. Peptide linkers may contain natural amino acids, unnatural amino acids, or a combination thereof. In some embodiments, the peptide linker may be a flexible linker, e.g., containing amino acids such as Gly, Asn, Ser, Thr, Ala, and the like (e.g., a glycine-rich linker). Such linkers are designed using known parameters and may be of any length and contain any number of repeat units of any length (e.g., repeat units of Gly and Ser residues). For example, the linker may have repeats, such as two, three, four, five, or more Gly₄-Ser (SEQ ID NO:8) repeats or a single Gly₄-Ser (SEQ ID NO:8). In other aspects, the linker may be Gly-Ser. In some embodiments, the peptide linker may include a protease cleavage site, e.g., that is cleavable by an enzyme present in the central nervous system.

In some embodiments, the SGSH enzyme is joined to the N-terminus of the Fc polypeptide, e.g., by a Gly-Ser linker, a Gly₄-Ser linker (SEQ ID NO:8) or a (Gly₄-Ser)₂ linker (SEQ ID NO:9). In some embodiments, the Fc polypeptide may comprise a hinge sequence or partial hinge sequence at the N-terminus that is joined to the linker or that is directly joined to the SGSH enzyme.

In some embodiments, the SGSH enzyme is joined to the C-terminus of the Fc polypeptide, e.g., by a Gly-Ser linker, a Gly₄-Ser linker (SEQ ID NO:8) or a (Gly₄-Ser)₂ linker (SEQ ID NO:9). In some embodiments, the C-terminus of the Fc polypeptide is directly joined to the SGSH enzyme.

In some embodiments, the SGSH enzyme is joined to the Fc polypeptide by a chemical cross-linking agent. Such conjugates can be generated using well-known chemical cross-linking reagents and protocols. For example, there are a large number of chemical cross-linking agents that are known to those skilled in the art and useful for cross-linking the polypeptide with an agent of interest. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including N-hydroxysuccinimide (NHS) or its water soluble analog N-hydroxysulfosuccinimide (sulfo-NHS), succinimidyl 4-(N-maleimidomethyl)cyclohexane-1-carboxylate (SMCC), m-maleimidobenzoyl-N-hydroxysuccinimide ester (MBS); N-succinimidyl (4-iodoacetyl) aminobenzoate (SIAB), succinimidyl 4-(p-maleimidophenyl)butyrate (SMPB), 1-ethyl-3-(3-dimethylaminopropyl)carbodiimide hydrochloride (EDC); 4-succinimidyloxycarbonyl-a-methyl-a-(2-pyridyldithio)-toluene (SMPT), N-succinimidyl 3-(2-pyridyldithio)propionate (SPDP), and succinimidyl 6-[3-(2-pyridyldithio)propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exist a number of other cross-linking agents including homobifunctional and photoreactive cross-linkers. Disuccinimidyl subcrate (DSS), bismaleimidohexane (BMH) and dimethylpimelimidate. 2HCl (DMP) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers.

### Illustrative Protein Molecules Comprising SGSH Enzyme-Fc Fusion Polypeptides

In some aspects, a fusion protein described herein comprises a first Fc polypeptide that is linked to a first SGSH enzyme, SGSH enzyme variant, or a catalytically active fragment thereof, and a second Fc polypeptide that is linked to a second SGSH enzyme, SGSH enzyme variant, or a catalytically active fragment thereof, wherein the second Fc polypeptide comprises Ala at position 389, according to EU numbering; and wherein the second Fc polypeptide forms an Fc dimer with the first Fc polypeptide. In some embodiments, the second Fc polypeptide comprises at the following positions, according to EU numbering: Glu at position 380; Ala at position 389; and Asn at position 390. In some embodiments, the second Fc polypeptide comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In certain embodiments, the second Fc polypeptide specifically binds to TfR. In some embodiments, the first Fc polypeptide and/or the second Fc polypeptide does not include an immunoglobulin heavy and/or light chain variable region sequence or an antigen-binding portion thereof. In some embodiments, the first Fc polypeptide is a modified Fc polypeptide. In certain embodiments, the second Fc polypeptide (i.e., a modified Fc polypeptide) comprises one or more additional modifications. In some embodiments, a modified Fc polypeptide as described herein contains one or more modifications that promote its heterodimerization to the other Fc polypeptide. In some embodiments, a modified Fc polypeptide as described herein contains one or more modifications that reduce effector function. In some embodiments, a modified Fc polypeptide as described herein contains one or more modifications that extend serum half-life.

In some embodiments, a fusion protein described herein comprises a first polypeptide chain that comprises a first Fc polypeptide comprising T366S, L368A, and Y407V (hole) substitutions; and a second polypeptide chain that comprises a second Fc polypeptide that binds to TfR and comprises a T366W (knob) substitution. In some embodiments, the first Fc polypeptide and/or the second Fc polypeptide further comprises L234A and L235A (LALA) substitutions. In some embodiments, the first Fc polypeptide and/or the second Fc polypeptide further comprises L234A, L235A, and P329G (LALAPG) substitutions or comprises L234A, L235A, and P329S (LALAPS) substitutions. In some embodiments, the first Fc polypeptide and/or the second Fc polypeptide further comprises M252Y, S254T, and T256E (YTE) substitutions. In some embodiments, the first Fc polypeptide and/or the second Fc polypeptide further comprises: 1) L234A and L235A (LALA) substitutions; L234A, L235A, and P329G (LALAPG) substitutions; or L234A, L235A, and P329S (LALAPS) substitutions; and 2) M252Y, S254T, and T256E (YTE) substitutions. In some embodiments, the first Fc polypeptide and/or the second Fc polypeptide comprises human IgG1 wild-type residues at positions 234, 235, 252, 254, 256, and 366.

In some embodiments, the second Fc polypeptide comprises the knob; LALA/LALAPG/LALAPS, and/or YTE mutations, has at least 85% identity, at least 90% identity, at least 95% identity, at least 96% identity, at least 97% identity, at least 98% identity, or at least 99% identity to any one of SEQ ID NOS:34-41, and comprises Ala at position 389, according to EU numbering. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 34-41 and comprises at the following positions, according to EU numbering: Glu at position 380; Ala at position 389; and Asn at position 390. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 34-41 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421; or comprises the sequence of any one of SEQ ID NOS: 34-41. In some embodiments, the first Fc polypeptide comprises the hole, LALA/LALAPG/LALAPS, and/or YTE mutations, and has at least 85% identity, at least 90% identity, at least 95% identity, at least 96% identity, at least 97% identity, at least 98% identity, or at least 99% identity to any one of SEQ ID NOS:12-19; or comprises the sequence of any one of SEQ ID NOS:12-19. In some embodiments, the second Fc polypeptide comprises any one of SEQ ID NOS:34-41, and the first Fc polypeptide comprises any one of SEQ ID NOS:12-19. In some embodiments, the N-terminus of the first Fc polypeptide and/or the second Fc polypeptide includes a portion of an IgG1 hinge region (e.g., DKTHTCPPCP; SEQ ID NO:6). In some embodiments, the second Fc polypeptide has at least 85%, at least 90%, at least 95% identity, at least 96% identity, at least 97% identity, at least 98% identity, or at least 99% identity to any one of SEQ ID NOS: 54-57, and comprises Ala at position 389, according to EU numbering. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 54-57 and comprises at the following positions, according to EU numbering: Glu at position 380; Ala at position 389; and Asn at position 390. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 54-57 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421, or comprises the sequence of any one of SEQ ID NOS:54-57. In some embodiments, the first Fc polypeptide has at least 85%, at least 90%, at least 95% identity, at least 96% identity, at least 97% identity, at least 98% identity, or at least 99% identity to any one of SEQ ID NOS: 28-31, or comprises the sequence of any one of SEQ ID NOS:28-31.

In some embodiments, a fusion protein described herein comprises a first polypeptide chain that comprises a first Fc polypeptide comprising a T366W (knob) substitution; and a second polypeptide chain that comprises a second Fc polypeptide that binds to TfR and comprises T366S, L368A, and Y407V (hole) substitutions. In some embodiments, the first Fc polypeptide and/or the second Fc polypeptide further comprises L234A and L235A (LALA) substitutions. In some embodiments, the first Fc polypeptide and/or the second Fc polypeptide further comprises L234A, L235A, and P329G (LALAPG) substitutions or comprises L234A, L235A, and P329S (LALAPS) substitutions. In some embodiments, the first Fc polypeptide and/or the second Fc polypeptide further comprises M252Y, S254T, and T256E (YTE) substitutions. In some embodiments, the first Fc polypeptide and/or the second Fc polypeptide further comprises: 1) L234A and L235A (LALA) substitutions; L234A, L235A, and P329G (LALAPG) substitutions; or L234A, L235A, and P329S (LALAPS) substitutions; and 2) M252Y, S254T, and T256E (YTE) substitutions. In some embodiments, the first Fc polypeptide and/or the second Fc polypeptide comprises human IgG1 wild-type residues at positions 234, 235, 252, 254, 256, and 366.

In some embodiments, the second Fc polypeptide comprises the hole, LALA/LALAPG/LALAPS, and/or YTE mutations, has at least 85% identity, at least 90% identity, at least 95% identity, at least 96% identity, at least 97% identity, at least 98% identity, or at least 99% identity to any one of SEQ ID NOS:48-53, and comprises Ala at position 389, according to EU numbering. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 48-53 and comprises at the following positions, according to EU numbering: Glu at position 380; Ala at position 389; and Asn at position 390. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 48-53 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421; or comprises the sequence of any one of SEQ ID NOS:48-53. In some embodiments, the first Fc polypeptide comprises the knob, LALA/LALAPG/LALAPS, and/or YTE mutations and has at least 85% identity, at least 90% identity, at least 95% identity, at least 96% identity, at least 97% identity, at least 98% identity, or at least 99% identity to any one of SEQ ID NOS:24-27; or comprises the sequence of any one of SEQ ID NOS: 24-27. In some embodiments, the second Fc polypeptide comprises any one of SEQ ID NOS: 48-53, and the first Fc polypeptide comprises any one of SEQ ID NOS:24-27. In some embodiments, the N-terminus of a modified Fc polypeptide and/or a Fc polypeptide includes a portion of an IgG1 hinge region (e.g., DKTHTCPPCP; SEQ ID NO:6).

In some embodiments, a first SGSH enzyme, present in a fusion protein described herein is linked to a first polypeptide chain that comprises a first Fc polypeptide having at least 85%, at least 90%, at least 95% identity, at least 96% identity, at least 97% identity, at least 98% identity, or at least 99% identity to any one of SEQ ID NOS: 12-19, or comprises the sequence of any one of SEQ ID NOS: 12-19 (e.g., as a fusion polypeptide). In some embodiments, the first SGSH enzyme is linked to the first Fc polypeptide by a linker, such as a flexible linker, and/or a hinge region or portion thereof (e.g., DKTHTCPPCP; SEQ ID NO:6). In some embodiments, the N-terminus of the first Fc polypeptide includes a portion of an IgG1 hinge region (e.g., DKTHTCPPCP; SEQ ID NO:6). In some embodiments, the first Fc polypeptide has at least 85%, at least 90%, at least 95% identity, at least 96% identity, at least 97% identity, at least 98% identity, or at least 99% identity to any one of SEQ ID NOS:28-31, or comprises the sequence of any one of SEQ ID NOS:28-31. In some embodiments, the first SGSH enzyme comprises an SGSH sequence having at least 85%, at least 90%, at least 95% identity, at least 96% identity, at least 97% identity, at least 98% identity, or at least 99% identity to any one of SEQ ID NO:58-60, or comprises the sequence of any one of SEQ ID NO:58-60. In some embodiments, the first SGSH sequence linked to the Fc polypeptide has at least 85%, at least 90%, at least 95% identity, at least 96% identity, at least 97% identity, at least 98% identity, or at least 99% identity to any one of SEQ ID NOS:61-68, 73-76, 81-84 and 117-118, or comprises the sequence of any one of SEQ ID NOS:61-68, 73-76, 81-84 and 117-118. In some embodiments, the fusion protein comprises a second Fc polypeptide having at least 85%, at least 90%, at least 95% identity, at least 96% identity, at least 97% identity, at least 98% identity, or at least 99% identity to any one of SEQ ID NOS: 34-41, and comprises Ala at position 389, according to EU numbering. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 34-41 and comprises at the following positions, according to EU numbering: Glu at position 380; Ala at position 389; and Asn at position 390. In some embodiments, the second polypeptide has at least 85% identity, at least 90% identity, at least 95% identity, at least 96% identity, at least 97% identity, at least 98% identity, or at least 99% identity to the sequence of any one of SEQ ID NOS: 34-41 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421, or comprises the sequence of any one of SEQ ID NOS: 34-41. In some embodiments, a second SGSH enzyme is linked to the second Fc polypeptide by a linker, such as a flexible linker, and/or a hinge region or portion thereof (e.g., DKTHTCPPCP; SEQ ID NO:6). In some embodiments, the N-terminus of the second Fc polypeptide includes a portion of an IgG1 hinge region (e.g., DKTHTCPPCP; SEQ ID NO:6). In some embodiments, the second Fc polypeptide has at least 85%, at least 90%, at least 95% identity, at least 96% identity, at least 97% identity, at least 98% identity, or at least 99% identity to any one of SEQ ID NOS:54-57, and comprises Ala at position 389, according to EU numbering. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 54-57 and comprises at the following positions, according to EU numbering: Glu at position 380; Ala at position 389; and Asn at position 390. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 54-57 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421, or comprises the sequence of any one of SEQ ID NOS:54-57. In some embodiments, the second SGSH enzyme comprises an SGSH sequence having at least 85%, at least 90%, at least 95% identity, at least 96% identity, at least 97% identity, at least 98% identity, or at least 99% identity to any one of SEQ ID NO:58-60, or comprises the sequence of any one of SEQ ID NO:58-60. In some embodiments, the second SGSH sequence linked to the second Fc polypeptide has at least 85%, at least 90%, at least 95% identity, at least 96% identity, at least 97% identity, at least 98% identity, or at least 99% identity to any one of SEQ ID NOS: 89-96, 101-104, 109-112, and 119-120, and comprises Ala at position 389, according to EU numbering. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 89-96, 101-104, 109-112, and 119-120 and comprises at the following positions, according to EU numbering: Glu at position 380; Ala at position 389; and Asn at position 390. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 89-96, 101-104, 109-112, and 119-120 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421, or comprises the sequence of any one of SEQ ID NOS: 89-96, 101-104, 109-112, and 119-120.

In some embodiments, the fusion protein comprises a first SGSH-Fc fusion polypeptide comprising the sequence of any one of SEQ ID NOS:61-64, and a second SGSH-Fc polypeptide comprising the sequence of any one of SEQ ID NO:89-92.

In some embodiments, the fusion protein comprises a first SGSH-Fc fusion polypeptide comprising the sequence of any one of SEQ ID NOS:65-68, and a second SGSH-Fc polypeptide comprising the sequence of any one of SEQ ID NO:93-96.

In some embodiments, the fusion protein comprises a first SGSH-Fc fusion polypeptide comprising the sequence of any one of SEQ ID NOS:73-76, and a second SGSH-Fc polypeptide comprising the sequence of any one of SEQ ID NO:101-104.

In some embodiments, the fusion protein comprises a first SGSH-Fc fusion polypeptide comprising the sequence of any one of SEQ ID NOS:81-84, and a second SGSH-Fc polypeptide comprising the sequence of any one of SEQ ID NO:109-112.

In some embodiments, the fusion protein comprises a first SGSH-Fc fusion polypeptide comprising the sequence of any one of SEQ ID NOS:117-118, and a second SGSH-Fc polypeptide comprising the sequence of any one of SEQ ID NO:119-120.

In some embodiments, the fusion protein comprises a first SGSH-Fc fusion polypeptide comprising the sequence of SEQ ID NO:61 or 62, and a second SGSH-Fc polypeptide comprising the sequence of SEQ ID NO:89 or 90.

In some embodiments, the fusion protein comprises a first SGSH-Fc fusion polypeptide comprising the sequence of SEQ ID NO:65 or 66, and a second SGSH-Fc polypeptide comprising the sequence of SEQ ID NO:93 or 94.

In some embodiments, the fusion protein comprises a first SGSH-Fc fusion polypeptide comprising the sequence of SEQ ID NO:63 or 64, and a second SGSH-Fc polypeptide comprising the sequence of SEQ ID NO:91 or 92.

In some embodiments, the fusion protein comprises a first SGSH-Fc fusion polypeptide comprising the sequence of SEQ ID NO:64, and a second SGSH-Fc polypeptide comprising the sequence of SEQ ID NO:92.

In some embodiments, the fusion protein comprises a first SGSH-Fc fusion polypeptide comprising the sequence of SEQ ID NO:67 or 68, and a second SGSH-Fc polypeptide comprising the sequence of SEQ ID NO:95 or 96.

In some embodiments, the fusion protein comprises a first SGSH-Fc fusion polypeptide comprising the sequence of SEQ ID NO:68, and a second SGSH-Fc polypeptide comprising the sequence of SEQ ID NO:96.

In some embodiments, the fusion protein comprises a first SGSH-Fc fusion polypeptide comprising the sequence of SEQ ID NO:73 or 74, and a second SGSH-Fc polypeptide comprising the sequence of SEQ ID NO:101 or 102.

In some embodiments, the fusion protein comprises a first SGSH-Fc fusion polypeptide comprising the sequence of SEQ ID NO:75 or 76, and a second SGSH-Fc polypeptide comprising the sequence of SEQ ID NO:103 or 104.

In some embodiments, the fusion protein comprises a first SGSH-Fc fusion polypeptide comprising the sequence of SEQ ID NO:76, and a second SGSH-Fc polypeptide comprising the sequence of SEQ ID NO:104.

In some embodiments, the fusion protein comprises a first SGSH-Fc fusion polypeptide comprising the sequence of SEQ ID NO:81 or 82, and a second SGSH-Fc polypeptide comprising the sequence of SEQ ID NO:109 or 110.

In some embodiments, the fusion protein comprises a first SGSH-Fc fusion polypeptide comprising the sequence of SEQ ID NO:83 or 84, and a second SGSH-Fc polypeptide comprising the sequence of SEQ ID NO:111 or 112.

In some embodiments, the fusion protein comprises a first SGSH-Fc fusion polypeptide comprising the sequence of SEQ ID NO:84, and a second SGSH-Fc polypeptide comprising the sequence of SEQ ID NO:112.

In some embodiments, the fusion protein comprises a first SGSH-Fc fusion polypeptide comprising the sequence of SEQ ID NO:117, and a second SGSH-Fc polypeptide comprising the sequence of SEQ ID NO:119.

In some embodiments, the fusion protein comprises a first SGSH-Fc fusion polypeptide comprising the sequence of SEQ ID NO:118, and a second SGSH-Fc polypeptide comprising the sequence of SEQ ID NO:120.

In some embodiments, a first SGSH enzyme, present in a fusion protein described herein is linked to a first polypeptide chain that comprises a first Fc polypeptide having at least 85%, at least 90%, at least 95% identity, at least 96% identity, at least 97% identity, at least 98% identity, or at least 99% identity to any one of SEQ ID NOS: 24-27, or comprises the sequence of any one of SEQ ID NOS: 24-27 (e.g., as a fusion polypeptide). In some embodiments, the first SGSH enzyme is linked to the first Fc polypeptide by a linker, such as a flexible linker, and/or a hinge region or portion thereof (e.g., DKTHTCPPCP; SEQ ID NO:6). In some embodiments, the N-terminus of the first Fc polypeptide includes a portion of an IgG1 hinge region (e.g., DKTHTCPPCP; SEQ ID NO:6). In some embodiments, the first SGSH enzyme comprises an SGSH sequence having at least 85%, at least 90%, at least 95% identity, at least 96% identity, at least 97% identity, at least 98% identity, or at least 99% identity to any one of SEQ ID NO:58-60, or comprises the sequence of any one of SEQ ID NO:58-60. In some embodiments, the first SGSH sequence linked to the Fc polypeptide has at least 85%, at least 90%, at least 95% identity, at least 96% identity, at least 97% identity, at least 98% identity, or at least 99% identity to any one of SEQ ID NOS:69-72, 77-80, and 85-88, or comprises the sequence of any one of SEQ ID NOS: 69-72, 77-80, and 85-88. In some embodiments, the fusion protein comprises a second Fc polypeptide having at least 85%, at least 90%, at least 95% identity, at least 96% identity, at least 97% identity, at least 98% identity, or at least 99% identity to any one of SEQ ID NOS: 48-53, and comprises Ala at position 389, according to EU numbering. In some embodiments, the second polypeptide has at least 85% identity, at least 90% identity, at least 95% identity, at least 96% identity, at least 97% identity, at least 98% identity, or at least 99% identity to the sequence of any one of SEQ ID NOS: 48-53 and comprises at the following positions, according to EU numbering: Glu at position 380; Ala at position 389; and Asn at position 390. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 48-53 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421, or comprises the sequence of any one of SEQ ID NOS:48-53. In some embodiments, a second SGSH enzyme is linked to the second Fc polypeptide by a linker, such as a flexible linker, and/or a hinge region or portion thereof (e.g., DKTHTCPPCP; SEQ ID NO:6). In some embodiments, the N-terminus of the second Fc polypeptide includes a portion of an IgG1 hinge region (e.g., DKTHTCPPCP; SEQ ID NO:6). In some embodiments, the second SGSH enzyme comprises an SGSH sequence having at least 85%, at least 90%, at least 95% identity, at least 96% identity, at least 97% identity, at least 98% identity, or at least 99% identity to any one of SEQ ID NO:58-60, or comprises the sequence of any one of SEQ ID NO:58-60. In some embodiments, the second SGSH sequence linked to the second Fc polypeptide has at least 85%, at least 90%, at least 95% identity, at least 96% identity, at least 97% identity, at least 98% identity, or at least 99% identity to any one of SEQ ID NOS: 97-100, 105-108, and 113-116, and comprises Ala at position 389, according to EU numbering. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 97-100, 105-108, and 113-116 and comprises at the following positions, according to EU numbering: Glu at position 380; Ala at position 389; and Asn at position 390. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 97-100, 105-108, and 113-116 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421, or comprises the sequence of any one of SEQ ID NOS: 97-100, 105-108, and 113-116.

In some embodiments, the fusion protein comprises a first SGSH-Fc fusion polypeptide comprising the sequence of SEQ ID NO:69 or 70, and a second SGSH-Fc polypeptide comprising the sequence of SEQ ID NO:97 or 98.

In some embodiments, the fusion protein comprises a first SGSH-Fc fusion polypeptide comprising the sequence of SEQ ID NO:71 or 72, and a second SGSH-Fc polypeptide comprising the sequence of SEQ ID NO:99 or 100.

In some embodiments, the fusion protein comprises a first SGSH-Fc fusion polypeptide comprising the sequence of SEQ ID NO:77 or 78, and a second SGSH-Fc polypeptide comprising the sequence of SEQ ID NO:105 or 106.

In some embodiments, the fusion protein comprises a first SGSH-Fc fusion polypeptide comprising the sequence of SEQ ID NO:79 or 80, and a second SGSH-Fc polypeptide comprising the sequence of SEQ ID NO:107 or 108.

In some embodiments, the fusion protein comprises a first SGSH-Fc fusion polypeptide comprising the sequence of SEQ ID NO:85 or 86, and a second SGSH-Fc polypeptide comprising the sequence of SEQ ID NO:113-114.

In some embodiments, the fusion protein comprises a first SGSH-Fc fusion polypeptide comprising the sequence of SEQ ID NO:87 or 88, and a second SGSH-Fc polypeptide comprising the sequence of SEQ ID NO:115-116.

Fusion proteins and other compositions described herein may have a range of binding affinities. For example, in some embodiments, a protein has an affinity for a transferrin receptor (TfR), ranging anywhere from 1 pM to 10 μM. In some embodiments, the affinity for TfR ranges from 1 nM to 5 pM, or from 10 nM to 1 μM. In some embodiments, the affinity for TfR ranges from about 50 mM to about 500 nM, or from about 100 nM to about 500 nM. In some embodiments, the affinity for TfR ranges from about 50 nM to about 300 nM. In some embodiments, the affinity for TfR ranges from about 100 nM to about 350 nM. In some embodiments, the affinity for TfR ranges from about 150 nM to about 400 nM. In some embodiments, the affinity for TfR ranges from about 200 nM to about 450 nM. In some embodiments, the affinity for TfR is a monovalent affinity.

### Evaluation of Protein Activity

Activity of fusion proteins described herein that comprise SGSH enzymes can be assessed using various assays, including assays that measure activity in vitro using an artificial substrate, such as those described in the Examples section. Other illustrative protocols for measuring SGSH activity in vitro are provided, e.g., in WO2019/070577.

In some embodiments, a tissue sample is evaluated. A tissue sample can be evaluated using an assay as described above, except multiple free-thaw cycles, e.g., 2, 3, 4, 5, or more, are typically included before the sonication step to ensure that microvesicles are broken open.

Samples that can be evaluated by the assays described herein include brain, liver, kidney, lung, spleen, plasma, serum, cerebrospinal fluid (CSF), and urine. In some embodiments, CSF samples from a patient receiving an enzyme-Fc fusion protein (e.g., SGSH-Fc fusion protein) described herein may be evaluated.

### Nucleic Acids, Vectors, and Host Cells

Polypeptide chains contained in the fusion proteins as described herein are typically prepared using recombinant methods. Accordingly, in some aspects, the present disclosure provides isolated nucleic acids comprising a nucleic acid sequence encoding any of the polypeptide chains comprising Fc polypeptides as described herein, and host cells into which the nucleic acids are introduced that are used to replicate the polypeptide-encoding nucleic acids and/or to express the polypeptides. In some embodiments, the host cell is eukaryotic, e.g., a human cell.

In another aspect, polynucleotides are provided that comprise a nucleotide sequence that encodes one or more of the polypeptide chains described herein. In some embodiments, the polynucleotide encodes one of the polypeptide sequences described here. In some embodiments, the polynucleotide encodes two of the polypeptide sequences described herein. The polynucleotides may be single-stranded or double-stranded. In some embodiments, the polynucleotide is DNA. In particular embodiments, the polynucleotide is cDNA. In some embodiments, the polynucleotide is RNA.

Some embodiments also provide a pair of nucleic acid sequences, wherein each nucleic acid sequence encodes a polypeptide described herein. For example, certain embodiments provide a pair of nucleic acid sequences, wherein a first nucleic acid sequence in the pair encodes a first Fc polypeptide linked to a first SGSH amino acid sequence, SGSH variant amino acid sequence, or a catalytically active fragment thereof, and a second nucleic acid sequence in the pair encodes a second Fc polypeptide linked to a second SGSH amino acid sequence, SGSH variant amino acid sequence, or a catalytically active fragment thereof.

In some embodiments, the polynucleotide is included within a nucleic acid construct or the pair of polynucleotides is included within one or more nucleic acid constructs. In some embodiments, the construct is a replicable vector. In some embodiments, the vector is selected from a plasmid, a viral vector, a phagemid, a yeast chromosomal vector, and a non-episomal mammalian vector.

In some embodiments, the polynucleotide is operably linked to one or more regulatory nucleotide sequences in an expression construct. In one series of embodiments, the nucleic acid expression constructs are adapted for use as a surface expression library. In some embodiments, the library is adapted for surface expression in yeast. In some embodiments, the library is adapted for surface expression in phage. In another series of embodiments, the nucleic acid expression constructs are adapted for expression of the polypeptide in a system that permits isolation of the polypeptide in milligram or gram quantities. In some embodiments, the system is a mammalian cell expression system. In some embodiments, the system is a yeast cell expression system.

Expression vehicles for production of a recombinant polypeptide include plasmids and other vectors. For instance, suitable vectors include plasmids of the following types: pBR322-derived plasmids, pEMBL-derived plasmids, pEX-derived plasmids, pBTac-derived plasmids, and pUC-derived plasmids for expression in prokaryotic cells, such as E. coli. The pcDNAI/amp, pcDNAI/neo, pRc/CMV, pSV2gpt, pSV2neo, pSV2-dhfr, pTk2, pRSVneo, pMSG, pSVT7, pko-neo, and pHyg-derived vectors are examples of mammalian expression vectors suitable for transfection of eukaryotic cells. Alternatively, derivatives of viruses such as the bovine papilloma virus (BPV-1), or Epstein-Barr virus (pHEBo, pREP-derived, and p205) can be used for transient expression of polypeptides in eukaryotic cells. In some embodiments, it may be desirable to express the recombinant polypeptide by the use of a baculovirus expression system. Examples of such baculovirus expression systems include pVL-derived vectors (such as pVL1392, pVL1393, and pVL941), pAcUW-derived vectors (such as pAcUW1), and pBlueBac-derived vectors. Additional expression systems include adenoviral, adeno-associated virus, and other viral expression systems.

Vectors may be transformed into any suitable host cell. In some embodiments, the host cells, e.g., bacteria or yeast cells, may be adapted for use as a surface expression library. In some cells, the vectors are expressed in host cells to express relatively large quantities of the polypeptide. Such host cells include mammalian cells, yeast cells, insect cells, and prokaryotic cells. In some embodiments, the cells are mammalian cells, such as Chinese Hamster Ovary (CHO) cell, baby hamster kidney (BHK) cell, NS0 cell, Y0 cell, HEK293 cell, COS cell, Vero cell, or HeLa cell.

A host cell transfected with an expression vector(s) encoding one or more Fc polypeptide chains as described herein can be cultured under appropriate conditions to allow expression of the one or more polypeptides to occur. The polypeptides may be secreted and isolated from a mixture of cells and medium containing the polypeptides. Alternatively, the polypeptides may be retained in the cytoplasm or in a membrane fraction and the cells harvested, lysed, and the polypeptide isolated using a desired method.

### Therapeutic Methods

A fusion protein as described herein may be used therapeutically to treat Sanfilippo syndrome A.

Accordingly, certain embodiments provide a method of decreasing the accumulation of a toxic metabolic product (e.g., a heparan sulfate-derived oligosaccharide) in a subject having Sanfilippo syndrome A, the method comprising administering a protein as described herein to the subject.

Certain embodiments provide a protein as described herein for use in decreasing the accumulation of a toxic metabolic product (e.g., a heparan sulfate-derived oligosaccharide) in a subject having Sanfilippo syndrome A.

Certain embodiments provide the use of a protein as described herein in the preparation of a medicament for decreasing the accumulation of a toxic metabolic product (e.g., a heparan sulfate-derived oligosaccharide) in a subject having Sanfilippo syndrome A.

Certain embodiments also provide a method of treating Sanfilippo syndrome A, comprising administering a protein as described herein to a subject in need thereof.

Certain embodiments provide a protein as described herein for use in treating Sanfilippo syndrome A in a subject in need thereof.

Certain embodiments provide the use of a protein as described herein in the preparation of a medicament for treating Sanfilippo syndrome A in a subject in need thereof.

In some embodiments, administration of the protein (e.g., linked to SGSH enzymes) improves (e.g., increases) Cₘₐₓ of SGSH in the brain as compared to the uptake of SGSH in the absence of being linked to a fusion protein described herein or as compared to the uptake of SGSH linked to a reference protein (e.g., a fusion protein as described herein, which does not have the modifications to the second Fc polypeptide that result in TfR binding).

In some embodiments, Cₘₐₓ of SGSH in the brain is improved (e.g., increased) by at least about 1.1-fold, 1.2-fold, 1.3-fold, 1.4-fold, 1.5-fold, 1.6-fold, 1.7-fold, 1.8-fold, 1.9-fold, 2-fold, 2.2-fold, 2.4-fold, 2.6-fold, 2.8-fold, 3-fold, 4-fold, 5-fold, 6-fold, or more, as compared to the uptake of SGSH in the absence of being linked to a fusion protein described herein or as compared to the uptake of SGSH linked to a reference protein (e.g., a fusion protein as described herein, which does not have the modifications to the second Fc polypeptide that result in TfR binding).

A fusion protein described herein is administered to a subject at a therapeutically effective amount or dose.

In various embodiments, a fusion protein described herein is administered parenterally. In some embodiments, the protein is administered intravenously.

In some parenteral embodiments, a fusion protein as described herein is administered intraperitoneally, subcutaneously, intradermally, or intramuscularly. In some embodiments, the fusion protein as described herein is administered intradermally or intramuscularly. In some embodiments, the fusion protein as described herein is administered intrathecally, such as by epidural administration, or intracerebroventricularly.

In other embodiments, a fusion protein as described herein may be administered orally, by pulmonary administration, intranasal administration, intraocular administration, or by topical administration. Pulmonary administration can also be employed, e.g., by use of an inhaler or nebulizer, and formulation with an aerosolizing agent.

### Pharmaceutical Compositions and Kits

In other aspects, pharmaceutical compositions and kits comprising a fusion protein described herein are provided.

### Pharmaceutical Compositions

Guidance for preparing formulations for use in the present disclosure can be found in any number of handbooks for pharmaceutical preparation and formulation that are known to those of skill in the art.

In some embodiments, a pharmaceutical composition comprises a fusion protein as described herein and further comprises one or more pharmaceutically acceptable carriers and/or excipients. A pharmaceutically acceptable carrier includes any solvents, dispersion media, or coatings that are physiologically compatible and that do not interfere with or otherwise inhibit the activity of the active agent.

Dosages and desired drug concentration of pharmaceutical compositions described herein may vary depending on the particular use envisioned. Exemplary dosages are described herein.

### Kits

In some embodiments, a kit for use in treating Sanfilippo syndrome A, comprising a fusion protein as described herein, is provided.

In some embodiments, the kit further comprises one or more additional therapeutic agents. For example, in some embodiments, the kit comprises a fusion protein as described herein and further comprises one or more additional therapeutic agents for use in the treatment of neurological symptoms of Sanfilippo syndrome A. In some embodiments, the kit further comprises instructional materials containing directions (i.e., protocols) for the practice of the methods described herein (e.g., instructions for using the kit for administering a fusion protein comprising an SGSH enzyme across the blood-brain barrier). While the instructional materials typically comprise written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD-ROM), and the like. Such media may include addresses to internet sites that provide such instructional materials.

### Certain Definitions

As used herein, the singular forms “a,” “an,” and “the” include plural referents unless the content clearly dictates otherwise. Thus, for example, reference to “a polypeptide” may include two or more such molecules, and the like.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the patient is a human. In some embodiments, the human is a patient in need of treatment for Sanfilippo syndrome A. In some embodiments, the patient has one or more signs or symptoms of Sanfilippo syndrome A.

The term “pharmaceutically acceptable excipient” refers to a non-active pharmaceutical ingredient that is biologically or pharmacologically compatible for use in humans or animals, such as but not limited to a buffer, carrier, or preservative.

The term “administer” refers to a method of delivering agents (e.g., a Sanfilippo syndrome A therapeutic agent, such as an ETV:SGSH therapy described herein), compounds, or compositions (e.g., pharmaceutical composition) to the desired site of biological action. These methods include, but are not limited to, oral, topical delivery, parenteral delivery, intravenous delivery, intradermal delivery, intramuscular delivery, intrathecal delivery, or intraperitoneal delivery. In one embodiment, the polypeptides described herein are administered intravenously.

As used herein, “treatment” (and grammatical variations thereof such as “treat” or “treating”) refers to clinical intervention to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis.

The phrase “effective amount” means an amount of a compound described herein that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

A “therapeutically effective amount” of a substance/molecule disclosed herein may vary according to factors such as the disease state, age, sex, and weight of the individual, and the ability of the substance/molecule, to elicit a desired response in the individual. A therapeutically effective amount encompasses an amount in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

A “sulfoglucosamine sulfohydrolase,” “N-sulfoglucosamine sulfohydrolase,” or “SGSH” as used herein refers to N-sulfoglucosamine sulfohydrolase (EC 3.10.1.1), which is an enzyme involved in the lysosomal degradation of heparan sulfate. Mutations in this gene are associated with Sanfilippo syndrome A, one type of the lysosomal storage disorder mucopolysaccaridosis III, which results from impaired degradation of heparan sulfate. The term “SGSH” as used herein as a component of a protein that comprises an Fc polypeptide is catalytically active and encompasses functional variants, including allelic and splice variants, of a wild-type SGSH or a fragment thereof. The sequence of human SGSH is available under UniProt entry P51688 and is encoded by the human SGSH gene at 17q25.3. The full-length sequence is provided as SEQ ID NO:58. A “mature” SGSH sequence as used herein refers to a form of a polypeptide chain that lacks the signal sequence of the naturally occurring full-length polypeptide chain. The amino acid sequence of a mature human SGSH polypeptide is provided as SEQ ID NO:59, which corresponds to amino acids 21-502 of the full-length human sequence. A “truncated” SGSH sequence as used herein refers to a catalytically active fragment of the naturally occurring full-length polypeptide chain. The structure of human SGSH has been well-characterized. An illustrative structure is available under PDB accession code 4MHX. Non-human primate SGSH sequences have also been described, including chimpanzee (UniProt entry K7C218). A mouse SGSH sequence is available under Uniprot entry Q9EQ08. An SGSH variant has at least 50%, at least 55%, at least 60%, at least 65%, at least 70%, at least 75%, at least 80%, at least 85%, at least 90%, or at least 95% of the activity of the corresponding wild-type SGSH or fragment thereof, e.g., when assayed under identical conditions. A catalytically active SGSH fragment has at least 50%, at least 55%, at least 60%, at least 65%, at least 70%, at least 75%, at least 80%, at least 85%, at least 90%, or at least 95% of the activity of the corresponding full-length SGSH or variant thereof, e.g., when assayed under identical conditions.

A “transferrin receptor” or “TfR” as used herein refers to transferrin receptor protein 1. The human transferrin receptor 1 polypeptide sequence is set forth in SEQ ID NO:10. Transferrin receptor protein 1 sequences from other species are also known (e.g., chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full-length transferrin receptor protein includes a short N-terminal intracellular region, a transmembrane region, and a large extracellular domain. The extracellular domain is characterized by three domains: a protease-like domain, a helical domain, and an apical domain. The apical domain sequence of human transferrin receptor 1 is set forth in SEQ ID NO:11.

A “fusion protein” or “[SGSH enzyme]-Fc fusion protein” as used herein refers to a dimeric protein comprising a first Fc polypeptide that is linked (e.g., fused) to an SGSH enzyme, an SGSH enzyme variant, or a catalytically active fragment thereof (i.e., an “[SGSH]-Fc fusion polypeptide”); and a second Fc polypeptide that forms an Fc dimer with the first Fc polypeptide. The second Fc polypeptide may also be linked (e.g., fused) to an SGSH enzyme, an SGSH enzyme variant, or a catalytically active fragment thereof. The first Fc polypeptide and/or the second Fc polypeptide may be linked to the SGSH enzyme, SGSH enzyme variant, or catalytically active fragment thereof by a peptide bond or by a polypeptide linker. The first Fc polypeptide and/or the second Fc polypeptide may be a modified Fc polypeptide that contains one or more modifications that promote its heterodimerization to the other Fc polypeptide. The first Fc polypeptide and/or the second Fc polypeptide may be a modified Fc polypeptide that contains one or more modifications that confer binding to a transferrin receptor. The first Fc polypeptide and/or the second Fc polypeptide may be a modified Fc polypeptide that contains one or more modifications that reduce effector function. In certain embodiments, the first Fc polypeptide and the second Fc polypeptide do not have effector function. The first Fc polypeptide and/or the second Fc polypeptide may be a modified Fc polypeptide that contains one or more modifications that extend serum half-life. In certain embodiments, the first Fc polypeptide and/or the second Fc polypeptide do not include an immunoglobulin heavy and/or light chain variable region sequence or an antigen-binding portion thereof. In certain embodiments, the first Fc polypeptide and the second Fc polypeptide do not include an immunoglobulin heavy and/or light chain variable region sequence or an antigen-binding portion thereof.

A “fusion polypeptide” or “[SGSH enzyme]-Fc fusion polypeptide” as used herein refers to an Fc polypeptide that is linked (e.g., fused) to an SGSH enzyme, an SGSH enzyme variant, or a catalytically active fragment thereof. The Fc polypeptide may be linked to the SGSH enzyme, SGSH enzyme variant, or catalytically active fragment thereof by a peptide bond or by a polypeptide linker. The Fc polypeptide may be a modified Fc polypeptide that contains one or more modifications that promote its heterodimerization to another Fc polypeptide. The Fc polypeptide may be a modified Fc polypeptide that contains one or more modifications that confer binding to a transferrin receptor. The Fc polypeptide may be a modified Fc polypeptide that contains one or more modifications that reduce effector function. The Fc polypeptide may be a modified Fc polypeptide that contains one or more modifications that extend serum half-life.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region. In general, an Fc polypeptide does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

The term “FcRn” refers to the neonatal Fc receptor. Binding of Fc polypeptides to FcRn reduces clearance and increases serum half-life of the Fc polypeptide. The human FcRn protein is a heterodimer that is composed of a protein of about 50 kDa in size that is similar to a major histocompatibility (MHC) class I protein and a P2-microglobulin of about 15 kDa in size.

As used herein, an “FcRn binding site” refers to the region of an Fc polypeptide that binds to FcRn. In human IgG, the FcRn binding site, as numbered using the EU index, includes T250, L251, M252, I253, S254, R255, T256, T307, E380, M428, H433, N434, H435, and Y436. These positions correspond to positions 20 to 26, 77, 150, 198, and 203 to 206 of SEQ ID NO:1.

As used herein, a “native FcRn binding site” refers to a region of an Fc polypeptide that binds to FcRn and that has the same amino acid sequence as the region of a naturally occurring Fc polypeptide that binds to FcRn.

The terms “CH3 domain” and “CH2 domain” as used herein refer to immunoglobulin constant region domain polypeptides. For purposes of this application, a CH3 domain polypeptide refers to the segment of amino acids from about position 341 to about position 447 as numbered according to EU, and a CH2 domain polypeptide refers to the segment of amino acids from about position 231 to about position 340 as numbered according to the EU numbering scheme and does not include hinge region sequences. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. An Fc region refers to the segment of amino acids from about position 231 to about position 447 as numbered according to the EU numbering scheme, but as used herein, can include at least a part of a hinge region of an antibody. An illustrative hinge region sequence is the human IgG1 hinge sequence EPKSCDKTHTCPPCP (SEQ ID NO:5).

“Naturally occurring,” “native” or “wild type” is used to describe an object that can be found in nature as distinct from being artificially produced. For example, a nucleotide sequence present in an organism (including a virus), which can be isolated from a source in nature and which has not been intentionally modified in the laboratory, is naturally occurring. Furthermore, “wild-type” refers to the normal gene, or organism found in nature without any known mutation. For example, the terms “wild-type,” “native,” and “naturally occurring” with respect to a CH3 or CH2 domain are used herein to refer to a domain that has a sequence that occurs in nature.

As used herein, the term “mutant” with respect to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

The term “amino acid” refers to naturally occurring and synthetic amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, γ-carboxyglutamate and O-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e, two) or multimer (i.e., three or more) of single chain polypeptides. The single chain polypeptides of a protein may be joined by a covalent bond, e.g., a disulfide bond, or non-covalent interactions.

The term “conservative substitution,” “conservative mutation,” or “conservatively modified variant” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region, as measured using a sequence comparison algorithm or by manual alignment and visual inspection. In some embodiments, a sequence that has a specified percent identity relative to a reference sequence differs from the reference sequence by one or more conservative substitutions.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a modified Fc polypeptide “corresponds to” an amino acid in SEQ ID NO:1, when the residue aligns with the amino acid in SEQ ID NO:1 when optimally aligned to SEQ ID NO:1. The polypeptide that is aligned to the reference sequence need not be the same length as the reference sequence.

The term “polynucleotide” and “nucleic acid” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

A “binding affinity” as used herein refers to the strength of the non-covalent interaction between two molecules, e.g., a single binding site on a polypeptide and a target, e.g., transferrin receptor, to which it binds. Thus, for example, the term may refer to 1:1 interactions between a polypeptide and its target, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). As used herein, “binding affinity” includes not only formal binding affinities, such as those reflecting 1:1 interactions between a polypeptide and its target, but also apparent affinities for which K_{D}'s are calculated that may reflect avid binding.

As used herein, the term “specifically binds” or “selectively binds” to a target, e.g., TfR, when referring to an engineered TfR-binding polypeptide, TfR-binding peptide, or TfR-binding antibody as described herein, refers to a binding reaction whereby the engineered TfR-binding polypeptide, TfR-binding peptide, or TfR-binding antibody binds to the target with greater affinity, greater avidity, and/or greater duration than it binds to a structurally different target. In typical embodiments, the engineered TfR-binding polypeptide, TfR-binding peptide, or TfR-binding antibody has at least 5-fold, 10-fold, 50-fold, 100-fold, 1,000-fold, 10,000-fold, or greater affinity for a specific target, e.g., TfR, compared to an unrelated target when assayed under the same affinity assay conditions. The term “specific binding,” “specifically binds to,” or “is specific for” a particular target (e.g., TfR), as used herein, can be exhibited, for example, by a molecule having an equilibrium dissociation constant K_{D} for the target to which it binds of, e.g., 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In some embodiments, an engineered TfR-binding polypeptide, TfR-binding peptide, or TfR-binding antibody specifically binds to an epitope on TfR that is conserved among species, (e.g., structurally conserved among species), e.g., conserved between non-human primate and human species (e.g., structurally conserved between non-human primate and human species). In some embodiments, an engineered TfR-binding polypeptide, TfR-binding peptide, or TfR-binding antibody may bind exclusively to a human TfR.

The term “variable region” or “variable domain” refers to a domain in an antibody heavy chain or light chain that is derived from a germline Variable (V) gene, Diversity (D) gene, or Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions.”

The terms “antigen-binding portion” and “antigen-binding fragment” are used interchangeably herein and refer to one or more fragments of an antibody that retains the ability to specifically bind to an antigen via its variable region. Examples of antigen-binding fragments include, but are not limited to, a Fab fragment (a monovalent fragment consisting of the VL, VH, CL, and CH1 domains), a F(ab′)₂ fragment (a bivalent fragment comprising two Fab fragments linked by a disulfide bridge at the hinge region), a single chain Fv (scFv), a disulfide-linked Fv (dsFv), complementarity determining regions (CDRs), a VL (light chain variable region), and a VH (heavy chain variable region).

The following Examples are intended to be non-limiting.

### Example 1: Construction of Fusion Proteins Comprising N-Sulfoglucosamine Sulfohydrolase (SGSH)

### Design and Cloning

SGSH-Fc fusion proteins were designed that contain (i) a first fusion polypeptide where a mature, human SGSH enzyme is fused to a human IgG1 fragment that includes the Fc region (an “SGSH-Fc fusion polypeptide”), and (ii) a second fusion polypeptide where a mature, human SGSH enzyme is fused to a modified human IgG1 fragment which contains mutations in the Fc region that confer transferrin receptor (TfR) binding (a “modified Fc polypeptide”). In particular, SGSH-Fc fusion polypeptides were created in which SGSH fragments were fused to the N-terminus of the human IgG1 Fc region. In some cases, a linker was placed between the SGSH and IgG1 fragments to alleviate any steric hindrance between the two fragments. In all constructs, the signal peptide MGWSCIILFLVATATGAYA (SEQ ID NO: 121) was inserted upstream of the fusion to facilitate secretion, and SGSH was truncated to consist of amino acids R21-L502 (UniProtKB ID—P51688). The fragment of the human IgG1 Fc region used corresponds to amino acids D104-K330 of the sequence in UniProtKB ID P01857 (positions 221-447, EU numbering, which includes 10 amino acids of the hinge (positions 221-230)). The second fusion polypeptide containing SGSH fused to the modified Fc polypeptide was co-transfected with the SGSH-Fc fusion polypeptide to generate heterodimeric fusion proteins with two SGSH enzymes (a “bizyme”). In some constructs, the IgG1 fragments contained additional mutations to facilitate heterodimerization of the two Fc regions. Accordingly, the SGSH-Fc fusion proteins comprising TfR-binding used in the examples are dimers formed by i) an SGSH-Fc fusion polypeptide; and ii) an SGSH-Fc fusion polypeptide that binds TfR comprising a modified Fc polypeptide fused to a second SGSH molecule (a “bizyme”).

Control SGSH-Fc fusion proteins that lack the mutations that confer TfR binding were designed and constructed analogously. An exemplary control SGSH-Fc fusion protein was generated, which comprised a first SGSH-Fc fusion polypeptide having the sequence of any one of SEQ ID NOS:61 and 63 and a second SGSH-Fc fusion polypeptide having the sequence of any one of SEQ ID NOS: 69 and 71. The SGSH-Fc fusion protein may also be further processed during cell culture production, such that the first SGSH-Fc fusion polypeptide has the sequence of SEQ ID NOS:62 or 64 and/or the second SGSH-Fc fusion polypeptide has the sequence of SEQ ID NO:70 or 72. Thus, as used herein, the term SGSH-Fc fusion protein may be used to refer to protein molecules having unprocessed sequences (i.e., SEQ ID NOs:61, 63, 69 and 71); protein molecules comprising one or more processed sequences (i.e., selected from SEQ ID NOs: 62, 64, 70 and 72); or to a mixture comprising processed and unprocessed protein molecules.

An SGSH-Fc fusion polypeptide comprising a mature human SGSH sequence fused to the N-terminus of an IgG1 Fc polypeptide sequence with hole and LALA mutations has the sequence of any one of SEQ ID NOS:61-64. The SGSH enzyme was joined to the Fc polypeptide by a GGGGS linker (SEQ ID NO:8) and the N-terminus of the Fc polypeptide included a portion of an IgG1 hinge region (DKTHTCPPCP; SEQ ID NO:6).

An SGSH-Fc fusion polypeptide comprising a mature human SGSH sequence fused to the N-terminus of an IgG1 Fc polypeptide sequence with hole and LALA mutations has the sequence of any one of SEQ ID NOS:73-76. The SGSH enzyme was joined to the Fc polypeptide by a GS linker and the N-terminus of the Fc polypeptide included a portion of an IgG1 hinge region (DKTHTCPPCP; SEQ ID NO:6).

An SGSH-Fc fusion polypeptide comprising a mature human SGSH sequence fused to the N-terminus of an IgG1 Fc polypeptide sequence with hole and LALA mutations has the sequence of any one of SEQ ID NOS:81-84. The SGSH enzyme was joined to the Fc polypeptide by a (GGGGSGGGGS) linker (SEQ ID NO:9) and the N-terminus of the Fc polypeptide included a portion of an IgG1 hinge region (DKTHTCPPCP; SEQ ID NO:6).

An SGSH-Fc fusion polypeptide comprising a mature human SGSH sequence fused to the N-terminus of an IgG1 Fc polypeptide sequence with hole and LALAPS mutations has the sequence of any one of SEQ ID NOS:65-68. The SGSH enzyme was joined to the Fc polypeptide by a GGGGS linker (SEQ ID NO:8) and the N-terminus of the Fc polypeptide included a portion of an IgG1 hinge region (DKTHTCPPCP; SEQ ID NO:6).

An Fc-SGSH fusion polypeptide comprising a mature human SGSH sequence fused to the C-terminus of an IgG1 Fc polypeptide sequence with hole and LALA mutations has the sequence of any one of SEQ ID NOS:117-118. The SGSH enzyme was joined to the Fc polypeptide by a GGGGS linker (SEQ ID NO:8) and the N-terminus of the Fc polypeptide included a portion of an IgG1 hinge region (DKTHTCPPCP; SEQ ID NO:6).

An SGSH-Fc fusion polypeptide that binds TfR comprising a mature human SGSH sequence fused to the N-terminus of the sequence of a TfR-binding modified Fc polypeptide with knob and LALA mutations has the sequence of any one of SEQ ID NOS:89-92. The SGSH enzyme was joined to the modified Fc polypeptide by a GGGGS linker (SEQ ID NO:8) and the N-terminus of the modified Fc polypeptide included a portion of an IgG1 hinge region (DKTHTCPPCP; SEQ ID NO:6).

An SGSH-Fc fusion polypeptide that binds TfR comprising a mature human SGSH sequence fused to the N-terminus of the sequence of a TfR-binding modified Fc polypeptide with knob and LALA mutations has the sequence of any one of SEQ ID NOS:101-104. The SGSH enzyme was joined to the modified Fc polypeptide by a GS linker and the N-terminus of the modified Fc polypeptide included a portion of an IgG1 hinge region (DKTHTCPPCP; SEQ ID NO:6).

An SGSH-Fc fusion polypeptide that binds TfR comprising a mature human SGSH sequence fused to the N-terminus of the sequence of a TfR-binding modified Fc polypeptide with knob and LALA mutations has the sequence of any one of SEQ ID NOS:109-112. The SGSH enzyme was joined to the modified Fc polypeptide by a GGGGSGGGGS linker (SEQ ID NO:9) and the N-terminus of the modified Fc polypeptide included a portion of an IgG1 hinge region (DKTHTCPPCP; SEQ ID NO:6).

An SGSH-Fc fusion polypeptide that binds TfR comprising a mature human SGSH sequence fused to the N-terminus of the sequence of a TfR-binding modified Fc polypeptide with knob and LALAPS mutations has the sequence of any one of SEQ ID NOS:93-96. The SGSH enzyme was joined to the modified Fc polypeptide by a GGGGS linker (SEQ ID NO:8) and the N-terminus of the modified Fc polypeptide included a portion of an IgG1 hinge region (DKTHTCPPCP; SEQ ID NO:6).

An SGSH-Fc fusion polypeptide that binds TfR comprising a mature human SGSH sequence fused to the C-terminus of the sequence of a TfR-binding modified Fc polypeptide with knob and LALA mutations has the sequence of any one of SEQ ID NOS:119-120. The SGSH enzyme was joined to the modified Fc polypeptide by a GGGGS linker (SEQ ID NO:8) and the N-terminus of the modified Fc polypeptide included a portion of an IgG1 hinge region (DKTHTCPPCP; SEQ ID NO:6).

A first “N-terminal bizyme” SGSH-Fc fusion protein (“ETV:SGSH Bizyme Structure 1”) was generated, which comprised a first SGSH-Fc fusion polypeptide having the sequence of any one of SEQ ID NOS:61 and 63 and a second SGSH-Fc fusion polypeptide that binds TfR having the sequence of any one of SEQ ID NOS: 89 and 91. The SGSH-Fc fusion protein may also be further processed during cell culture production, such that the first SGSH-Fc fusion polypeptide has the sequence of SEQ ID NOS:62 or 64 and/or the second SGSH-Fc fusion polypeptide that binds TfR has the sequence of SEQ ID NO:90 or 92. Thus, as used herein, the term ETV:SGSH Bizyme Structure 1 may be used to refer to protein molecules having unprocessed sequences (i.e., SEQ ID NOs:61, 63, 89 and 91); protein molecules comprising one or more processed sequences (i.e., selected from SEQ ID NOs: 62, 64, 90 and 92); or to a mixture comprising processed and unprocessed protein molecules.

A second “N-terminal bizyme” SGSH-Fc fusion protein (“ETV:SGSH Bizyme Structure 2”) was generated, which comprised a first SGSH-Fc fusion polypeptide having the sequence of any one of SEQ ID NOS:73 and 75 and a second SGSH-Fc fusion polypeptide that binds TfR having the sequence of any one of SEQ ID NOS: 101 and 103. The SGSH-Fc fusion protein may also be further processed during cell culture production, such that the first SGSH-Fc fusion polypeptide has the sequence of SEQ ID NOS:74 or 76 and/or the second SGSH-Fc fusion polypeptide that binds TfR has the sequence of SEQ ID NO:102 or 104. Thus, as used herein, the term ETV:SGSH Bizyme Structure 2 may be used to refer to protein molecules having unprocessed sequences (i.e., SEQ ID NOs:73, 75, 101 and 103); protein molecules comprising one or more processed sequences (i.e., selected from SEQ ID NOs: 74, 76, 102 and 104); or to a mixture comprising processed and unprocessed protein molecules.

A third “N-terminal bizyme” SGSH-Fc fusion protein (“ETV:SGSH Bizyme Structure 3”) was generated, which comprised a first SGSH-Fc fusion polypeptide having the sequence of any one of SEQ ID NOS:81 and 83 and a second SGSH-Fc fusion polypeptide that binds TfR having the sequence of any one of SEQ ID NOS: 109 and 111. The SGSH-Fc fusion protein may also be further processed during cell culture production, such that the first SGSH-Fc fusion polypeptide has the sequence of SEQ ID NOS:82 or 84 and/or the second SGSH-Fc fusion polypeptide that binds TfR has the sequence of SEQ ID NO:110 or 112. Thus, as used herein, the term ETV:SGSH Bizyme Structure 3 may be used to refer to protein molecules having unprocessed sequences (i.e., SEQ ID NOs:81, 83, 109 and 111); protein molecules comprising one or more processed sequences (i.e., selected from SEQ ID NOs: 82, 84, 110 and 112); or to a mixture comprising processed and unprocessed protein molecules.

A fourth “N-terminal bizyme” SGSH-Fc fusion protein (“ETV:SGSH Bizyme Structure 4) was generated, which comprised a first SGSH-Fc fusion polypeptide having the sequence of any one of SEQ ID NOS:65 and 67 and a second SGSH-Fc fusion polypeptide that binds TfR having the sequence of any one of SEQ ID NOS: 93 and 95. The SGSH-Fc fusion protein may also be further processed during cell culture production, such that the first SGSH-Fc fusion polypeptide has the sequence of SEQ ID NO:66 or 68 and/or the second SGSH-Fc fusion polypeptide that binds TfR has the sequence of SEQ ID NO:94 or 96. Thus, as used herein, the term ETV:SGSH Bizyme Structure 4 may be used to refer to protein molecules having unprocessed sequences i.e., SEQ ID NOs:65, 67, 93 and 95); protein molecules comprising one or more processed sequences (i.e., selected from SEQ ID NOs: 66, 68, 94 and 96); or to a mixture comprising processed and unprocessed protein molecules.

A “C-terminal bizyme” SGSH-Fc fusion protein (“ETV:SGSH Bizyme Structure 5) was generated, which comprised a first SGSH-Fc fusion polypeptide having the sequence of any one of SEQ ID NO:117 and 118 and a second SGSH-Fc fusion polypeptide that binds TfR having the sequence of any one of SEQ ID NO:119 and 120. Thus, as used herein, the term ETV:SGSH Bizyme Structure 5 may be used to refer to protein molecules comprising SEQ ID NOs:117 and 119; protein molecules comprising SEQ ID NOs: 118 and 120; or to a mixture comprising SEQ ID NOs: 117 and/or 118 in combination with SEQ ID NOs: 119 and/or 120.

A composition comprising ETV:SGSH (e.g., a structure described above) may be used to refer to a composition comprising protein molecules having unprocessed sequences; protein molecules comprising one or more processed sequences; or to a mixture comprising processed and unprocessed protein molecules.

### Recombinant Protein Expression and Purification

To express recombinant SGSH enzyme fused to an Fc region, ExpiCHO cells (Thermo Fisher Scientific) were transfected with relevant DNA constructs using Expifectamine™ CHO transfection kit according to manufacturer's instructions (Thermo Fisher Scientific). Cells were grown in ExpiCHO™ Expression Medium supplemented with feed as described by the manufacturer's protocol at 37° C., 5% CO₂ and 125 rpm in an orbital shaker (Infors HT Multitron). In brief, logarithmic growing ExpiCHO™ cells were transfected at 6×10⁶ cells/ml density with 0.8 pg of total DNA plasmid per mL of culture volume. Cultures expressing SGSH fusions were co-transfected with a plasmid expressing the cofactor SUMF1 at a plasmid ratio of 5:1 (SGSH:SUMF1). The encoded SUMF1 sequence is described in Genbank NM_182760. After transfection, cells were returned to 37° C. and transfected cultures were supplemented with feed as indicated 18-22 hours post transfection. Transfected cell culture supernatants were harvested 120 hours post transfection by centrifugation at 3,500 rpm from 20 mins. Clarified supernatants were filtered (0.22 μM membrane) and stored at 4° C.

SGSH-Fc fusion proteins with (or without) engineered Fc regions conferring TfR binding were purified from cell culture supernatants using Protein A affinity chromatography. Supernatants were loaded onto a HiTrap MabSelect SuRe Protein A affinity column (GE Healthcare Life Sciences using an Akta Pure System). The column was then washed with 10 column volumes (CVs) of PBS. Bound proteins were eluted using 50 mM citrate/NaOH buffer pH 3.6 containing 150 mM NaCl. Immediately after elution, fractions were neutralized using 1 M Tris pH8 (at a 1:7 dilution). Homogeneity of SGSH-Fc fusions in eluted fractions was assessed by a number of techniques including reducing and non-reducing SDS-PAGE and HPLC-SEC.

### Example 2: Characterization of SGSH Fusion Proteins

### Formylglycine and M6P Content of Fusion Proteins

To characterize certain properties of the SGSH-Fc fusion proteins that impact the enzymatic activity of SGSH and trafficking of the fusion proteins, the formylglycine (fGly) content and mannose-6-phosphate (M6P) content of the SGSH-Fc fusions proteins was evaluated. An ETV:SGSH N-terminal bizyme (Bizyme Structure 1) and a control SGSH-Fc fusion protein (lacking TfR binding), as described in Example 1, were used for the analysis.

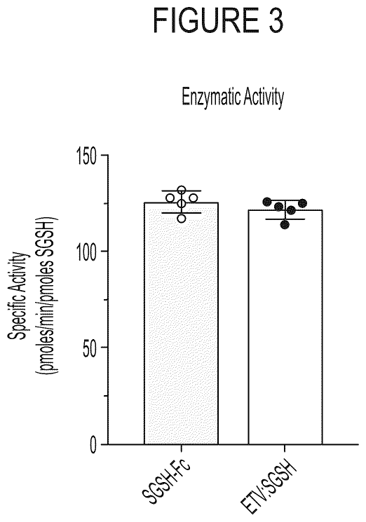

Measurement of fGly content. The identity and quantity of Cys- and FGly-containing peptides were simultaneously assessed by LC-MS/MS. In brief, ˜20 pg of SGSH fusion proteins were reduced with Tris(2-carboxyethyl) phosphine hydrochloride (TCEP·HCl) and alkylated with iodoacetamide then proteolytically digested with Trypsin (70° C. for 2 hours). Formic acid quenched reactions were analyzed by LC-MS/MS. Peptide quantitation analyses were performed by liquid chromatography on UHPLC Vanquish (Thermo Scientific, CA, USA) coupled to UV/Vis and Q Exactive Orbitrap electrospray ionization mass spectrometer (Thermo Scientific, CA, USA). For analysis, samples were injected on a CSH C18 column (Waters Corporation, Milford, Massachusetts, USA) at 40° C. with water with 0.1% formic acid mobile phase. Samples were then subjected to a linear 45 min gradient from %1B to 70% B containing water with 0.1% formic acid (A) and acetonitrile with 0.1% formic acid (B), respectively. The mass spectrometer was operated under Full mass scan at positive mode. Thermo Scientific Freestyle software was used to integrate the peak area or called area under curve (AUC). Three major tryptic peptides containing SGSH cysteine at position 70 (CXPXR motif) as follows were integrated: (1) free Cys, NAFTSVSSCSPSR (SEQ ID NO:127) (2+, m/z 671.806); (2) alkylated carbamidomethyl Cys: NAFTSVSSC(CAM)SPSR (SEQ ID NO:128) (2+, m/z 700.317) and (3) FGly peptide: NAFTSVSS (Fgly) SPSR (SEQ ID NO:129) (2+, m/z 663.810). The calculated % of FGly is based on the AUC of three FGly peptides divided by the AUC sum of FGly and free and alkylated Cys peptides and multiplied by 100. The fGly content of SGSH-Fc and ETV:SGSH was found to be similar to each other (FIG. 2).

Measurement of Mannose-6-phosphate (M6P) content. M6P content in the SGSH-Fc fusion proteins was measured by liquid chromatography-mass spectrometry analysis. Recombinant purified proteins (20 pg) were buffer exchanged into 50 mM ammonium acetate, pH 7.0. Five (5) pg of protein was taken and spiked with stable isotope labeled (SIL) ¹³C₆ mannose-6-phosphate (M6P-IS, Omicronbio Inc, Cat #, MAN-05, 125 ng per sample) as an internal standard. Protein samples were added with 120 μL of a 6.6M trifluoroacetic acid solution and hydrolyzed at 95° C. using heater block for 105 minutes while shaking. Sample dried by nitrogen stream were then washed with acetonitrile (ACN) and dried down again. Final pellets resuspended in 50 μL ACN:water (20:80, v:v) were analyzed by LC-MS/MS. M6P analyses was performed by liquid chromatography on UHPLC Vanquish (Thermo Scientific, CA, USA) coupled to UV/Vis and Q Exactive Orbitrap electrospray ionization mass spectrometer (Thermo Scientific, CA, USA). Samples were injected on a BEH Amide column (Waters) 1.9 μm, 2.1×150 mm, at 60° C. under negative ionization mode in a mobile phase of water with 0.1% formic acid and eluted with a gradient of acetonitrile with 0.1% formic acid. Data was collected using parallel reaction monitoring (PRM) acquisition under negative mode including M6P and M6P internal standard (IS), inclusion time 1.6 to 2.2 min, precursors are 259.0224 (M6P) and 265.0426 (M6P-IS). AUC ratios of M6P/M6P-IS were used to calculate the molecular amount of M6P released from protein and the mol of M6P per mol of protein was obtained. The M6P content of SGSH-Fc and ETV:SGSH is provided in Table 1.

TABLE 1
Mannose-6-phosphate content of fusion proteins

| Molecule | M6P (Mol/Mol) |
|---|---|
| ETV:SGSH | 1.4 |
| SGSH-Fc | 1.2 |

SGSH-Fc Fusion Proteins with Engineered TfR Binding Site Bind to Human TfR

To determine whether SGSH-Fc fusion proteins with engineered TfR binding affects the ability of the modified Fe domain to interact with human TfR, the affinity of ETV:SGSH Bizyme Structure 1 (Example 1) for human TfR was assessed using a Biacore™ surface plasmon resonance assay. Biacore™ Series S CM5 sensor chips were immobilized with anti-human Fab (human Fab capture kit from GE Healthcare). 5 pg/mL of the SGSH-Fc fusion proteins were captured for 1 minute on each flow cell and serial 3-fold dilutions of human apical domain TfR were injected at a flow rate of 30 L/min. Each sample was analyzed with a 3-minute association and a 3-minute dissociation. After each injection, the chip was regenerated using 10 mM glycine-HCl (pH 2.1). Binding response was corrected by subtracting the RU from a flow cell capturing an irrelevant IgG at similar density. Steady-state affinities were obtained by fitting the response at equilibrium against the concentration using Biacore™ T200 Evaluation Software v3.1. Biacore™ analysis established that SGSH-Fc fusion proteins with a TfR-binding site engineered into the Fc region bind to human TfR. In particular, the binding affinity of ETV:SGSH Bizyme Structure 1 for human TfR was determined to be about 230 nM.

SGSH-Fc Fusion Proteins with Engineered TfR Binding Site are Active In Vitro and in Cells

The in vitro and cellular activity of engineered TfR-binding SGSH-Fc fusion proteins were assessed to demonstrate that SGSH maintains its enzymatic activity when fused to the human IgG fragment. The in vitro activity of recombinant SGSH was measured using a two-step fluorometric enzymatic assay using an artificial substrate. Specifically, 20 μL of 1 mM 4-Methylumbelliferyl 2-deoxy-2-sulfamino-a-D-glucopyranoside sodium salt substrate (Carbosynth Limited, #EM06602) diluted in the assay buffer (0.03 M sodium acetate, 0.12 M NaCl, pH 6.5) was mixed with 10-20 μL of 140 nM SGSH. The first reaction was incubated for 17 hr at 37° C. and then terminated with 10 μL of 0.2 M phosphate-citrate buffer, pH 6.7. Next, the second reaction was initiated by adding 10 μL (0.5 U) of yeast α-Glucosidase (Sigma, #G0660-750UN), incubated for 24 hr at 37° C., and stopped with the addition of 100 μL of 0.5 M sodium carbonate buffer, pH 10.3. Fluorescence of the reaction solution was then measured (excitation at 365 nm and emission at 450 nm). A 4-Methylumbelliferone standard curve was fit by linear regression to calculate the amount of product and verified as less than 10% of total substrate cleavage. Specific activity (fmol product/min/pmol SGSH) was calculated by dividing the amount of product by the reaction time and molar amount of SGSH.

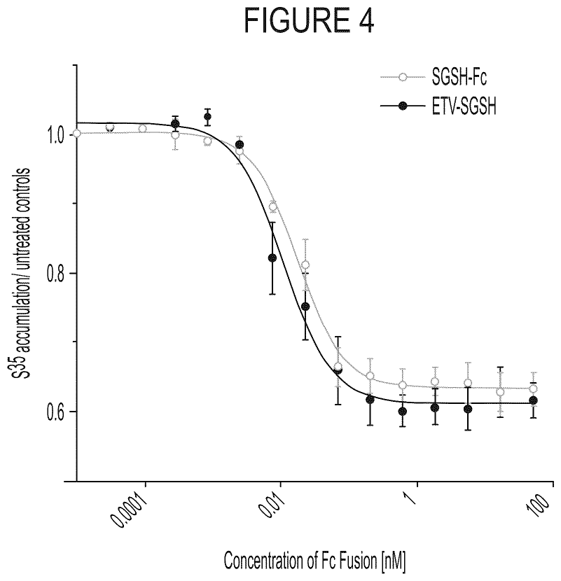

The in vitro enzymatic activity assay demonstrated that SGSH-Fc fusion proteins were active and were similar between Fc-SGSH (control; Example 1) and ETV:SGSH (Bizyme Structure 1; Example 1) (FIG. 3).

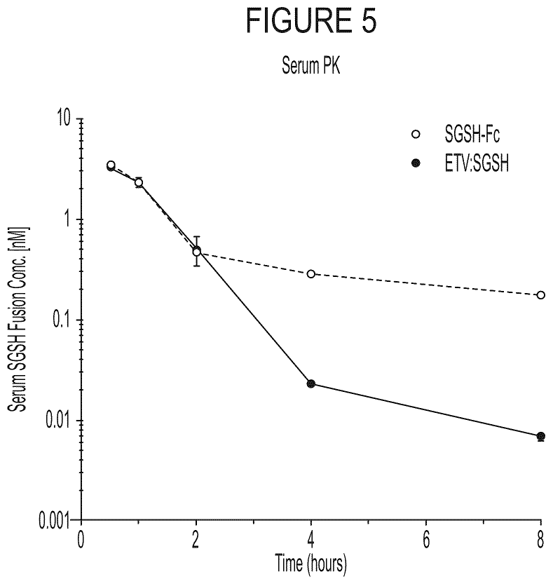

The cellular activity of SGSH-Fc fusion proteins was also examined in fibroblasts from MPS IIIA patients and healthy controls using a ³⁵S pulse-chase assay, in which ³⁵S is integrated into newly-synthesized GAGs, as previously described (Boado et al., Mol. Pharm. 11(8): 2928-2934 [2014]). MPS IIIA patient fibroblasts lack SGSH activity, leading to an increased accumulation of ³⁵S signal. The SGSH-Fc fusion proteins, including ETV:SGSH (Bizyme Structure 1), were highly efficacious in MPS IIIA patient-derived cells, displaying a low picomolar cellular EC₅₀ for reducing the accumulation of S³⁵-labeled material (FIG. 4).

SGSH-Fc Fusion Proteins with Engineered TfR Binding Site Show Improved Brain Delivery in a Mouse Model of MPSHIII

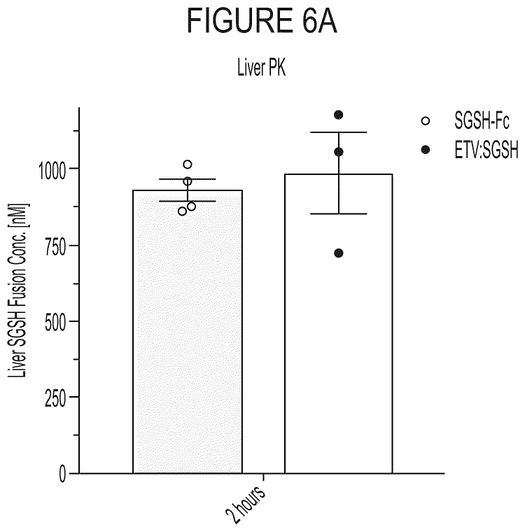

To determine whether TfR-binding SGSH-Fc fusion proteins showed improved brain delivery compared to a control SGSH-Fc fusion protein, human TfR knock-in (TfR^{mu/hu} KI) mice were dosed with 40 mg/kg of the TfR-binding SGSH-Fc fusion protein ETV:SGSH (Bizyme Structure 1) or a control SGSH-Fc fusion protein lacking the mutations that confer TfR binding (“SGSH:Fc”) (see, Example 1), and the concentration of the SGSH-Fc fusion protein in liver and brain was measured using a sandwich ELISA-based assay at 2 and 8 hours post-dose. The SGSH-Fc fusion proteins that were used in the analysis are described above and were prepared in accordance with Example 1 (referred to herein as ETV:SGSH (Bizyme Structure 1) and control SGSH-Fc). A polyclonal donkey anti-human IgG capture antibody, specific for the Fc fragment (Jackson ImmunoResearch, #709-006-098) was coated onto a 384-well MaxiSorp™ plate (Thermo Scientific #464718) overnight. The plate was blocked with 5% BSA and then incubated with diluted serum, brain and liver lysates. Next, an HRP-conjugated polyclonal goat anti-human IgG specific for the Fc fragment (Jackson ImmunoResearch, #109-036-098) was added for detection. The plates were developed using TMB substrate, stopped with sulfuric acid, and the absorbance at 450 nm measured on a BioTek plate reader. The standard curves were the individual constructs from 2000-2.74 pM in a 3-fold dilution series and were fit using a five-parameter logistic curve. TfR^{mu/hu} KI mice were generated as described in International Patent Publication No. WO 2018/152285 using CRISPR/Cas9 technology to express human Tfrc apical domain within the murine Tfrc gene; the resulting chimeric TfR was expressed in vivo under the control of the endogenous promoter. The results are illustrated in FIGS. 5-7.

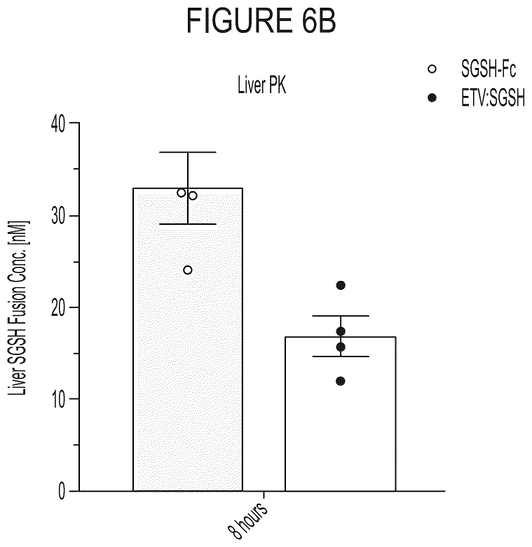

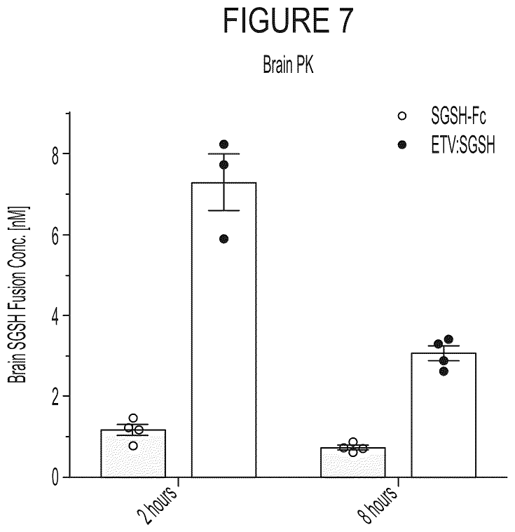

Administration of the TfR-binding SGSH-Fc fusion protein led to an approximately 6-fold increase in brain uptake relative to the control SGSH-Fc fusion protein at 2 hours and an approximately 4-fold increase in brain concentration at 8 hours post-dose (FIG. 7). Accumulation of the fusion proteins in the liver were equivalent for both ETV:SGSH and SGSH:Fc at 2 hours but decreased considerably (approximately 30-fold) at 8 hours post-dose, with ETV:SGSH exhibiting lower levels compared to SGSH:Fc (FIG. 6). The concentration of fusion proteins in serum was measured using a sandwich ELISA-based assay as described above at 0.5, 1, 2, 4, and 8 hours post-dose. Serum PK was equivalent for both ETV:SGSH and SGSH:Fc at 2 hours but ETV:SGSH exhibited lower levels compared to SGSH:Fc between 2 and 8 hours post-dose (FIG. 5). While the brain levels of TfR-binding SGSH-Fc fusion proteins remained elevated for 8 hours compared to the control SGSH:Fc fusion protein, the faster peripheral clearance may account for the decrease in brain and liver concentrations from 2 to 8 hours post-dose. Together, these data demonstrate that the interaction of the TfR-binding SGSH-Fc fusion proteins with TfR generally maintains peripheral distribution while significantly improving brain exposure.

### Intravenous Administration of ETV:SGSH Reduces GAGs in the Brain

To examine whether the improved brain exposure observed with the TfR-binding SGSH-Fc fusion proteins described above and prepared in accordance with Example 1 (referred to herein as ETV:SGSH) produced a corresponding reduction of accumulated substrates in the brain, a mouse model containing a sulfamidase mutation that harbors the human TfR apical domain knocked into the murine TfR was generated (referred to herein as Sgsh^{mps3a}×TfR^{mu/hu} KI mice, or alternatively, as SGSH^{D31N}; TfR^{mu/hu} KI mice). Sgsh^{mps3a} mice containing a novel sulfamidase mutation, D31N, were obtained from The Jackson Laboratories (JAX stock #003780). Briefly, TfR^{mu/hu} KI male mice were bred to female Sgsh^{mps3a} heterozygous mice to generate mice homozygous for the Sgsh^{mps3a} mutation in a TfR^{mu/hu} KI homozygous background. Mice used in this study were mixed sex and housed under a 12 hour light-dark cycle with ad libitum access to food (LabDiet JL irradiated 6F) and water.

Sgsh^{mps3a}×TfR^{mu/hu} KI mice were administered a single dose of 40 mg/kg body weight of ETV:SGSH (Bizyme Structure 1) or SGSH-Fc via intravenous injection and pharmacodynamic responses were assessed (see, Example 1 for fusion proteins). In particular, the effect of peripheral administration of ETV:SGSH on liver, brain and CSF HS levels in Sgsh^{mps3a}×TfR^{mu/hu} KI mice was determined using 3-month-old Sgsh^{mps3a}×TfR^{mu/hu} KI mice injected intravenously (i.v.) with saline, SGSH-Fc (40 mg/kg body weight), or ETV:SGSH (40 mg/kg body weight) (n=8/group). 3-month-old littermate TfR^{mu/hu} KI mice, injected i.v. with saline were used as controls. All animals were sacrificed 7 days post single dose except for a subset of Sgsh^{mps3a}×TfR^{mu/hu} KI mice injected with ETV:SGSH (n=4) that were sacrificed 3 days post single dose. Serum, CSF, liver, and brain were collected and flash-frozen on dry ice.

Heparan sulfate-derived disaccharides were measured in vivo using LC-MS/MS-based methods as described below. Briefly, all tissues and fluids were collected and then immediately frozen and stored at −80° C. Tissue aliquots (50 mg) were homogenized in water (750 μL) using the Qiagen TissueLyzer II for 3 minutes at 30 Hz. Homogenate was transferred to a 96-well deep plate and sonicated using a 96-tip sonicator (Q Sonica) for 10×1 second pulses. Sonicated homogenates were spun at 2,500×g for 30 minutes at 4° C. to pellet cell debris. The resulting lysate was transferred to a clean 96-well deep plate, and a BCA was performed to quantify total protein. Heparan sulfate (HS) in the samples were digested to their corresponding disaccharides prior to LC-MS/MS analysis. 10 μg of total protein lysate or 3 μl of CSF was incubated with Heparinases I, II, and III in digestion buffer [111 mM NH40Ac, 0.11 mM CaOAc, 2 mM DTT, pH 7.0] for 3 hours with shaking at 30° C. in a PCR plate. After 3 hours, EDTA and 20 ng of the internal standard D4UA-2S-GlcNCOEt-6S (HD009, Iduron Ltd, Manchester, UK) were added to each sample and the mixture was boiled at 95° C. for 10 minutes to inactivate the enzymes. The digested samples were spun at 3,364×g for 5 minutes and supernatants were transferred to a cellulose acetate filter plate (Millipore, MSUN03010) and spun at 3,364×g for 5 minutes. The resulting eluent was mixed with equal parts of acetonitrile in glass vials and analyzed by mass spectrometry as below.

Quantification of HS derived disaccharides in fluids and tissues was performed by liquid chromatography (Shimadzu Nexera X2 system, Shimadzu Scientific Instrument, Columbia, MD, USA) coupled to electrospray mass spectrometry (Sciex 6500+QTRAP, Sciex, Framingham, MA, USA). For each analysis, sample was injected on a ACQUITY UPLC BEH Amide 1.7 mm, 2.1×150 mm column (Waters Corporation, Milford, MA, USA) using a flow rate of 0.4 mL/minute with a column temperature of 50° C. Mobile phase A consisted of water with 10 mM ammonium formate and 0.1% formic acid, and mobile phase B consisted of acetonitrile with 0.1% formic acid. The gradient was programmed as follows: 0.0-1.0 minutes at 85% B, 1.0-5.0 minutes from 85% B to 50% B, 5.0-6.0 minutes 50% B to 85% B, 6-8.0 minutes hold at 85% B. Electrospray ionization was performed in negative-ionization mode applying the following settings: curtain gas at 30; collision gas at medium; ion spray voltage at −4500; temperature at 450° C.; ion source Gas 1 at 50; and ion source Gas 2 at 60. Data acquisition was performed using Analyst 1.6.3 (Sciex) in multiple reaction monitoring mode (MRM) with the following settings: dwell time at 30 msec; collision energy at −30; declustering potential at −80; entrance potential at −10; collision cell exit potential at −10. Individual disaccharide species were identified based on their retention times and MRM transitions using commercially available reference standards (Iduron Ltd). The following disaccharide transitions were monitored: D0A0 (HS), m/z 378.1>87.0; D0S0 (HS), m/z 416.1>138.0; D4UA-2S-GlcNCOEt-6S (internal standard) m/z 472.0>97.0. Disaccharide amounts were normalized to total protein levels as measured by a BCA assay, or to the volume of body fluid used per sample.

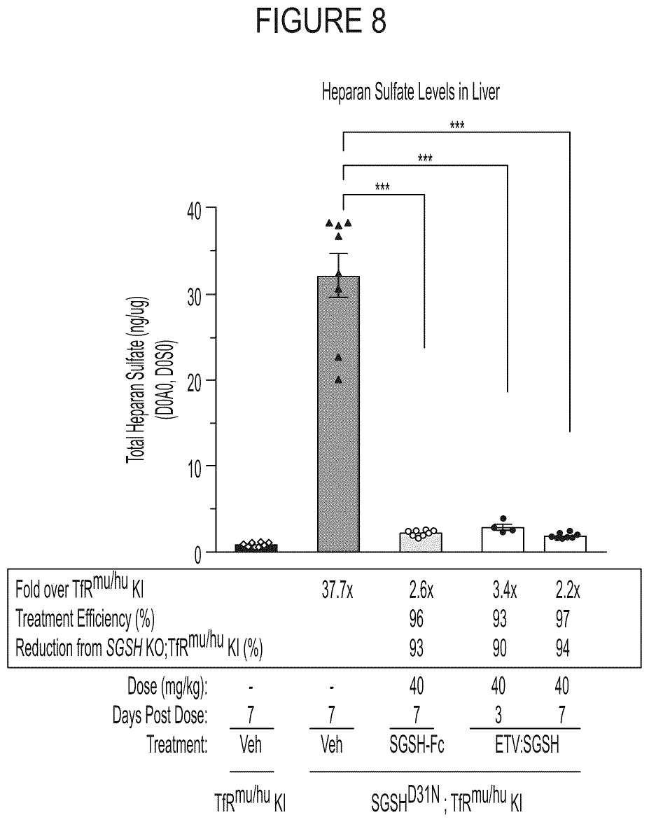

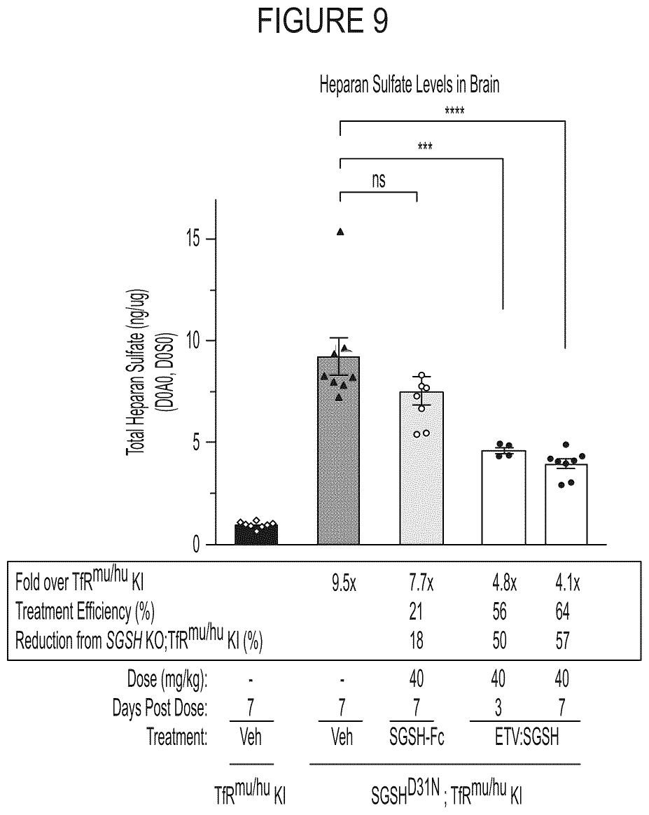

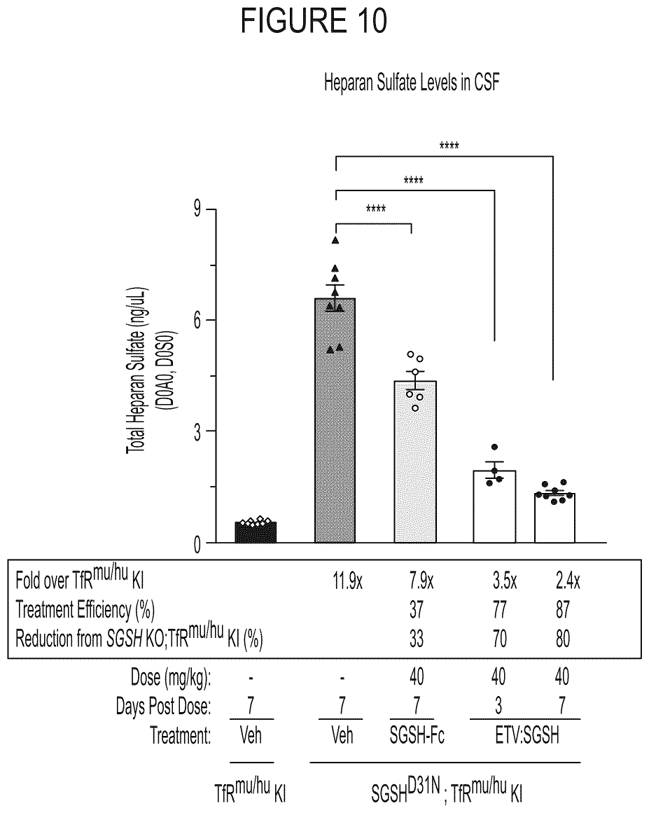

To determine whether ETV:SGSH reduces substrate levels in the brain, HS levels were assessed in Sgsh^{mps3a}×TfR^{mu/hu} KI mice after a single dose of enzyme. SGSH-Fc was ineffective at lowering brain HS levels following a single dose (FIG. 9). ETV:SGSH, however, reduced brain HS levels by approximately 50% and 57% at 3 days and 7 days following a single dose, respectively (FIG. 9). This led to a concomitant reduction of CSF HS levels by approximately 70% and 80% at 3 days and 7 days following a single dose, respectively (FIG. 10). Both molecules effectively lowered HS levels in liver after one week (FIG. 8), demonstrating that TfR binding does not negatively impact pharmacodynamic responses in these tissues. The data in FIGS. 8-10 is represented by mean+/−standard error of the mean (*p<0.05, **p<0.01, ***p<0.001, ****p<0.0001, ns=not significant). Together, these data demonstrate that ETV:SGSH significantly increases brain exposure of enzyme and robustly reduces substrate accumulation in both the periphery and CNS.

### Example 3: Product Quality Attributes of ETV:SGSH Bizyme Structures

Different bizyme structures of ETV:SGSH fusion proteins were evaluated in terms of product quality. For this study, ETV:SGSH Bizyme Structure 1 (Example 1) was compared to a structure having a different TfR binding Fc region (ETV:SGSH Bizyme Structure 6, described below). Both structures were prepared as described in Example 1, with additional purification steps as described below.

### Results

Measured human TfR affinities for Bizyme Structure 1 and Bizyme Structure 6 were comparable (K_{D} of about 290 nM vs. about 245 nM, respectively).

The expression titer for Bizyme Structure 1 was determined to be about 30-40 mg/L, whereas expression titer for Bizyme Structure 6 measured slightly less (about 12-23 mg/L).

Post protein A chromatography purification recovery of both Bizyme Structure 1 and Bizyme Structure 6 was evaluated. Analysis of post-protein A pools of both Bizyme Structure 1 and Bizyme Structure 6 illustrated about 50-60% purity (as measured by HPLC-SEC) with intact ETV structure (maintenance of modified Fc dimer comprising knob and hole pair) of at least about 80%. The post-protein A pools of both bizyme structures underwent hydrophobic interaction chromatography (HIC) for further polishing (described below). Post-HIC pools of Bizyme Structure 1 achieved purity levels of >95% (as measured by HPLC-SEC) with intact ETV structure of >90%, while post-HIC pools of Bizyme Structure 6 achieved purity levels of about 85% (as measured by HPLC-SEC) with intact ETV structure of >90%. In order to achieve higher purity levels (>90%) for Bizyme Structure 6, additional purification steps are needed, which could result in reduced yield and recovery of protein post-purification.

Accordingly, Bizyme Structure 1 and its P329S variant (Bizyme Structure 4) were identified as preferred structures for moving to larger-scale production.

### Experimental Methods

A sixth “N-terminal bizyme” SGSH-Fc fusion protein (“ETV:SGSH Bizyme Structure 6”) was generated, which comprised a first SGSH-Fc fusion polypeptide having the sequence of any one of SEQ ID NOS:61 and 63 and a second SGSH-Fc fusion polypeptide that binds TfR having the sequence of any one of SEQ ID NOS: 122 and 124. The SGSH-Fc fusion protein may also be further processed during cell culture production, such that the first SGSH-Fc fusion polypeptide has the sequence of SEQ ID NO:62 or 64 and/or the second SGSH-Fc fusion polypeptide that binds TfR has the sequence of SEQ ID NO:123 or 125. Thus, as used herein, the term ETV:SGSH Bizyme Structure 6 may be used to refer to protein molecules having unprocessed sequences (i.e., SEQ ID NOs:61, 63, 122 and 124); protein molecules comprising one or more processed sequences (i.e., selected from SEQ ID NOs: 62, 64, 123 and 125); or to a mixture comprising processed and unprocessed protein molecules.

ETV:SGSH Bizyme Structure 1 and ETV:SGSH Bizyme Structure 6 were expressed and purified as described in Example 1 with the following modification: Neutralization of the pooled protein fractions eluted from the Protein A affinity column was carried out with 1M Tris pH 8.0 to target pH of 6.0. The neutralized Protein A pool was then conditioned with 1 M Sodium Citrate to a final concentration of 0.6 M Sodium Citrate. The pooled fractions were loaded onto a ButylHP Hydrophobic Interaction Chromatography (HIC) column, washed with 0.6 M Sodium Citrate (pH 6.0), and eluted via (i) a 50% step gradient of 0.6 M Sodium Citrate (pH 6.0) to WFI over 10 CVs, followed by (ii) a 100% step gradient of 0.6 M Sodium Citrate (pH 6.0) to WFI over 5 CVs.

Homogeneity of ETV:SGSH fusion proteins in the eluted fractions was assessed by a number of techniques including reducing and non-reducing SDS-PAGE and HPLC-SEC.

Affinity for human TfR was measured as described in Example 2.

### Example 4: Intravenous Administration of Different Bizyme Structures for ETV:SGSH Achieves Comparable Reduction of GAGs in the Brain

Different bizyme structures of ETV:SGSH fusion proteins were evaluated in terms of effect on brain GAG levels in a mouse model of MPS III. For this study, ETV:SGSH Bizyme Structure 1 was compared to a corresponding structure that contains the P329S mutation in the Fc region (ETV:SGSH Bizyme Structure 4).

### Results

The bizyme structures were analyzed for formylglycine (fGly) content, mannose-6-phosphate (M6P) content, and human TfR affinity using methods described in Example 2. Table 2 provides the analysis results for each bizyme structure. Post-HIC pooled fractions of Bizyme Structure 1 and Bizyme Structure 4 achieved purity levels of >95% (as measured by HPLC-SEC) with intact ETV structure of >90%.

TABLE 2
ETV:SGSH Protein Characteristics

| | | M6P | TfR affinity |
|---|---|---|---|
| Molecule | fGly | (mol/mol) | (K_{D}) |
| Bizyme Structure 1 | 98% | 4.05 | 290 nM |
| Bizyme Structure 4 | 99% | 2.93 | 340 nM |

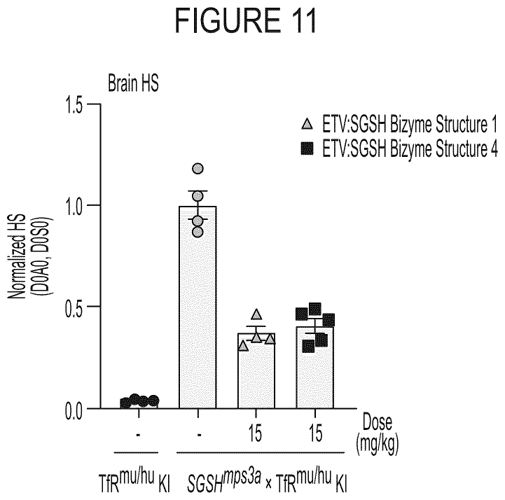

To determine whether the ETV:SGSH structures reduced substrate levels in the brain, HS levels were assessed in Sgsh^{mps3a}×TfR^{mu/hu} KI mice after a single dose of ETV:SGSH protein. Both ETV:SGSH Bizyme Structure 1 and ETV:SGSH Bizyme Structure 4 reduced brain HS levels by approximately 63% and 59% at 7 days following a single dose, respectively (FIG. 11). The data in FIG. 11 is represented by mean+/−standard error of the mean. This data demonstrates that both bizyme structures of ETV:SGSH robustly reduced substrate accumulation in the brain. Brain uptake of both bizyme structures at 7 days post-dose were detectable and quantified as greater than 0.5 nM in brain tissue sampled from each cohort.

### Experimental Methods

ETV:SGSH Bizyme Structure 1 was expressed and purified as described in Example 3.

ETV:SGSH Bizyme Structure 4 was expressed from stable CHO cell lines that were transfected with relevant DNA constructs and selected by evaluation of expression titer, stability, and activity of the expressed and purified proteins. Briefly, CHO-K1 GS knockout cell line (Horizon Discovery) was transfected with relevant DNA constructs (co-transfection of plasmids coding for fusion protein and SUMF1), followed by selection to generate a stable cell line expressing the gene of interest. The cell line was then subjected to fed batch production commercial CHO cell culture medium (e.g., BalanCD CHO medium (Irvine Scientific), optionally supplemented with BalanCD CHO Feed 4 (Irvine Scientific)). The culture was maintained at 37° C. for 5 days, followed by a temperature shift to 32° C. Upon harvest at day 12, the cell culture was centrifuged, and the supernatant was sterile-filtered through a commercial (0.8 μm/0.2 μm membrane filter) and stored at 4° C. The fusion protein was purified from cell culture supernatants using Protein A affinity and Hydrophobic Interaction chromatography. Supernatants were loaded onto a preparative scale MabSelect SuRe LX Protein A affinity column (GE Healthcare Life Sciences using an Akta Pure System). The column was then washed with 2 column volumes (CVs) of PBS, followed by 4 CVs of 0.4 M Potassium Phosphate pH 7.0, followed by 3 CVs of PBS. Bound proteins were eluted using 50 mM citrate/NaOH buffer pH 3.7. Immediately after elution, fractions were neutralized using 1.5 M Tris pH11 to a target pH of 6.0. Neutralized Protein A pools were adjusted with 1 M Sodium Citrate pH 6.0, at a ratio of 1:1.3, prior to Hydrophobic Interaction chromatography. The adjusted Protein A Pool was loaded onto a ButylHP Hydrophobic Interaction Chromatography (HIC) column, washed with 0.6 M Sodium Citrate pH 6.0, and then eluted via a 20-55% gradient from 0.6 M Sodium Citrate to WFJ over 25 CVs. Homogeneity of the fusion protein in eluted fractions was assessed by a number of techniques including reducing and non-reducing SDS-PAGE and HPLC-SEC.

The fusion proteins were analyzed for formylglycine (fGly) content, M6u content, and TfR affinity using methods described in Example 2.

Sgsh^{mps3a}×TfR^{mu/hu} KI mice (Example 2) were administered a single dose of ETV: SGSH Bizyme Structure 1 or ETV:SGSH Bizyme Structure 4 via intravenous injection, and brain exposure and pharmacodynamic responses were assessed. The effect of peripheral administration of the ETV:SGSH bizyme structures on brain HS levels in Sgsh^{mps3a}×TfR^{mu/hu} KI mice was determined using 9-month-old Sgsh^{mps3a}×TfR^{mu/hu} KI mice injected intravenously (i.v.) with saline, ETV:SGSH Bizyme Structure 1 (15 mg/kg body weight), or ETV: SGSH Bizyme Structure 4 (15 mg/kg body weight) (n=4-5/group). Nine-month-old littermate TfR^{mu/hu} KI mice (non-NIPS III mice) injected i.v. with saline were used as controls. All animals were sacrificed 7 days post single dose. Brain tissue was collected and flash-frozen on dry ice. Brain uptake of ETV: SGSH and heparan sulfate-derived disaccharides were measured as described in Example 2.

Informal Sequence Listing

| SEQ ID | | |
|---|---|---|
| NO: | Sequence | Description |
| 1 | APELLGGPSVFLFPPKPKDTLMISRTPEVTCVVVDVSHEDPEVKFNWYVD | Wild-type human |
| | GVEVHNAKTKPREEQYNSTYRVVSVLTVLHQDWLNGKEYKCKVSNKALPA | Fc sequence |
| | PIEKTISKAKGQPREPQVYTLPPSRDELTKNQVSLTCLVKGFYPSDIAVE | positions 231-447 |
| | WESNGQPENNYKTTPPVLDSDGSFFLYSKLTVDKSRWQQGNVFSCSVMHE | EU index |
| | ALHNHYTQKSLSLSPGK | numbering |
| 2 | APELLGGPSVFLFPPKPKDTLMISRTPEVTCVVVDVSHEDPEVKFNWYVD | Wild-type human |
| | GVEVHNAKTKPREEQYNSTYRVVSVLTVLHQDWLNGKEYKCKVSNKALPA | Fc sequence |
| | PIEKTISKAKGQPREPQVYTLPPSRDELTKNQVSLTCLVKGFYPSDIAVE | positions 231-446 |
| | WESNGQPENNYKTTPPVLDSDGSFFLYSKLTVDKSRWQQGNVFSCSVMHE | EU index |
| | ALHNHYTQKSLSLSPG | numbering |
| 3 | APELLGGPSVFLFPPKPKDTLMISRTPEVTCVVVDVSHEDPEVKFNWYVD | CH2 domain |
| | GVEVHNAKTKPREEQYNSTYRVVSVLTVLHQDWLNGKEYKCKVSNKALPA | sequence |
| | PIEKTISKAK | positions 231-340 |
| | | EU index |
| | | numbering |
| 4 | GQPREPQVYTLPPSRDELTKNQVSLTCLVKGFYPSDIAVEWESNGQPENN | CH3 domain |
| | YKTTPPVLDSDGSFFLYSKLTVDKSRWQQGNVFSCSVMHEALHNHYTQKS | sequence |
| | LSLSPGK | Positions 341-447 |
| | | EU index |
| | | numbering |
| 5 | EPKSCDKTHTCPPCP | Human IgG1 hinge |
| | | amino acid |
| | | sequence |
| 6 | DKTHTCPPCP | Portion of human |
| | | IgG1 hinge |
| | | sequence |
| | GS | GS linker |
| 8 | GGGGS | Glycine-rich |
| | | linker |
| 9 | GGGGSGGGGS | Glycine-rich |
| | | linker |
| 10 | MMDQARSAFSNLFGGEPLSYTRFSLARQVDGDNSHVEMKLAVDEEENADN | Human transferrin |
| | NTKANVTKPKRCSGSICYGTIAVIVFFLIGFMIGYLGYCKGVEPKTECER | receptor protein |
| | LAGTESPVREEPGEDFPAARRLYWDDLKRKLSEKLDSTDFTGTIKLLNEN | 1 (TFR1) |
| | SYVPREAGSQKDENLALYVENQFREFKLSKVWRDQHFVKIQVKDSAQNSV | |
| | IIVDKNGRLVYLVENPGGYVAYSKAATVTGKLVHANFGTKKDFEDLYTPV | |
| | NGSIVIVRAGKITFAEKVANAESLNAIGVLIYMDQTKFPIVNAELSFFGH | |
| | AHLGTGDPYTPGFPSFNHTQFPPSRSSGLPNIPVQTISRAAAEKLFGNME | |
| | GDCPSDWKTDSTCRMVTSESKNVKLTVSNVLKEIKILNIFGVIKGFVEPD | |
| | HYVVVGAQRDAWGPGAAKSGVGTALLLKLAQMFSDMVLKDGFQPSRSIIF | |
| | ASWSAGDFGSVGATEWLEGYLSSLHLKAFTYINLDKAVLGTSNFKVSASP | |
| | LLYTLIEKTMQNVKHPVTGQFLYQDSNWASKVEKLTLDNAAFPFLAYSGI | |
| | PAVSFCFCEDTDYPYLGTTMDTYKELIERIPELNKVARAAAEVAGQFVIK | |
| | LTHDVELNLDYERYNSQLLSFVRDLNQYRADIKEMGLSLQWLYSARGDFF | |
| | RATSRLTTDFGNAEKTDRFVMKKLNDRVMRVEYHFLSPYVSPKESPFRHV | |
| | FWGSGSHTLPALLENLKLRKQNNGAFNETLFRNQLALATWTIQGAANALS | |
| | GDVWDIDNEF | |
| 11 | NSVIIVDKNGRLVYLVENPGGYVAYSKAATVTGKLVHANFGTKKDFEDLY | Human TfR apical |
| | TPVNGSIVIVRAGKITFAEKVANAESLNAIGVLIYMDQTKFPIVNAELSF | domain |
| | FGHAHLGTGDPYTPGFPSFNHTQFPPSRSSGLPNIPVQTISRAAAEKLFG | |
| | NMEGDCPSDWKTDSTCRMVTSESKNVKLTVS | |
| 12 | APELLGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | hole mutations |
| | PIEKTISKAKGQPREPQVYTLPPSRDELTKNQVSLSCAVKGFYPSDIAVE | |
| | WESNGQPENNYKTTPPVLDSDGSFFLVSKLTVDKSRWQQGNVFSCSVMHE | |
| | ALHNHYTQKSLSLSPGK | |
| 13 | APELLGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | hole mutations |
| | PIEKTISKAKGQPREPQVYTLPPSRDELTKNQVSLSCAVKGFYPSDIAVE | |
| | WESNGQPENNYKTTPPVLDSDGSFFLVSKLTVDKSRWQQGNVFSCSVMHE | |
| | ALHNHYTQKSLSLSPG | |
| 14 | APEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | hole and LALA |
| | PIEKTISKAKGQPREPQVYTLPPSRDELTKNQVSLSCAVKGFYPSDIAVE | mutations |
| | WESNGQPENNYKTTPPVLDSDGSFFLVSKLTVDKSRWQQGNVFSCSVMHE | |
| | ALHNHYTQKSLSLSPGK | |
| 15 | APEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | hole and LALA |
| | PIEKTISKAKGQPREPQVYTLPPSRDELTKNQVSLSCAVKGFYPSDIAVE | mutations |
| | WESNGQPENNYKTTPPVLDSDGSFFLVSKLTVDKSRWQQGNVFSCSVMHE | |
| | ALHNHYTQKSLSLSPG | |
| 16 | APEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALGA | hole and LALAPG |
| | PIEKTISKAKGQPREPQVYTLPPSRDELTKNQVSLSCAVKGFYPSDIAVE | mutations |
| | WESNGQPENNYKTTPPVLDSDGSFFLVSKLTVDKSRWQQGNVFSCSVMHE | |
| | ALHNHYTQKSLSLSPGK | |
| 17 | APEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALGA | hole and LALAPG |
| | PIEKTISKAKGQPREPQVYTLPPSRDELTKNQVSLSCAVKGFYPSDIAVE | mutations |
| | WESNGQPENNYKTTPPVLDSDGSFFLVSKLTVDKSRWQQGNVFSCSVMHE | |
| | ALHNHYTQKSLSLSPG | |
| 18 | APEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALSA | hole and LALAPS |
| | PIEKTISKAKGQPREPQVYTLPPSRDELTKNQVSLSCAVKGFYPSDIAVE | mutations |
| | WESNGQPENNYKTTPPVLDSDGSFFLVSKLTVDKSRWQQGNVFSCSVMHE | |
| | ALHNHYTQKSLSLSPGK | |
| 19 | APEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALSA | hole and LALAPS |
| | PIEKTISKAKGQPREPQVYTLPPSRDELTKNQVSLSCAVKGFYPSDIAVE | mutations |
| | WESNGQPENNYKTTPPVLDSDGSFFLVSKLTVDKSRWQQGNVFSCSVMHE | |
| | ALHNHYTQKSLSLSPG | |
| 24 | APELLGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | knob mutation |
| | PIEKTISKAKGQPREPQVYTLPPSRDELTKNQVSLWCLVKGFYPSDIAVE | |
| | WESNGQPENNYKTTPPVLDSDGSFFLYSKLTVDKSRWQQGNVFSCSVMHE | |
| | ALHNHYTQKSLSLSPGK | |
| 25 | APELLGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | knob mutation |
| | PIEKTISKAKGQPREPQVYTLPPSRDELTKNQVSLWCLVKGFYPSDIAVE | |
| | WESNGQPENNYKTTPPVLDSDGSFFLYSKLTVDKSRWQQGNVFSCSVMHE | |
| | ALHNHYTQKSLSLSPG | |
| 26 | APEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | knob and LALA |
| | PIEKTISKAKGQPREPQVYTLPPSRDELTKNQVSLWCLVKGFYPSDIAVE | mutations |
| | WESNGQPENNYKTTPPVLDSDGSFFLYSKLTVDKSRWQQGNVFSCSVMHE | |
| | ALHNHYTQKSLSLSPGK | |
| 27 | APEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | knob and LALA |
| | PIEKTISKAKGQPREPQVYTLPPSRDELTKNQVSLWCLVKGFYPSDIAVE | mutations |
| | WESNGQPENNYKTTPPVLDSDGSFFLYSKLTVDKSRWQQGNVFSCSVMHE | |
| | ALHNHYTQKSLSLSPG | |
| 28 | DKTHTCPPCPAPEAAGGPSVFLFPPKPKDTLMISRTPEVTCVVVDVSHED | Fc sequence with |
| | PEVKFNWYVDGVEVHNAKTKPREEQYNSTYRVVSVLTVLHQDWLNGKEYK | hole and LALA |
| | CKVSNKALPAPIEKTISKAKGQPREPQVYTLPPSRDELTKNQVSLSCAVK | mutations and |
| | GFYPSDIAVEWESNGQPENNYKTTPPVLDSDGSFFLVSKLTVDKSRWQQG | portion of human |
| | NVFSCSVMHEALHNHYTQKSLSLSPGK | IgG1 hinge |
| | | sequence |
| 29 | DKTHTCPPCPAPEAAGGPSVFLFPPKPKDTLMISRTPEVTCVVVDVSHED | Fc sequence with |
| | PEVKFNWYVDGVEVHNAKTKPREEQYNSTYRVVSVLTVLHQDWLNGKEYK | hole and LALA |
| | CKVSNKALPAPIEKTISKAKGQPREPQVYTLPPSRDELTKNQVSLSCAVK | mutations and |
| | GFYPSDIAVEWESNGQPENNYKTTPPVLDSDGSFFLVSKLTVDKSRWQQG | portion of human |
| | NVFSCSVMHEALHNHYTQKSLSLSPG | IgG1 hinge |
| | | sequence |
| 30 | DKTHTCPPCPAPEAAGGPSVFLFPPKPKDTLMISRTPEVTCVVVDVSHED | Fc sequence with |
| | PEVKFNWYVDGVEVHNAKTKPREEQYNSTYRVVSVLTVLHQDWLNGKEYK | hole and LALAPS |
| | CKVSNKALSAPIEKTISKAKGQPREPQVYTLPPSRDELTKNQVSLSCAVK | mutations and |
| | GFYPSDIAVEWESNGQPENNYKTTPPVLDSDGSFFLVSKLTVDKSRWQQG | portion of human |
| | NVFSCSVMHEALHNHYTQKSLSLSPGK | IgG1 hinge |
| | | sequence |
| 31 | DKTHTCPPCPAPEAAGGPSVFLFPPKPKDTLMISRTPEVTCVVVDVSHED | Fc sequence with |
| | PEVKFNWYVDGVEVHNAKTKPREEQYNSTYRVVSVLTVLHQDWLNGKEYK | hole and LALAPS |
| | CKVSNKALSAPIEKTISKAKGQPREPQVYTLPPSRDELTKNQVSLSCAVK | mutations and |
| | GFYPSDIAVEWESNGQPENNYKTTPPVLDSDGSFFLVSKLTVDKSRWQQG | portion of human |
| | NVFSCSVMHEALHNHYTQKSLSLSPG | IgG1 hinge |
| | | sequence |
| 32 | APELLGGPSVFLFPPKPKDTLMISRTPEVTCVWDVSHEDPEVKFNWYVD | Clone |
| | GVEVHNAKTKPREEQYNSTYRVVSVLTVLHQDWLNGKEYKCKVSNKALPA | CH3C.35.23.2 |
| | PIEKTISKAKGQPREPQVYTLPPSRDELTKNQVSLTCLVKGFYPSDIAVE | |
| | WESYGTEWANYKTTPPVLDSDGSFFLYSKLTVTKEEWQQGFVFSCSVMHE | |
| | ALHNHYTQKSLSLSPGK | |
| 33 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 |
| | PIEKTISKAKGQPREPQVYTLPPSRDELTKNQVSLTCLVKGFYPSDIAVE | |
| | WESYGTEWANYKTTPPVLDSDGSFFLYSKLTVTKEEWQQGFVFSCSVMHE | |
| | ALHNHYTQKSLSLSPG | |
| 34 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob mutation |
| | WESYGTEWANYKTTPPVLDSDGSFFLYSKLTVTKEEWQQGFVFSCSVMHE | |
| | ALHNHYTQKSLSLSPGK | |
| 35 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob mutation |
| | WESYGTEWANYKTTPPVLDSDGSFFLYSKLTVTKEEWQQGFVFSCSVMHE | |
| | ALHNHYTQKSLSLSPG | |
| 36 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob and LALA |
| | WESYGTEWANYKTTPPVLDSDGSFFLYSKLTVTKEEWQQGFVFSCSVMHE | mutations |
| | ALHNHYTQKSLSLSPGK | |
| 37 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob and LALA |
| | WESYGTEWANYKTTPPVLDSDGSFFLYSKLTVTKEEWQQGFVFSCSVMHE | mutations |
| | ALHNHYTQKSLSLSPG | |
| 38 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.2 with |
| | PIEKTISKAKGQPREPQVYTLPPSRDELTKNQVSLWCLVKGFYPSDIAVE | knob and LALAPG |
| | WESYGTEWANYKTTPPVLDSDGSFFLYSKLTVTKEEWQQGFVFSCSVMHE | mutations |
| | ALHNHYTQKSLSLSPGK | |
| 39 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.2 with |
| | PIEKTISKAKGQPREPQVYTLPPSRDELTKNQVSLWCLVKGFYPSDIAVE | knob and LALAPG |
| | WESYGTEWANYKTTPPVLDSDGSFFLYSKLTVTKEEWQQGFVFSCSVMHE | mutations |
| | ALHNHYTQKSLSLSPG | |
| 40 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALSA | CH3C.35.23.2 with |
| | PIEKTISKAKGQPREPQVYTLPPSRDELTKNQVSLWCLVKGFYPSDIAVE | knob and LALAPS |
| | WESYGTEWANYKTTPPVLDSDGSFFLYSKLTVTKEEWQQGFVFSCSVMHE | mutations |
| | ALHNHYTQKSLSLSPGK | |
| 41 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALSA | CH3C.35.23.2 with |
| | PIEKTISKAKGQPREPQVYTLPPSRDELTKNQVSLWCLVKGFYPSDIAVE | knob and LALAPS |
| | WESYGTEWANYKTTPPVLDSDGSFFLYSKLTVTKEEWQQGFVFSCSVMHE | mutations |
| | ALHNHYTQKSLSLSPG | |
| 48 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLSCAVKGFYPSDIAVE | hole mutations |
| | WESYGTEWANYKTTPPVLDSDGSFFLVSKLTVTKEEWQQGFVFSCSVMHE | |
| | ALHNHYTQKSLSLSPGK | |
| 49 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLSCAVKGFYPSDIAVE | hole mutations |
| | WESYGTEWANYKTTPPVLDSDGSFFLVSKLTVTKEEWQQGFVFSCSVMHE | |
| | ALHNHYTQKSLSLSPG | |
| 50 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLSCAVKGFYPSDIAVE | hole and LALA |
| | WESYGTEWANYKTTPPVLDSDGSFFLVSKLTVTKEEWQQGFVFSCSVMHE | mutations |
| | ALHNHYTQKSLSLSPGK | |
| 51 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLSCAVKGFYPSDIAVE | hole and LALA |
| | WESYGTEWANYKTTPPVLDSDGSFFLVSKLTVTKEEWQQGFVFSCSVMHE | mutations |
| | ALHNHYTQKSLSLSPG | |
| 52 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.2 with |
| | PIEKTISKAKGQPREPQVYTLPPSRDELTKNQVSLSCAVKGFYPSDIAVE | hole and LALAPG |
| | WESYGTEWANYKTTPPVLDSDGSFFLVSKLTVTKEEWQQGFVFSCSVMHE | mutations |
| | ALHNHYTQKSLSLSPGK | |
| 53 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.2 with |
| | PIEKTISKAKGQPREPQVYTLPPSRDELTKNQVSLSCAVKGFYPSDIAVE | hole and LALAPG |
| | WESYGTEWANYKTTPPVLDSDGSFFLVSKLTVTKEEWQQGFVFSCSVMHE | mutations |
| | ALHNHYTQKSLSLSPG | |
| 54 | DKTHTCPPCPAPEAAGGPSVFLFPPKPKDTLMISRTPEVTCVVVDVSHED | Clone |
| | PEVKFNWYVDGVEVHNAKTKPREEQYNSTYRVVSVLTVLHQDWLNGKEYK | CH3C.35.23.2 with |
| | CKVSNKALPAPIEKTISKAKGQPREPQVYTLPPSRDELTKNQVSLWCLVK | knob and LALA |
| | GFYPSDIAVEWESYGTEWANYKTTPPVLDSDGSFFLYSKLTVTKEEWQQG | mutations and |
| | FVFSCSVMHEALHNHYTQKSLSLSPGK | portion of human |
| | | IgG1 hinge |
| | | sequence |
| 55 | DKTHTCPPCPAPEAAGGPSVFLFPPKPKDTLMISRTPEVTCVVVDVSHED | Clone |
| | PEVKFNWYVDGVEVHNAKTKPREEQYNSTYRVVSVLTVLHQDWLNGKEYK | CH3C.35.23.2 with |
| | CKVSNKALPAPIEKTISKAKGQPREPQVYTLPPSRDELTKNQVSLWCLVK | knob and LALA |
| | GFYPSDIAVEWESYGTEWANYKTTPPVLDSDGSFFLYSKLTVTKEEWQQG | mutations and |
| | FVFSCSVMHEALHNHYTQKSLSLSPG | portion of human |
| | | IgG1 hinge |
| | | sequence |
| 56 | DKTHTCPPCPAPEAAGGPSVFLFPPKPKDTLMISRTPEVTCVVVDVSHED | Clone |
| | PEVKFNWYVDGVEVHNAKTKPREEQYNSTYRVVSVLTVLHQDWLNGKEYK | CH3C.35.23.2 with |
| | CKVSNKALSAPIEKTISKAKGQPREPQVYTLPPSRDELTKNQVSLWCLVK | knob and LALAPS |
| | GFYPSDIAVEWESYGTEWANYKTTPPVLDSDGSFFLYSKLTVTKEEWQQG | mutations and |
| | FVFSCSVMHEALHNHYTQKSLSLSPGK | portion of human |
| | | IgG1 hinge |
| | | sequence |
| 57 | DKTHTCPPCPAPEAAGGPSVFLFPPKPKDTLMISRTPEVTCVVVDVSHED | Clone |
| | PEVKFNWYVDGVEVHNAKTKPREEQYNSTYRVVSVLTVLHQDWLNGKEYK | CH3C.35.23.2 with |
| | CKVSNKALSAPIEKTISKAKGQPREPQVYTLPPSRDELTKNQVSLWCLVK | knob and LALAPS |
| | GFYPSDIAVEWESYGTEWANYKTTPPVLDSDGSFFLYSKLTVTKEEWQQG | mutations and |
| | FVFSCSVMHEALHNHYTQKSLSLSPG | portion of human |
| | | IgG1 hinge |
| | | sequence |
| 58 | MSCPVPACCALLLVLGLCRARPRNALLLLADDGGFESGAYNNSAIATPHL | Full-length human |
| | DALARRSLLFRNAFTSVSSCSPSRASLLTGLPQHQNGMYGLHQDVHHFNS | sulfoglucosamine |
| | FDKVRSLPLLLSQAGVRTGIIGKKHVGPETVYPFDFAYTEENGSVLQVGR | sulfohydrolase |
| | NITRIKLLVRKFLQTQDDRPFFLYVAFHDPHRCGHSQPQYGTFCEKFGNG | polypeptide |
| | ESGMGRIPDWTPQAYDPLDVLVPYFVPNTPAARADLAAQYTTVGRMDQGV | sequence |
| | GLVLQELRDAGVLNDTLVIFTSDNGIPFPSGRTNLYWPGTAEPLLVSSPE | |
| | HPKRWGQVSEAYVSLLDLTPTILDWFSIPYPSYAIFGSKTIHLTGRSLLP | |
| | ALEAEPLWATVFGSQSHHEVTMSYPMRSVQHRHFRLVHNLNFKMPFPIDQ | |
| | DFYVSPTFQDLLNRTTAGQPTGWYKDLRHYYYRARWELYDRSRDPHETQN | |
| | LATDPRFAQLLEMLRDQLAKWQWETHDPWVCAPDGVLEEKLSPQCQPLHN | |
| | EL | |
| 59 | RPRNALLLLADDGGFESGAYNNSAIATPHLDALARRSLLFRNAFTSVSSC | Mature human |
| | SPSRASLLTGLPQHQNGMYGLHQDVHHFNSFDKVRSLPLLLSQAGVRTGI | sulfoglucosamine |
| | IGKKHVGPETVYPFDFAYTEENGSVLQVGRNITRIKLLVRKFLQTQDDRP | sulfohydrolase |
| | FFLYVAFHDPHRCGHSQPQYGTFCEKFGNGESGMGRIPDWTPQAYDPLDV | polypeptide |
| | LVPYFVPNTPAARADLAAQYTTVGRMDQGVGLVLQELRDAGVLNDTLVIF | sequence |
| | TSDNGIPFPSGRTNLYWPGTAEPLLVSSPEHPKRWGQVSEAYVSLLDLTP | |
| | TILDWFSIPYPSYAIFGSKTIHLTGRSLLPALEAEPLWATVFGSQSHHEV | |
| | TMSYPMRSVQHRHFRLVHNLNFKMPFPIDQDFYVSPTFQDLLNRTTAGQP | |
| | TGWYKDLRHYYYRARWELYDRSRDPHETQNLATDPRFAQLLEMLRDQLAK | |
| | WQWETHDPWVCAPDGVLEEKLSPQCQPLHNEL | |
| 60 | RPRNALLLLADDGGFESGAYNNSAIATPHLDALARRSLLFRNAFTSVSS | Mature human |
| | fGSPSRASLLTGLPQHQNGMYGLHQDVHHFNSFDKVRSLPLLLSQAGVRT | sulfoglucosamine |
| | GIIGKKHVGPETVYPFDFAYTEENGSVLQVGRNITRIKLLVRKFLQTQDD | sulfohydrolase |
| | RPFFLYVAFHDPHRCGHSQPQYGTFCEKFGNGESGMGRIPDWTPQAYDPL | polypeptide |
| | DVLVPYFVPNTPAARADLAAQYTTVGRMDQGVGLVLQELRDAGVLNDTLV | sequence |
| | IFTSDNGIPFPSGRTNLYWPGTAEPLLVSSPEHPKRWGQVSEAYVSLLDL | (formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) |
| | AKWQWETHDPWVCAPDGVLEEKLSPQCQPLHNEL | |
| 61 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | G4S linker (SEQ |
| | TILDWFSIPYPSYAIFGSKTIHLTGRSLLPALEAEPLWATVFGSQSHHEV | ID NO: 8) fused |
| | TMSYPMRSVQHRHFRLVHNLNFKMPFPIDQDFYVSPTFQDLLNRTTAGQP | to the N-terminus |
| | TGWYKDLRHYYYRARWELYDRSRDPHETQNLATDPRFAQLLEMLRDQLAK | of an Fc sequence |
| | WQWETHDPWVCAPDGVLEEKLSPQCQPLHNELGGGGSDKTHTCPPCPAPE | with hole and |
| | AAGGPSVFLFPPKPKDTLMISRTPEVTCVVVDVSHEDPEVKFNWYVDGVE | LALA mutations |
| | VHNAKTKPREEQYNSTYRVVSVLTVLHQDWLNGKEYKCKVSNKALPAPIE | |
| | KTISKAKGQPREPQVYTLPPSRDELTKNQVSLSCAVKGFYPSDIAVEWES | |
| | NGQPENNYKTTPPVLDSDGSFFLVSKLTVDKSRWQQGNVFSCSVMHEALH | |
| | NHYTQKSLSLSPGK | |
| 62 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | G4S linker (SEQ |
| | TILDWFSIPYPSYAIFGSKTIHLTGRSLLPALEAEPLWATVFGSQSHHEV | ID NO: 8) fused |
| | TMSYPMRSVQHRHFRLVHNLNFKMPFPIDQDFYVSPTFQDLLNRTTAGQP | to the N-terminus |
| | TGWYKDLRHYYYRARWELYDRSRDPHETQNLATDPRFAQLLEMLRDQLAK | of an Fc sequence |
| | WQWETHDPWVCAPDGVLEEKLSPQCQPLHNELGGGGSDKTHTCPPCPAPE | with hole and |
| | AAGGPSVFLFPPKPKDTLMISRTPEVTCVVVDVSHEDPEVKFNWYVDGVE | LALA mutations |
| | VHNAKTKPREEQYNSTYRVVSVLTVLHQDWLNGKEYKCKVSNKALPAPIE | |
| | KTISKAKGQPREPQVYTLPPSRDELTKNQVSLSCAVKGFYPSDIAVEWES | |
| | NGQPENNYKTTPPVLDSDGSFFLVSKLTVDKSRWQQGNVFSCSVMHEALH | |
| | NHYTQKSLSLSPG | |
| 63 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSDKTHTCPPCPA | G4S linker (SEQ |
| | PEAAGGPSVFLFPPKPKDTLMISRTPEVTCVVVDVSHEDPEVKFNWYVDG | ID NO: 8) fused |
| | VEVHNAKTKPREEQYNSTYRVVSVLTVLHQDWLNGKEYKCKVSNKALPAP | to the N-terminus |
| | IEKTISKAKGQPREPQVYTLPPSRDELTKNQVSLSCAVKGFYPSDIAVEW | of an Fc sequence |
| | ESNGQPENNYKTTPPVLDSDGSFFLVSKLTVDKSRWQQGNVFSCSVMHEA | with hole and |
| | LHNHYTQKSLSLSPGK | LALA mutations |
| 64 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSDKTHTCPPCPA | G4S linker (SEQ |
| | PEAAGGPSVFLFPPKPKDTLMISRTPEVTCVVVDVSHEDPEVKFNWYVDG | ID NO: 8) fused |
| | VEVHNAKTKPREEQYNSTYRVVSVLTVLHQDWLNGKEYKCKVSNKALPAP | to the N-terminus |
| | IEKTISKAKGQPREPQVYTLPPSRDELTKNQVSLSCAVKGFYPSDIAVEW | of an Fc sequence |
| | ESNGQPENNYKTTPPVLDSDGSFFLVSKLTVDKSRWQQGNVFSCSVMHEA | with hole and |
| | LHNHYTQKSLSLSPG | LALA mutations |
| 65 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | G4S linker (SEQ |
| | TILDWFSIPYPSYAIFGSKTIHLTGRSLLPALEAEPLWATVFGSQSHHEV | ID NO: 8) fused |
| | TMSYPMRSVQHRHFRLVHNLNFKMPFPIDQDFYVSPTFQDLLNRTTAGQP | to the N-terminus |
| | TGWYKDLRHYYYRARWELYDRSRDPHETQNLATDPRFAQLLEMLRDQLAK | of an Fc sequence |
| | WQWETHDPWVCAPDGVLEEKLSPQCQPLHNELGGGGSDKTHTCPPCPAPE | with hole and |
| | AAGGPSVFLFPPKPKDTLMISRTPEVTCVVVDVSHEDPEVKFNWYVDGVE | LALAPS mutations |
| | VHNAKTKPREEQYNSTYRVVSVLTVLHQDWLNGKEYKCKVSNKALSAPIE | |
| | KTISKAKGQPREPQVYTLPPSRDELTKNQVSLSCAVKGFYPSDIAVEWES | |
| | NGQPENNYKTTPPVLDSDGSFFLVSKLTVDKSRWQQGNVFSCSVMHEALH | |
| | NHYTQKSLSLSPGK | |
| 66 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | G4S linker (SEQ |
| | TILDWFSIPYPSYAIFGSKTIHLTGRSLLPALEAEPLWATVFGSQSHHEV | ID NO: 8) fused |
| | TMSYPMRSVQHRHFRLVHNLNFKMPFPIDQDFYVSPTFQDLLNRTTAGQP | to the N-terminus |
| | TGWYKDLRHYYYRARWELYDRSRDPHETQNLATDPRFAQLLEMLRDQLAK | of an Fc sequence |
| | WQWETHDPWVCAPDGVLEEKLSPQCQPLHNELGGGGSDKTHTCPPCPAPE | with hole and |
| | AAGGPSVFLFPPKPKDTLMISRTPEVTCVVVDVSHEDPEVKFNWYVDGVE | LALAPS mutations |
| | VHNAKTKPREEQYNSTYRVVSVLTVLHQDWLNGKEYKCKVSNKALSAPIE | |
| | KTISKAKGQPREPQVYTLPPSRDELTKNQVSLSCAVKGFYPSDIAVEWES | |
| | NGQPENNYKTTPPVLDSDGSFFLVSKLTVDKSRWQQGNVFSCSVMHEALH | |
| | NHYTQKSLSLSPG | |
| 67 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSDKTHTCPPCPA | G4S linker (SEQ |
| | PEAAGGPSVFLFPPKPKDTLMISRTPEVTCVVVDVSHEDPEVKFNWYVDG | ID NO: 8) fused |
| | VEVHNAKTKPREEQYNSTYRVVSVLTVLHQDWLNGKEYKCKVSNKALSAP | to the N-terminus |
| | IEKTISKAKGQPREPQVYTLPPSRDELTKNQVSLSCAVKGFYPSDIAVEW | of an Fc sequence |
| | ESNGQPENNYKTTPPVLDSDGSFFLVSKLTVDKSRWQQGNVFSCSVMHEA | with hole and |
| | LHNHYTQKSLSLSPGK | LALAPS mutations |
| 68 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSDKTHTCPPCPA | G4S linker (SEQ |
| | PEAAGGPSVFLFPPKPKDTLMISRTPEVTCVVVDVSHEDPEVKFNWYVDG | ID NO: 8) fused |
| | VEVHNAKTKPREEQYNSTYRVVSVLTVLHQDWLNGKEYKCKVSNKALSAP | to the N-terminus |
| | IEKTISKAKGQPREPQVYTLPPSRDELTKNQVSLSCAVKGFYPSDIAVEW | of an Fc sequence |
| | ESNGQPENNYKTTPPVLDSDGSFFLVSKLTVDKSRWQQGNVFSCSVMHEA | with hole and |
| | LHNHYTQKSLSLSPG | LALAPS mutations |
| 69 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | G4S linker (SEQ |
| | TILDWFSIPYPSYAIFGSKTIHLTGRSLLPALEAEPLWATVFGSQSHHEV | ID NO: 8) fused |
| | TMSYPMRSVQHRHFRLVHNLNFKMPFPIDQDFYVSPTFQDLLNRTTAGQP | to the N-terminus |
| | TGWYKDLRHYYYRARWELYDRSRDPHETQNLATDPRFAQLLEMLRDQLAK | of an Fc sequence |
| | WQWETHDPWVCAPDGVLEEKLSPQCQPLHNELGGGGSDKTHTCPPCPAPE | with knob and |
| | AAGGPSVFLFPPKPKDTLMISRTPEVTCVVVDVSHEDPEVKFNWYVDGVE | LALA mutations |
| | VHNAKTKPREEQYNSTYRVVSVLTVLHQDWLNGKEYKCKVSNKALPAPIE | |
| | KTISKAKGQPREPQVYTLPPSRDELTKNQVSLWCLVKGFYPSDIAVEWES | |
| | NGQPENNYKTTPPVLDSDGSFFLYSKLTVDKSRWQQGNVFSCSVMHEALH | |
| | NHYTQKSLSLSPGK | |
| 70 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | G4S linker (SEQ |
| | TILDWFSIPYPSYAIFGSKTIHLTGRSLLPALEAEPLWATVFGSQSHHEV | ID NO: 8) fused |
| | TMSYPMRSVQHRHFRLVHNLNFKMPFPIDQDFYVSPTFQDLLNRTTAGQP | to the N-terminus |
| | TGWYKDLRHYYYRARWELYDRSRDPHETQNLATDPRFAQLLEMLRDQLAK | of an Fc sequence |
| | WQWETHDPWVCAPDGVLEEKLSPQCQPLHNELGGGGSDKTHTCPPCPAPE | with knob and |
| | AAGGPSVFLFPPKPKDTLMISRTPEVTCVVVDVSHEDPEVKFNWYVDGVE | LALA mutations |
| | VHNAKTKPREEQYNSTYRVVSVLTVLHQDWLNGKEYKCKVSNKALPAPIE | |
| | KTISKAKGQPREPQVYTLPPSRDELTKNQVSLWCLVKGFYPSDIAVEWES | |
| | NGQPENNYKTTPPVLDSDGSFFLYSKLTVDKSRWQQGNVFSCSVMHEALH | |
| | NHYTQKSLSLSPG | |
| 71 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSDKTHTCPPCPA | G4S linker (SEQ |
| | PEAAGGPSVFLFPPKPKDTLMISRTPEVTCVVVDVSHEDPEVKFNWYVDG | ID NO: 8) fused |
| | VEVHNAKTKPREEQYNSTYRVVSVLTVLHQDWLNGKEYKCKVSNKALPAP | to the N-terminus |
| | IEKTISKAKGQPREPQVYTLPPSRDELTKNQVSLWCLVKGFYPSDIAVEW | of an Fc sequence |
| | ESNGQPENNYKTTPPVLDSDGSFFLYSKLTVDKSRWQQGNVFSCSVMHEA | with knob and |
| | LHNHYTQKSLSLSPGK | LALA mutations |
| 72 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSDKTHTCPPCPA | G4S linker (SEQ |
| | PEAAGGPSVFLFPPKPKDTLMISRTPEVTCVVVDVSHEDPEVKFNWYVDG | ID NO: 8) fused |
| | VEVHNAKTKPREEQYNSTYRVVSVLTVLHQDWLNGKEYKCKVSNKALPAP | to the N-terminus |
| | IEKTISKAKGQPREPQVYTLPPSRDELTKNQVSLWCLVKGFYPSDIAVEW | of an Fc sequence |
| | ESNGQPENNYKTTPPVLDSDGSFFLYSKLTVDKSRWQQGNVFSCSVMHEA | with knob and |
| | LHNHYTQKSLSLSPG | LALA mutations |
| 73 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | GS linker |
| | TILDWFSIPYPSYAIFGSKTIHLTGRSLLPALEAEPLWATVFGSQSHHEV | fused to |
| | TMSYPMRSVQHRHFRLVHNLNFKMPFPIDQDFYVSPTFQDLLNRTTAGQP | the N-terminus of |
| | TGWYKDLRHYYYRARWELYDRSRDPHETQNLATDPRFAQLLEMLRDQLAK | an Fc sequence |
| | WQWETHDPWVCAPDGVLEEKLSPQCQPLHNELGSDKTHTCPPCPAPEAAG | with hole and |
| | GPSVFLFPPKPKDTLMISRTPEVTCVVVDVSHEDPEVKFNWYVDGVEVHN | LALA mutations |
| | AKTKPREEQYNSTYRVVSVLTVLHQDWLNGKEYKCKVSNKALPAPIEKTI | |
| | SKAKGQPREPQVYTLPPSRDELTKNQVSLSCAVKGFYPSDIAVEWESNGQ | |
| | PENNYKTTPPVLDSDGSFFLVSKLTVDKSRWQQGNVFSCSVMHEALHNHY | |
| | TQKSLSLSPGK | |
| 74 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | GS linker |
| | TILDWFSIPYPSYAIFGSKTIHLTGRSLLPALEAEPLWATVFGSQSHHEV | fused to |
| | TMSYPMRSVQHRHFRLVHNLNFKMPFPIDQDFYVSPTFQDLLNRTTAGQP | the N-terminus of |
| | TGWYKDLRHYYYRARWELYDRSRDPHETQNLATDPRFAQLLEMLRDQLAK | an Fc sequence |
| | WQWETHDPWVCAPDGVLEEKLSPQCQPLHNELGSDKTHTCPPCPAPEAAG | with hole and |
| | GPSVFLFPPKPKDTLMISRTPEVTCVVVDVSHEDPEVKFNWYVDGVEVHN | LALA mutations |
| | AKTKPREEQYNSTYRVVSVLTVLHQDWLNGKEYKCKVSNKALPAPIEKTI | |
| | SKAKGQPREPQVYTLPPSRDELTKNQVSLSCAVKGFYPSDIAVEWESNGQ | |
| | PENNYKTTPPVLDSDGSFFLVSKLTVDKSRWQQGNVFSCSVMHEALHNHY | |
| | TQKSLSLSPG | |
| 75 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGSDKTHTCPPCPAPEA | GS linker |
| | AGGPSVFLFPPKPKDTLMISRTPEVTCVVVDVSHEDPEVKFNWYVDGVEV | fused to |
| | HNAKTKPREEQYNSTYRVVSVLTVLHQDWLNGKEYKCKVSNKALPAPIEK | the N-terminus of |
| | TISKAKGQPREPQVYTLPPSRDELTKNQVSLSCAVKGFYPSDIAVEWESN | an Fc sequence |
| | GQPENNYKTTPPVLDSDGSFFLVSKLTVDKSRWQQGNVFSCSVMHEALHN | with hole and |
| | HYTQKSLSLSPGK | LALA mutations |
| 76 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGSDKTHTCPPCPAPEA | GS linker |
| | AGGPSVFLFPPKPKDTLMISRTPEVTCVVVDVSHEDPEVKFNWYVDGVEV | fused to |
| | HNAKTKPREEQYNSTYRVVSVLTVLHQDWLNGKEYKCKVSNKALPAPIEK | the N-terminus of |
| | TISKAKGQPREPQVYTLPPSRDELTKNQVSLSCAVKGFYPSDIAVEWESN | an Fc sequence |
| | GQPENNYKTTPPVLDSDGSFFLVSKLTVDKSRWQQGNVFSCSVMHEALHN | with hole and |
| | HYTQKSLSLSPG | LALA mutations |
| 77 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | GS linker |
| | TILDWFSIPYPSYAIFGSKTIHLTGRSLLPALEAEPLWATVFGSQSHHEV | fused to |
| | TMSYPMRSVQHRHFRLVHNLNFKMPFPIDQDFYVSPTFQDLLNRTTAGQP | the N-terminus of |
| | TGWYKDLRHYYYRARWELYDRSRDPHETQNLATDPRFAQLLEMLRDQLAK | an Fc sequence |
| | WQWETHDPWVCAPDGVLEEKLSPQCQPLHNELGSDKTHTCPPCPAPEAAG | with knob and |
| | GPSVFLFPPKPKDTLMISRTPEVTCVVVDVSHEDPEVKFNWYVDGVEVHN | LALA mutations |
| | AKTKPREEQYNSTYRWSVLTVLHQDWLNGKEYKCKVSNKALPAPIEKTI | |
| | SKAKGQPREPQVYTLPPSRDELTKNQVSLWCLVKGFYPSDIAVEWESNGQ | |
| | PENNYKTTPPVLDSDGSFFLYSKLTVDKSRWQQGNVFSCSVMHEALHNHY | |
| | TQKSLSLSPGK | |
| 78 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | GS linker |
| | TILDWFSIPYPSYAIFGSKTIHLTGRSLLPALEAEPLWATVFGSQSHHEV | fused to |
| | TMSYPMRSVQHRHFRLVHNLNFKMPFPIDQDFYVSPTFQDLLNRTTAGQP | the N-terminus of |
| | TGWYKDLRHYYYRARWELYDRSRDPHETQNLATDPRFAQLLEMLRDQLAK | an Fc sequence |
| | WQWETHDPWVCAPDGVLEEKLSPQCQPLHNELGSDKTHTCPPCPAPEAAG | with knob and |
| | GPSVFLFPPKPKDTLMISRTPEVTCVVVDVSHEDPEVKFNWYVDGVEVHN | LALA mutations |
| | AKTKPREEQYNSTYRVVSVLTVLHQDWLNGKEYKCKVSNKALPAPIEKTI | |
| | SKAKGQPREPQVYTLPPSRDELTKNQVSLWCLVKGFYPSDIAVEWESNGQ | |
| | PENNYKTTPPVLDSDGSFFLYSKLTVDKSRWQQGNVFSCSVMHEALHNHY | |
| | TQKSLSLSPG | |
| 79 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGSDKTHTCPPCPAPEA | GS linker |
| | AGGPSVFLFPPKPKDTLMISRTPEVTCVVVDVSHEDPEVKFNWYVDGVEV | fused to |
| | HNAKTKPREEQYNSTYRVVSVLTVLHQDWLNGKEYKCKVSNKALPAPIEK | the N-terminus of |
| | TISKAKGQPREPQVYTLPPSRDELTKNQVSLWCLVKGFYPSDIAVEWESN | an Fc sequence |
| | GQPENNYKTTPPVLDSDGSFFLYSKLTVDKSRWQQGNVFSCSVMHEALHN | with knob and |
| | HYTQKSLSLSPGK | LALA mutations |
| 80 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGSDKTHTCPPCPAPEA | GS linker |
| | AGGPSVFLFPPKPKDTLMISRTPEVTCVVVDVSHEDPEVKFNWYVDGVEV | fused to |
| | HNAKTKPREEQYNSTYRVVSVLTVLHQDWLNGKEYKCKVSNKALPAPIEK | the N-terminus of |
| | TISKAKGQPREPQVYTLPPSRDELTKNQVSLWCLVKGFYPSDIAVEWESN | an Fc sequence |
| | GQPENNYKTTPPVLDSDGSFFLYSKLTVDKSRWQQGNVFSCSVMHEALHN | with knob and |
| | HYTQKSLSLSPG | LALA mutations |
| 81 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | (G₄S)₂ linker |
| | TILDWFSIPYPSYAIFGSKTIHLTGRSLLPALEAEPLWATVFGSQSHHEV | (SEQ ID NO: 9) |
| | TMSYPMRSVQHRHFRLVHNLNFKMPFPIDQDFYVSPTFQDLLNRTTAGQP | fused to the N- |
| | TGWYKDLRHYYYRARWELYDRSRDPHETQNLATDPRFAQLLEMLRDQLAK | terminus of an Fc |
| | WQWETHDPWVCAPDGVLEEKLSPQCQPLHNELGGGGSGGGGSDKTHTCPP | sequence with |
| | CPAPEAAGGPSVFLFPPKPKDTLMISRTPEVTCVVVDVSHEDPEVKFNWY | hole and LALA |
| | VDGVEVHNAKTKPREEQYNSTYRVVSVLTVLHQDWLNGKEYKCKVSNKAL | mutations |
| | PAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSCSVM | |
| | HEALHNHYTQKSLSLSPGK | |
| 82 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | (G₄S)₂ linker |
| | TILDWFSIPYPSYAIFGSKTIHLTGRSLLPALEAEPLWATVFGSQSHHEV | (SEQ ID NO: 9) |
| | TMSYPMRSVQHRHFRLVHNLNFKMPFPIDQDFYVSPTFQDLLNRTTAGQP | fused to the N- |
| | TGWYKDLRHYYYRARWELYDRSRDPHETQNLATDPRFAQLLEMLRDQLAK | terminus of an Fc |
| | WQWETHDPWVCAPDGVLEEKLSPQCQPLHNELGGGGSGGGGSDKTHTCPP | sequence with |
| | CPAPEAAGGPSVFLFPPKPKDTLMISRTPEVTCVVVDVSHEDPEVKFNWY | hole and LALA |
| | VDGVEVHNAKTKPREEQYNSTYRVVSVLTVLHQDWLNGKEYKCKVSNKAL | mutations |
| | PAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSCSVM | |
| | HEALHNHYTQKSLSLSPG | |
| 83 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSGGGGSDKTHTC | (G₄S)₂ linker |
| | PPCPAPEAAGGPSVFLFPPKPKDTLMISRTPEVTCVVVDVSHEDPEVKFN | (SEQ ID NO: 9) |
| | WYVDGVEVHNAKTKPREEQYNSTYRVVSVLTVLHQDWLNGKEYKCKVSNK | fused to the N- |
| | ALPAPIEKTISKAKGQPREPQVYTLPPSRDELTKNQVSLSCAVKGFYPSD | terminus of an Fc |
| | IAVEWESNGQPENNYKTTPPVLDSDGSFFLVSKLTVDKSRWQQGNVFSCS | sequence with |
| | VMHEALHNHYTQKSLSLSPGK | hole and LALA |
| | | mutations |
| 84 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSGGGGSDKTHTC | (G₄S)₂ linker |
| | PPCPAPEAAGGPSVFLFPPKPKDTLMISRTPEVTCVVVDVSHEDPEVKFN | (SEQ ID NO: 9) |
| | WYVDGVEVHNAKTKPREEQYNSTYRVVSVLTVLHQDWLNGKEYKCKVSNK | fused to the N- |
| | ALPAPIEKTISKAKGQPREPQVYTLPPSRDELTKNQVSLSCAVKGFYPSD | terminus of an Fc |
| | IAVEWESNGQPENNYKTTPPVLDSDGSFFLVSKLTVDKSRWQQGNVFSCS | sequence with |
| | VMHEALHNHYTQKSLSLSPG | hole and LALA |
| | | mutations |
| 85 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | (G₄S)₂ linker |
| | TILDWFSIPYPSYAIFGSKTIHLTGRSLLPALEAEPLWATVFGSQSHHEV | (SEQ ID NO: 9) |
| | TMSYPMRSVQHRHFRLVHNLNFKMPFPIDQDFYVSPTFQDLLNRTTAGQP | fused to the N- |
| | TGWYKDLRHYYYRARWELYDRSRDPHETQNLATDPRFAQLLEMLRDQLAK | terminus of an Fc |
| | WQWETHDPWVCAPDGVLEEKLSPQCQPLHNELGGGGSGGGGSDKTHTCPP | sequence with |
| | CPAPEAAGGPSVFLFPPKPKDTLMISRTPEVTCVVVDVSHEDPEVKFNWY | knob and LALA |
| | VDGVEVHNAKTKPREEQYNSTYRVVSVLTVLHQDWLNGKEYKCKVSNKAL | mutations |
| | PAPIEKTISKAKGQPREPQVYTLPPSRDELTKNQVSLWCLVKGFYPSDIA | |
| | VEWESNGQPENNYKTTPPVLDSDGSFFLYSKLTVDKSRWQQGNVFSCSVM | |
| | HEALHNHYTQKSLSLSPGK | |
| 86 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | (G₄S)₂ linker |
| | TILDWFSIPYPSYAIFGSKTIHLTGRSLLPALEAEPLWATVFGSQSHHEV | (SEQ ID NO: 9) |
| | TMSYPMRSVQHRHFRLVHNLNFKMPFPIDQDFYVSPTFQDLLNRTTAGQP | fused to the N- |
| | TGWYKDLRHYYYRARWELYDRSRDPHETQNLATDPRFAQLLEMLRDQLAK | terminus of an Fc |
| | WQWETHDPWVCAPDGVLEEKLSPQCQPLHNELGGGGSGGGGSDKTHTCPP | sequence with |
| | CPAPEAAGGPSVFLFPPKPKDTLMISRTPEVTCVVVDVSHEDPEVKFNWY | knob and LALA |
| | VDGVEVHNAKTKPREEQYNSTYRVVSVLTVLHQDWLNGKEYKCKVSNKAL | mutations |
| | PAPIEKTISKAKGQPREPQVYTLPPSRDELTKNQVSLWCLVKGFYPSDIA | |
| | VEWESNGQPENNYKTTPPVLDSDGSFFLYSKLTVDKSRWQQGNVFSCSVM | |
| | HEALHNHYTQKSLSLSPG | |
| 87 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSGGGGSDKTHTC | (G₄S)₂ linker |
| | PPCPAPEAAGGPSVFLFPPKPKDTLMISRTPEVTCVVVDVSHEDPEVKFN | (SEQ ID NO: 9) |
| | WYVDGVEVHNAKTKPREEQYNSTYRVVSVLTVLHQDWLNGKEYKCKVSNK | fused to the N- |
| | ALPAPIEKTISKAKGQPREPQVYTLPPSRDELTKNQVSLWCLVKGFYPSD | terminus of an Fc |
| | IAVEWESNGQPENNYKTTPPVLDSDGSFFLYSKLTVDKSRWQQGNVFSCS | sequence with |
| | VMHEALHNHYTQKSLSLSPGK | knob and LALA |
| | | mutations |
| 88 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSGGGGSDKTHTC | (G₄S)₂ linker |
| | PPCPAPEAAGGPSVFLFPPKPKDTLMISRTPEVTCVVVDVSHEDPEVKFN | (SEQ ID NO: 9) |
| | WYVDGVEVHNAKTKPREEQYNSTYRVVSVLTVLHQDWLNGKEYKCKVSNK | fused to the N- |
| | ALPAPIEKTISKAKGQPREPQVYTLPPSRDELTKNQVSLWCLVKGFYPSD | terminus of an Fc |
| | IAVEWESNGQPENNYKTTPPVLDSDGSFFLYSKLTVDKSRWQQGNVFSCS | sequence with |
| | VMHEALHNHYTQKSLSLSPG | knob and LALA |
| | | mutations |
| 89 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | G₄S linker (SEQ |
| | TILDWFSIPYPSYAIFGSKTIHLTGRSLLPALEAEPLWATVFGSQSHHEV | ID NO: 8) fused |
| | TMSYPMRSVQHRHFRLVHNLNFKMPFPIDQDFYVSPTFQDLLNRTTAGQP | to the N-terminus |
| | TGWYKDLRHYYYRARWELYDRSRDPHETQNLATDPRFAQLLEMLRDQLAK | of clone |
| | WQWETHDPWVCAPDGVLEEKLSPQCQPLHNELGGGGSDKTHTCPPCPAPE | CH3C.35.23.2 with |
| | AAGGPSVFLFPPKPKDTLMISRTPEVTCVVVDVSHEDPEVKFNWYVDGVE | knob and LALA |
| | VHNAKTKPREEQYNSTYRVVSVLTVLHQDWLNGKEYKCKVSNKALPAPIE | mutations |
| | KTISKAKGQPREPQVYTLPPSRDELTKNQVSLWCLVKGFYPSDIAVEWES | |
| | YGTEWANYKTTPPVLDSDGSFFLYSKLTVTKEEWQQGFVFSCSVMHEALH | |
| | NHYTQKSLSLSPGK | |
| 90 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | G₄S linker (SEQ |
| | TILDWFSIPYPSYAIFGSKTIHLTGRSLLPALEAEPLWATVFGSQSHHEV | ID NO: 8) fused |
| | TMSYPMRSVQHRHFRLVHNLNFKMPFPIDQDFYVSPTFQDLLNRTTAGQP | to the N-terminus |
| | TGWYKDLRHYYYRARWELYDRSRDPHETQNLATDPRFAQLLEMLRDQLAK | of clone |
| | WQWETHDPWVCAPDGVLEEKLSPQCQPLHNELGGGGSDKTHTCPPCPAPE | CH3C.35.23.2 with |
| | AAGGPSVFLFPPKPKDTLMISRTPEVTCVVVDVSHEDPEVKFNWYVDGVE | knob and LALA |
| | VHNAKTKPREEQYNSTYRVVSVLTVLHQDWLNGKEYKCKVSNKALPAPIE | mutations |
| | KTISKAKGQPREPQVYTLPPSRDELTKNQVSLWCLVKGFYPSDIAVEWES | |
| | YGTEWANYKTTPPVLDSDGSFFLYSKLTVTKEEWQQGFVFSCSVMHEALH | |
| | NHYTQKSLSLSPG | |
| 91 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSDKTHTCPPCPA | G₄S linker (SEQ |
| | PEAAGGPSVFLFPPKPKDTLMISRTPEVTCVVVDVSHEDPEVKFNWYVDG | ID NO: 8) fused |
| | VEVHNAKTKPREEQYNSTYRVVSVLTVLHQDWLNGKEYKCKVSNKALPAP | to the N-terminus |
| | IEKTISKAKGQPREPQVYTLPPSRDELTKNQVSLWCLVKGFYPSDIAVEW | of clone |
| | ESYGTEWANYKTTPPVLDSDGSFFLYSKLTVTKEEWQQGFVFSCSVMHEA | CH3C.35.23.2 with |
| | LHNHYTQKSLSLSPGK | knob and LALA |
| | | mutations |
| 92 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSDKTHTCPPCPA | G₄S linker (SEQ |
| | PEAAGGPSVFLFPPKPKDTLMISRTPEVTCVVVDVSHEDPEVKFNWYVDG | ID NO: 8) fused |
| | VEVHNAKTKPREEQYNSTYRVVSVLTVLHQDWLNGKEYKCKVSNKALPAP | to the N-terminus |
| | IEKTISKAKGQPREPQVYTLPPSRDELTKNQVSLWCLVKGFYPSDIAVEW | of clone |
| | ESYGTEWANYKTTPPVLDSDGSFFLYSKLTVTKEEWQQGFVFSCSVMHEA | CH3C.35.23.2 with |
| | LHNHYTQKSLSLSPG | knob and LALA |
| | | mutations |
| 93 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | G₄S linker (SEQ |
| | TILDWFSIPYPSYAIFGSKTIHLTGRSLLPALEAEPLWATVFGSQSHHEV | ID NO: 8) fused |
| | TMSYPMRSVQHRHFRLVHNLNFKMPFPIDQDFYVSPTFQDLLNRTTAGQP | to the N-terminus |
| | TGWYKDLRHYYYRARWELYDRSRDPHETQNLATDPRFAQLLEMLRDQLAK | of clone |
| | WQWETHDPWVCAPDGVLEEKLSPQCQPLHNELGGGGSDKTHTCPPCPAPE | CH3C.35.23.2 with |
| | AAGGPSVFLFPPKPKDTLMISRTPEVTCVVVDVSHEDPEVKFNWYVDGVE | knob and LALAPS |
| | VHNAKTKPREEQYNSTYRVVSVLTVLHQDWLNGKEYKCKVSNKALSAPIE | mutations |
| | KTISKAKGQPREPQVYTLPPSRDELTKNQVSLWCLVKGFYPSDIAVEWES | |
| | YGTEWANYKTTPPVLDSDGSFFLYSKLTVTKEEWQQGFVFSCSVMHEALH | |
| | NHYTQKSLSLSPGK | |
| 94 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | G₄S linker (SEQ |
| | TILDWFSIPYPSYAIFGSKTIHLTGRSLLPALEAEPLWATVFGSQSHHEV | ID NO: 8) fused |
| | TMSYPMRSVQHRHFRLVHNLNFKMPFPIDQDFYVSPTFQDLLNRTTAGQP | to the N-terminus |
| | TGWYKDLRHYYYRARWELYDRSRDPHETQNLATDPRFAQLLEMLRDQLAK | of clone |
| | WQWETHDPWVCAPDGVLEEKLSPQCQPLHNELGGGGSDKTHTCPPCPAPE | CH3C.35.23.2 with |
| | AAGGPSVFLFPPKPKDTLMISRTPEVTCVVVDVSHEDPEVKFNWYVDGVE | knob and LALAPS |
| | VHNAKTKPREEQYNSTYRVVSVLTVLHQDWLNGKEYKCKVSNKALSAPIE | mutations |
| | KTISKAKGQPREPQVYTLPPSRDELTKNQVSLWCLVKGFYPSDIAVEWES | |
| | YGTEWANYKTTPPVLDSDGSFFLYSKLTVTKEEWQQGFVFSCSVMHEALH | |
| | NHYTQKSLSLSPG | |
| 95 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSDKTHTCPPCPA | G₄S linker (SEQ |
| | PEAAGGPSVFLFPPKPKDTLMISRTPEVTCVVVDVSHEDPEVKFNWYVDG | ID NO: 8) fused |
| | VEVHNAKTKPREEQYNSTYRVVSVLTVLHQDWLNGKEYKCKVSNKALSAP | to the N-terminus |
| | IEKTISKAKGQPREPQVYTLPPSRDELTKNQVSLWCLVKGFYPSDIAVEW | of clone |
| | ESYGTEWANYKTTPPVLDSDGSFFLYSKLTVTKEEWQQGFVFSCSVMHEA | CH3C.35.23.2 with |
| | LHNHYTQKSLSLSPGK | knob and LALAPS |
| | | mutations |
| 96 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSDKTHTCPPCPA | G₄S linker (SEQ |
| | PEAAGGPSVFLFPPKPKDTLMISRTPEVTCVVVDVSHEDPEVKFNWYVDG | ID NO: 8) fused |
| | VEVHNAKTKPREEQYNSTYRVVSVLTVLHQDWLNGKEYKCKVSNKALSAP | to the N-terminus |
| | IEKTISKAKGQPREPQVYTLPPSRDELTKNQVSLWCLVKGFYPSDIAVEW | of clone |
| | ESYGTEWANYKTTPPVLDSDGSFFLYSKLTVTKEEWQQGFVFSCSVMHEA | CH3C.35.23.2 with |
| | LHNHYTQKSLSLSPG | knob and LALAPS |
| | | mutations |
| 97 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | G₄S linker (SEQ |
| | TILDWFSIPYPSYAIFGSKTIHLTGRSLLPALEAEPLWATVFGSQSHHEV | ID NO: 8) fused |
| | TMSYPMRSVQHRHFRLVHNLNFKMPFPIDQDFYVSPTFQDLLNRTTAGQP | to the N-terminus |
| | TGWYKDLRHYYYRARWELYDRSRDPHETQNLATDPRFAQLLEMLRDQLAK | of clone |
| | WQWETHDPWVCAPDGVLEEKLSPQCQPLHNELGGGGSDKTHTCPPCPAPE | CH3C.35.23.2 with |
| | AAGGPSVFLFPPKPKDTLMISRTPEVTCVVVDVSHEDPEVKFNWYVDGVE | hole and LALA |
| | VHNAKTKPREEQYNSTYRVVSVLTVLHQDWLNGKEYKCKVSNKALPAPIE | mutations |
| | KTISKAKGQPREPQVYTLPPSRDELTKNQVSLSCAVKGFYPSDIAVEWES | |
| | YGTEWANYKTTPPVLDSDGSFFLVSKLTVTKEEWQQGFVFSCSVMHEALH | |
| | NHYTQKSLSLSPGK | |
| 98 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | G₄S linker (SEQ |
| | TILDWFSIPYPSYAIFGSKTIHLTGRSLLPALEAEPLWATVFGSQSHHEV | ID NO: 8) fused |
| | TMSYPMRSVQHRHFRLVHNLNFKMPFPIDQDFYVSPTFQDLLNRTTAGQP | to the N-terminus |
| | TGWYKDLRHYYYRARWELYDRSRDPHETQNLATDPRFAQLLEMLRDQLAK | of clone |
| | WQWETHDPWVCAPDGVLEEKLSPQCQPLHNELGGGGSDKTHTCPPCPAPE | CH3C.35.23.2 with |
| | AAGGPSVFLFPPKPKDTLMISRTPEVTCVVVDVSHEDPEVKFNWYVDGVE | hole and LALA |
| | VHNAKTKPREEQYNSTYRVVSVLTVLHQDWLNGKEYKCKVSNKALPAPIE | mutations |
| | KTISKAKGQPREPQVYTLPPSRDELTKNQVSLSCAVKGFYPSDIAVEWES | |
| | YGTEWANYKTTPPVLDSDGSFFLVSKLTVTKEEWQQGFVFSCSVMHEALH | |
| | NHYTQKSLSLSPG | |
| 99 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSDKTHTCPPCPA | G₄S linker (SEQ |
| | PEAAGGPSVFLFPPKPKDTLMISRTPEVTCVVVDVSHEDPEVKFNWYVDG | ID NO: 8) fused |
| | VEVHNAKTKPREEQYNSTYRVVSVLTVLHQDWLNGKEYKCKVSNKALPAP | to the N-terminus |
| | IEKTISKAKGQPREPQVYTLPPSRDELTKNQVSLSCAVKGFYPSDIAVEW | of clone |
| | ESYGTEWANYKTTPPVLDSDGSFFLVSKLTVTKEEWQQGFVFSCSVMHEA | CH3C.35.23.2 with |
| | LHNHYTQKSLSLSPGK | hole and LALA |
| | | mutations |
| 100 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSDKTHTCPPCPA | G₄S linker (SEQ |
| | PEAAGGPSVFLFPPKPKDTLMISRTPEVTCVVVDVSHEDPEVKFNWYVDG | ID NO: 8) fused |
| | VEVHNAKTKPREEQYNSTYRVVSVLTVLHQDWLNGKEYKCKVSNKALPAP | to the N-terminus |
| | IEKTISKAKGQPREPQVYTLPPSRDELTKNQVSLSCAVKGFYPSDIAVEW | of clone |
| | ESYGTEWANYKTTPPVLDSDGSFFLVSKLTVTKEEWQQGFVFSCSVMHEA | CH3C.35.23.2 with |
| | LHNHYTQKSLSLSPG | hole and LALA |
| | | mutations |
| 101 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | GS linker |
| | TILDWFSIPYPSYAIFGSKTIHLTGRSLLPALEAEPLWATVFGSQSHHEV | fused to |
| | TMSYPMRSVQHRHFRLVHNLNFKMPFPIDQDFYVSPTFQDLLNRTTAGQP | the N-terminus of |
| | TGWYKDLRHYYYRARWELYDRSRDPHETQNLATDPRFAQLLEMLRDQLAK | clone |
| | WQWETHDPWVCAPDGVLEEKLSPQCQPLHNELGSDKTHTCPPCPAPEAAG | CH3C.35.23.2 with |
| | GPSVFLFPPKPKDTLMISRTPEVTCVVVDVSHEDPEVKFNWYVDGVEVHN | knob and LALA |
| | AKTKPREEQYNSTYRVVSVLTVLHQDWLNGKEYKCKVSNKALPAPIEKTI | mutations |
| | SKAKGQPREPQVYTLPPSRDELTKNQVSLWCLVKGFYPSDIAVEWESYGT | |
| | EWANYKTTPPVLDSDGSFFLYSKLTVTKEEWQQGFVFSCSVMHEALHNHY | |
| | TQKSLSLSPG | |
| 102 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | GS linker |
| | TILDWFSIPYPSYAIFGSKTIHLTGRSLLPALEAEPLWATVFGSQSHHEV | fused to |
| | TMSYPMRSVQHRHFRLVHNLNFKMPFPIDQDFYVSPTFQDLLNRTTAGQP | the N-terminus of |
| | TGWYKDLRHYYYRARWELYDRSRDPHETQNLATDPRFAQLLEMLRDQLAK | clone |
| | WQWETHDPWVCAPDGVLEEKLSPQCQPLHNELGSDKTHTCPPCPAPEAAG | CH3C.35.23.2 with |
| | GPSVFLFPPKPKDTLMISRTPEVTCVVVDVSHEDPEVKFNWYVDGVEVHN | knob and LALA |
| | AKTKPREEQYNSTYRVVSVLTVLHQDWLNGKEYKCKVSNKALPAPIEKTI | mutations |
| | SKAKGQPREPQVYTLPPSRDELTKNQVSLWCLVKGFYPSDIAVEWESYGT | |
| | EWANYKTTPPVLDSDGSFFLYSKLTVTKEEWQQGFVFSCSVMHEALHNHY | |
| | TQKSLSLSPG | |
| 103 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGSDKTHTCPPCPAPEA | GS linker |
| | AGGPSVFLFPPKPKDTLMISRTPEVTCVVVDVSHEDPEVKFNWYVDGVEV | fused to |
| | HNAKTKPREEQYNSTYRVVSVLTVLHQDWLNGKEYKCKVSNKALPAPIEK | the N-terminus of |
| | TISKAKGQPREPQVYTLPPSRDELTKNQVSLWCLVKGFYPSDIAVEWESY | clone |
| | GTEWANYKTTPPVLDSDGSFFLYSKLTVTKEEWQQGFVFSCSVMHEALHN | CH3C.35.23.2 with |
| | HYTQKSLSLSPGK | knob and LALA |
| | | mutations |
| 104 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGSDKTHTCPPCPAPEA | GS linker |
| | AGGPSVFLFPPKPKDTLMISRTPEVTCVVVDVSHEDPEVKFNWYVDGVEV | fused to |
| | HNAKTKPREEQYNSTYRVVSVLTVLHQDWLNGKEYKCKVSNKALPAPIEK | the N-terminus of |
| | TISKAKGQPREPQVYTLPPSRDELTKNQVSLWCLVKGFYPSDIAVEWESY | clone |
| | GTEWANYKTTPPVLDSDGSFFLYSKLTVTKEEWQQGFVFSCSVMHEALHN | CH3C.35.23.2 with |
| | HYTQKSLSLSPG | knob and LALA |
| | | mutations |
| 105 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | GS linker |
| | TILDWFSIPYPSYAIFGSKTIHLTGRSLLPALEAEPLWATVFGSQSHHEV | fused to |
| | TMSYPMRSVQHRHFRLVHNLNFKMPFPIDQDFYVSPTFQDLLNRTTAGQP | the N-terminus of |
| | TGWYKDLRHYYYRARWELYDRSRDPHETQNLATDPRFAQLLEMLRDQLAK | clone |
| | WQWETHDPWVCAPDGVLEEKLSPQCQPLHNELGSDKTHTCPPCPAPEAAG | CH3C.35.23.2 with |
| | GPSVFLFPPKPKDTLMISRTPEVTCVVVDVSHEDPEVKFNWYVDGVEVHN | hole and LALA |
| | AKTKPREEQYNSTYRVVSVLTVLHQDWLNGKEYKCKVSNKALPAPIEKTI | mutations |
| | SKAKGQPREPQVYTLPPSRDELTKNQVSLSCAVKGFYPSDIAVEWESYGT | |
| | EWANYKTTPPVLDSDGSFFLVSKLTVTKEEWQQGFVFSCSVMHEALHNHY | |
| | TQKSLSLSPGK | |
| 106 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | GS linker |
| | TILDWFSIPYPSYAIFGSKTIHLTGRSLLPALEAEPLWATVFGSQSHHEV | fused to |
| | TMSYPMRSVQHRHFRLVHNLNFKMPFPIDQDFYVSPTFQDLLNRTTAGQP | the N-terminus of |
| | TGWYKDLRHYYYRARWELYDRSRDPHETQNLATDPRFAQLLEMLRDQLAK | clone |
| | WQWETHDPWVCAPDGVLEEKLSPQCQPLHNELGSDKTHTCPPCPAPEAAG | CH3C.35.23.2 with |
| | GPSVFLFPPKPKDTLMISRTPEVTCVVVDVSHEDPEVKFNWYVDGVEVHN | hole and LALA |
| | AKTKPREEQYNSTYRVVSVLTVLHQDWLNGKEYKCKVSNKALPAPIEKTI | mutations |
| | SKAKGQPREPQVYTLPPSRDELTKNQVSLSCAVKGFYPSDIAVEWESYGT | |
| | EWANYKTTPPVLDSDGSFFLVSKLTVTKEEWQQGFVFSCSVMHEALHNHY | |
| | TQKSLSLSPG | |
| 107 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGSDKTHTCPPCPAPEA | GS linker |
| | AGGPSVFLFPPKPKDTLMISRTPEVTCVVVDVSHEDPEVKFNWYVDGVEV | fused to |
| | HNAKTKPREEQYNSTYRVVSVLTVLHQDWLNGKEYKCKVSNKALPAPIEK | the N-terminus of |
| | TISKAKGQPREPQVYTLPPSRDELTKNQVSLSCAVKGFYPSDIAVEWESY | clone |
| | GTEWANYKTTPPVLDSDGSFFLVSKLTVTKEEWQQGFVFSCSVMHEALHN | CH3C.35.23.2 with |
| | HYTQKSLSLSPGK | hole and LALA |
| | | mutations |
| 108 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGSDKTHTCPPCPAPEA | GS linker |
| | AGGPSVFLFPPKPKDTLMISRTPEVTCVVVDVSHEDPEVKFNWYVDGVEV | fused to |
| | HNAKTKPREEQYNSTYRVVSVLTVLHQDWLNGKEYKCKVSNKALPAPIEK | the N-terminus of |
| | TISKAKGQPREPQVYTLPPSRDELTKNQVSLSCAVKGFYPSDIAVEWESY | clone |
| | GTEWANYKTTPPVLDSDGSFFLVSKLTVTKEEWQQGFVFSCSVMHEALHN | CH3C.35.23.2 with |
| | HYTQKSLSLSPG | hole and LALA |
| | | mutations |
| 109 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | (G₄S)₂ linker |
| | TILDWFSIPYPSYAIFGSKTIHLTGRSLLPALEAEPLWATVFGSQSHHEV | (SEQ ID NO: 9) |
| | TMSYPMRSVQHRHFRLVHNLNFKMPFPIDQDFYVSPTFQDLLNRTTAGQP | fused to the N- |
| | TGWYKDLRHYYYRARWELYDRSRDPHETQNLATDPRFAQLLEMLRDQLAK | terminus of clone |
| | WQWETHDPWVCAPDGVLEEKLSPQCQPLHNELGGGGSGGGGSDKTHTCPP | CH3C.35.23.2 with |
| | CPAPEAAGGPSVFLFPPKPKDTLMISRTPEVTCVVVDVSHEDPEVKFNWY | knob and LALA |
| | VDGVEVHNAKTKPREEQYNSTYRVVSVLTVLHQDWLNGKEYKCKVSNKAL | mutations |
| | PAPIEKTISKAKGQPREPQVYTLPPSRDELTKNQVSLWCLVKGFYPSDIA | |
| | VEWESYGTEWANYKTTPPVLDSDGSFFLYSKLTVTKEEWQQGFVFSCSVM | |
| | HEALHNHYTQKSLSLSPGK | |
| 110 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | mutations |
| | IGKKHVGPETVYPFDFAYTEENGSVLQVGRNITRIKLLVRKFLQTQDDRP | polypeptide with |
| | FFLYVAFHDPHRCGHSQPQYGTFCEKFGNGESGMGRIPDWTPQAYDPLDV | mature human SGSH |
| | LVPYFVPNTPAARADLAAQYTTVGRMDQGVGLVLQELRDAGVLNDTLVIF | sequence |
| | TSDNGIPFPSGRTNLYWPGTAEPLLVSSPEHPKRWGQVSEAYVSLLDLTP | (underlined) with |
| | TILDWFSIPYPSYAIFGSKTIHLTGRSLLPALEAEPLWATVFGSQSHHEV | (G₄S)₂ linker |
| | TMSYPMRSVQHRHFRLVHNLNFKMPFPIDQDFYVSPTFQDLLNRTTAGQP | (SEQ ID NO: 9) |
| | TGWYKDLRHYYYRARWELYDRSRDPHETQNLATDPRFAQLLEMLRDQLAK | fused to the N- |
| | WQWETHDPWVCAPDGVLEEKLSPQCQPLHNELGGGGSGGGGSDKTHTCPP | terminus of clone |
| | CPAPEAAGGPSVFLFPPKPKDTLMISRTPEVTCVVVDVSHEDPEVKFNWY | CH3C.35.23.2 with |
| | VDGVEVHNAKTKPREEQYNSTYRVVSVLTVLHQDWLNGKEYKCKVSNKAL | knob and LALA |
| | PAPIEKTISKAKGQPREPQVYTLPPSRDELTKNQVSLWCLVKGFYPSDIA | |
| | VEWESYGTEWANYKTTPPVLDSDGSFFLYSKLTVTKEEWQQGFVFSCSVM | |
| | HEALHNHYTQKSLSLSPG | |
| 111 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSGGGGSDKTHTC | (G₄S)₂ linker |
| | PPCPAPEAAGGPSVFLFPPKPKDTLMISRTPEVTCVVVDVSHEDPEVKFN | (SEQ ID NO: 9) |
| | WYVDGVEVHNAKTKPREEQYNSTYRVVSVLTVLHQDWLNGKEYKCKVSNK | fused to the N- |
| | ALPAPIEKTISKAKGQPREPQVYTLPPSRDELTKNQVSLWCLVKGFYPSD | terminus of clone |
| | IAVEWESYGTEWANYKTTPPVLDSDGSFFLYSKLTVTKEEWQQGFVFSCS | CH3C.35.23.2 with |
| | VMHEALHNHYTQKSLSLSPGK | knob and LALA |
| | | mutations |
| 112 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSGGGGSDKTHTC | (G₄S)₂ linker |
| | PPCPAPEAAGGPSVFLFPPKPKDTLMISRTPEVTCVVVDVSHEDPEVKFN | (SEQ ID NO: 9) |
| | WYVDGVEVHNAKTKPREEQYNSTYRVVSVLTVLHQDWLNGKEYKCKVSNK | fused to the N- |
| | ALPAPIEKTISKAKGQPREPQVYTLPPSRDELTKNQVSLWCLVKGFYPSD | terminus of clone |
| | IAVEWESYGTEWANYKTTPPVLDSDGSFFLYSKLTVTKEEWQQGFVFSCS | CH3C.35.23.2 with |
| | VMHEALHNHYTQKSLSLSPG | knob and LALA |
| | | mutations |
| 113 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | (G₄S)₂ linker |
| | TILDWFSIPYPSYAIFGSKTIHLTGRSLLPALEAEPLWATVFGSQSHHEV | (SEQ ID NO: 9) |
| | TMSYPMRSVQHRHFRLVHNLNFKMPFPIDQDFYVSPTFQDLLNRTTAGQP | fused to the N- |
| | TGWYKDLRHYYYRARWELYDRSRDPHETQNLATDPRFAQLLEMLRDQLAK | terminus of clone |
| | WQWETHDPWVCAPDGVLEEKLSPQCQPLHNELGGGGSGGGGSDKTHTCPP | CH3C.35.23.2 with |
| | CPAPEAAGGPSVFLFPPKPKDTLMISRTPEVTCVVVDVSHEDPEVKFNWY | hole and LALA |
| | VDGVEVHNAKTKPREEQYNSTYRVVSVLTVLHQDWLNGKEYKCKVSNKAL | mutations |
| | PAPIEKTISKAKGQPREPQVYTLPPSRDELTKNQVSLSCAVKGFYPSDIA | |
| | VEWESYGTEWANYKTTPPVLDSDGSFFLVSKLTVTKEEWQQGFVFSCSVM | |
| | HEALHNHYTQKSLSLSPGK | |
| 114 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | (G₄S)₂ linker |
| | TILDWFSIPYPSYAIFGSKTIHLTGRSLLPALEAEPLWATVFGSQSHHEV | (SEQ ID NO: 9) |
| | TMSYPMRSVQHRHFRLVHNLNFKMPFPIDQDFYVSPTFQDLLNRTTAGQP | fused to the N- |
| | TGWYKDLRHYYYRARWELYDRSRDPHETQNLATDPRFAQLLEMLRDQLAK | terminus of clone |
| | WQWETHDPWVCAPDGVLEEKLSPQCQPLHNELGGGGSGGGGSDKTHTCPP | CH3C.35.23.2 with |
| | CPAPEAAGGPSVFLFPPKPKDTLMISRTPEVTCVVVDVSHEDPEVKFNWY | hole and LALA |
| | VDGVEVHNAKTKPREEQYNSTYRVVSVLTVLHQDWLNGKEYKCKVSNKAL | mutations |
| | PAPIEKTISKAKGQPREPQVYTLPPSRDELTKNQVSLSCAVKGFYPSDIA | |
| | VEWESYGTEWANYKTTPPVLDSDGSFFLVSKLTVTKEEWQQGFVFSCSVM | |
| | HEALHNHYTQKSLSLSPG | |
| 115 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSGGGGSDKTHTC | (G₄S)₂ linker |
| | PPCPAPEAAGGPSVFLFPPKPKDTLMISRTPEVTCVVVDVSHEDPEVKFN | (SEQ ID NO: 9) |
| | WYVDGVEVHNAKTKPREEQYNSTYRVVSVLTVLHQDWLNGKEYKCKVSNK | fused to the N- |
| | ALPAPIEKTISKAKGQPREPQVYTLPPSRDELTKNQVSLSCAVKGFYPSD | terminus of clone |
| | IAVEWESYGTEWANYKTTPPVLDSDGSFFLVSKLTVTKEEWQQGFVFSCS | CH3C.35.23.2 with |
| | VMHEALHNHYTQKSLSLSPGK | hole and LALA |
| | | mutations |
| 116 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSGGGGSDKTHTC | (G₄S)₂ linker |
| | PPCPAPEAAGGPSVFLFPPKPKDTLMISRTPEVTCVVVDVSHEDPEVKFN | (SEQ ID NO: 9) |
| | WYVDGVEVHNAKTKPREEQYNSTYRVVSVLTVLHQDWLNGKEYKCKVSNK | fused to the N- |
| | ALPAPIEKTISKAKGQPREPQVYTLPPSRDELTKNQVSLSCAVKGFYPSD | terminus of clone |
| | IAVEWESYGTEWANYKTTPPVLDSDGSFFLVSKLTVTKEEWQQGFVFSCS | CH3C.35.23.2 with |
| | VMHEALHNHYTQKSLSLSPG | hole and LALA |
| | | mutations |
| 117 | DKTHTCPPCPAPEAAGGPSVFLFPPKPKDTLMISRTPEVTCVVVDVSHED | Fc-SGSH fusion |
| | PEVKFNWYVDGVEVHNAKTKPREEQYNSTYRVVSVLTVLHQDWLNGKEYK | polypeptide with |
| | CKVSNKALPAPIEKTISKAKGQPREPQVYTLPPSRDELTKNQVSLSCAVK | mature human SGSH |
| | GFYPSDIAVEWESNGQPENNYKTTPPVLDSDGSFFLVSKLTVDKSRWQQG | sequence |
| | NVFSCSVMHEALHNHYTQKSLSLSPGGGGGSRPRNALLLLADDGGFESGA | (underlined) |
| | YNNSAIATPHLDALARRSLLFRNAFTSVSSCSPSRASLLTGLPQHQNGMY | fused to the C- |
| | GLHQDVHHFNSFDKVRSLPLLLSQAGVRTGIIGKKHVGPETVYPFDFAYT | terminus of an Fc |
| | EENGSVLQVGRNITRIKLLVRKFLQTQDDRPFFLYVAFHDPHRCGHSQPQ | sequence with |
| | YGTFCEKFGNGESGMGRIPDWTPQAYDPLDVLVPYFVPNTPAARADLAAQ | hole and LALA |
| | YTTVGRMDQGVGLVLQELRDAGVLNDTLVIFTSDNGIPFPSGRTNLYWPG | mutations |
| | TAEPLLVSSPEHPKRWGQVSEAYVSLLDLTPTILDWFSIPYPSYAIFGSK | |
| | TIHLTGRSLLPALEAEPLWATVFGSQSHHEVTMSYPMRSVQHRHFRLVHN | |
| | LNFKMPFPIDQDFYVSPTFQDLLNRTTAGQPTGWYKDLRHYYYRARWELY | |
| | DRSRDPHETQNLATDPRFAQLLEMLRDQLAKWQWETHDPWVCAPDGVLEE | |
| | KLSPQCQPLHNEL | |
| 118 | DKTHTCPPCPAPEAAGGPSVFLFPPKPKDTLMISRTPEVTCVVVDVSHED | Fc-SGSH fusion |
| | PEVKFNWYVDGVEVHNAKTKPREEQYNSTYRVVSVLTVLHQDWLNGKEYK | polypeptide with |
| | CKVSNKALPAPIEKTISKAKGQPREPQVYTLPPSRDELTKNQVSLSCAVK | mature human SGSH |
| | GFYPSDIAVEWESNGQPENNYKTTPPVLDSDGSFFLVSKLTVDKSRWQQG | sequence |
| | NVFSCSVMHEALHNHYTQKSLSLSPGGGGGSRPRNALLLLADDGGFESGA | (underlined; |
| | YNNSAIATPHLDALARRSLLFRNAFTSVSSfGSPSRASLLTGLPQHQNGM | formylglycine |
| | YGLHQDVHHFNSFDKVRSLPLLLSQAGVRTGIIGKKHVGPETVYPFDFAY | residue “fG” |
| | TEENGSVLQVGRNITRIKLLVRKFLQTQDDRPFFLYVAFHDPHRCGHSQP | double |
| | QYGTFCEKFGNGESGMGRIPDWTPQAYDPLDVLVPYFVPNTPAARADLAA | underlined) |
| | QYTTVGRMDQGVGLVLQELRDAGVLNDTLVIFTSDNGIPFPSGRTNLYWP | fused to the C- |
| | GTAEPLLVSSPEHPKRWGQVSEAYVSLLDLTPTILDWFSIPYPSYAIFGS | terminus of an Fc |
| | KTIHLTGRSLLPALEAEPLWATVFGSQSHHEVTMSYPMRSVQHRHFRLVH | sequence with |
| | NLNFKMPFPIDQDFYVSPTFQDLLNRTTAGQPTGWYKDLRHYYYRARWEL | hole and LALA |
| | YDRSRDPHETQNLATDPRFAQLLEMLRDQLAKWQWETHDPWVCAPDGVLE | mutations |
| | EKLSPQCQPLHNEL | |
| 119 | DKTHTCPPCPAPEAAGGPSVFLFPPKPKDTLMISRTPEVTCVVVDVSHED | Fc-SGSH fusion |
| | PEVKFNWYVDGVEVHNAKTKPREEQYNSTYRWSVLTVLHQDWLNGKEYK | polypeptide with |
| | CKVSNKALPAPIEKTISKAKGQPREPQVYTLPPSRDELTKNQVSLWCLVK | mature human SGSH |
| | GFYPSDIAVEWESYGTEWANYKTTPPVLDSDGSFFLYSKLTVTKEEWQQG | sequence |
| | FVFSCSVMHEALHNHYTQKSLSLSPGGGGGSRPRNALLLLADDGGFESGA | (underlined) |
| | YNNSAIATPHLDALARRSLLFRNAFTSVSSCSPSRASLLTGLPQHQNGMY | fused to the C- |
| | GLHQDVHHFNSFDKVRSLPLLLSQAGVRTGIIGKKHVGPETVYPFDFAYT | terminus of clone |
| | EENGSVLQVGRNITRIKLLVRKFLQTQDDRPFFLYVAFHDPHRCGHSQPQ | CH3C.35.23.2 with |
| | YGTFCEKFGNGESGMGRIPDWTPQAYDPLDVLVPYFVPNTPAARADLAAQ | knob and LALA |
| | YTTVGRMDQGVGLVLQELRDAGVLNDTLVIFTSDNGIPFPSGRTNLYWPG | mutations |
| | TAEPLLVSSPEHPKRWGQVSEAYVSLLDLTPTILDWFSIPYPSYAIFGSK | |
| | TIHLTGRSLLPALEAEPLWATVFGSQSHHEVTMSYPMRSVQHRHFRLVHN | |
| | LNFKMPFPIDQDFYVSPTFQDLLNRTTAGQPTGWYKDLRHYYYRARWELY | |
| | DRSRDPHETQNLATDPRFAQLLEMLRDQLAKWQWETHDPWVCAPDGVLEE | |
| | KLSPQCQPLHNEL | |
| 120 | DKTHTCPPCPAPEAAGGPSVFLFPPKPKDTLMISRTPEVTCVVVDVSHED | Fc-SGSH fusion |
| | PEVKFNWYVDGVEVHNAKTKPREEQYNSTYRVVSVLTVLHQDWLNGKEYK | polypeptide with |
| | CKVSNKALPAPIEKTISKAKGQPREPQVYTLPPSRDELTKNQVSLWCLVK | mature human SGSH |
| | GFYPSDIAVEWESYGTEWANYKTTPPVLDSDGSFFLYSKLTVTKEEWQQG | sequence |
| | FVFSCSVMHEALHNHYTQKSLSLSPGGGGGSRPRNALLLLADDGGFESGA | (underlined; |
| | YNNSAIATPHLDALARRSLLFRNAFTSVSSfGSPSRASLLTGLPQHQNGM | formylglycine |
| | YGLHQDVHHFNSFDKVRSLPLLLSQAGVRTGIIGKKHVGPETVYPFDFAY | residue “fG” |
| | TEENGSVLQVGRNITRIKLLVRKFLQTQDDRPFFLYVAFHDPHRCGHSQP | double |
| | QYGTFCEKFGNGESGMGRIPDWTPQAYDPLDVLVPYFVPNTPAARADLAA | underlined) fused |
| | QYTTVGRMDQGVGLVLQELRDAGVLNDTLVIFTSDNGIPFPSGRTNLYWP | to the C-terminus |
| | GTAEPLLVSSPEHPKRWGQVSEAYVSLLDLTPTILDWFSIPYPSYAIFGS | of clone |
| | KTIHLTGRSLLPALEAEPLWATVFGSQSHHEVTMSYPMRSVQHRHFRLVH | CH3C.35.23.2 with |
| | NLNFKMPFPIDQDFYVSPTFQDLLNRTTAGQPTGWYKDLRHYYYRARWEL | knob and LALA |
| | YDRSRDPHETQNLATDPRFAQLLEMLRDQLAKWQWETHDPWVCAPDGVLE | mutations |
| | EKLSPQCQPLHNEL | |
| 121 | MGWSCIILFLVATATGAYA | Secretion signal |
| | | peptide |
| 122 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | G₄S linker (SEQ |
| | TILDWFSIPYPSYAIFGSKTIHLTGRSLLPALEAEPLWATVFGSQSHHEV | ID NO: 8) fused |
| | TMSYPMRSVQHRHFRLVHNLNFKMPFPIDQDFYVSPTFQDLLNRTTAGQP | to the N-terminus |
| | TGWYKDLRHYYYRARWELYDRSRDPHETQNLATDPRFAQLLEMLRDQLAK | of clone |
| | WQWETHDPWVCAPDGVLEEKLSPQCQPLHNELGGGGSDKTHTCPPCPAPE | CH3C.35.21.17 |
| | AAGGPSVFLFPPKPKDTLMISRTPEVTCVVVDVSHEDPEVKFNWYVDGVE | with knob and |
| | VHNAKTKPREEQYNSTYRVVSVLTVLHQDWLNGKEYKCKVSNKALPAPIE | LALA mutations |
| | KTISKAKGQPREPQVYTLPPSRDELTKNQVSLWCLVKGFYPSDIAVLWES | |
| | YGTEWSSYKTTPPVLDSDGSFFLYSKLTVTKEEWQQGFVFSCSVMHEALH | |
| | NHYTQKSLSLSPGK | |
| 123 | RPRNALLLLADDGGFESGAYNNSAIATPHLDALARRSLLFRNAFTSVSSC | SGSH-Fc fusion |
| | SPSRASLLTGLPQHQNGMYGLHQDVHHFNSFDKVRSLPLLLSQAGVRTGI | polypeptide with |
| | IGKKHVGPETVYPFDFAYTEENGSVLQVGRNITRIKLLVRKFLQTQDDRP | mature human SGSH |
| | FFLYVAFHDPHRCGHSQPQYGTFCEKFGNGESGMGRIPDWTPQAYDPLDV | sequence |
| | LVPYFVPNTPAARADLAAQYTTVGRMDQGVGLVLQELRDAGVLNDTLVIF | (underlined) with |
| | TSDNGIPFPSGRTNLYWPGTAEPLLVSSPEHPKRWGQVSEAYVSLLDLTP | G₄S linker (SEQ |
| | TILDWFSIPYPSYAIFGSKTIHLTGRSLLPALEAEPLWATVFGSQSHHEV | ID NO: 8) fused |
| | TMSYPMRSVQHRHFRLVHNLNFKMPFPIDQDFYVSPTFQDLLNRTTAGQP | to the N-terminus |
| | TGWYKDLRHYYYRARWELYDRSRDPHETQNLATDPRFAQLLEMLRDQLAK | of clone |
| | WQWETHDPWVCAPDGVLEEKLSPQCQPLHNELGGGGSDKTHTCPPCPAPE | CH3C.35.21.17 |
| | AAGGPSVFLFPPKPKDTLMISRTPEVTCVVVDVSHEDPEVKFNWYVDGVE | with knob and |
| | VHNAKTKPREEQYNSTYRVVSVLTVLHQDWLNGKEYKCKVSNKALPAPIE | LALA mutations |
| | KTISKAKGQPREPQVYTLPPSRDELTKNQVSLWCLVKGFYPSDIAVLWES | |
| | YGTEWSSYKTTPPVLDSDGSFFLYSKLTVTKEEWQQGFVFSCSVMHEALH | |
| | NHYTQKSLSLSPG | |
| 124 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSDKTHTCPPCPA | G₄S linker (SEQ |
| | PEAAGGPSVFLFPPKPKDTLMISRTPEVTCVVVDVSHEDPEVKFNWYVDG | ID NO: 8) fused |
| | VEVHNAKTKPREEQYNSTYRVVSVLTVLHQDWLNGKEYKCKVSNKALPAP | to the N-terminus |
| | IEKTISKAKGQPREPQVYTLPPSRDELTKNQVSLWCLVKGFYPSDIAVLW | of clone |
| | ESYGTEWSSYKTTPPVLDSDGSFFLYSKLTVTKEEWQQGFVFSCSVMHEA | CH3C.35.21.17 |
| | LHNHYTQKSLSLSPGK | with knob and |
| | | LALA mutations |
| 125 | RPRNALLLLADDGGFESGAYNNSAIATPHLDALARRSLLFRNAFTSVSS | SGSH-Fc fusion |
| | fGSPSRASLLTGLPQHQNGMYGLHQDVHHFNSFDKVRSLPLLLSQAGVRT | polypeptide with |
| | GIIGKKHVGPETVYPFDFAYTEENGSVLQVGRNITRIKLLVRKFLQTQDD | mature human SGSH |
| | RPFFLYVAFHDPHRCGHSQPQYGTFCEKFGNGESGMGRIPDWTPQAYDPL | sequence |
| | DVLVPYFVPNTPAARADLAAQYTTVGRMDQGVGLVLQELRDAGVLNDTLV | (underlined; |
| | IFTSDNGIPFPSGRTNLYWPGTAEPLLVSSPEHPKRWGQVSEAYVSLLDL | formylglycine |
| | TPTILDWFSIPYPSYAIFGSKTIHLTGRSLLPALEAEPLWATVFGSQSHH | residue “fG” |
| | EVTMSYPMRSVQHRHFRLVHNLNFKMPFPIDQDFYVSPTFQDLLNRTTAG | double |
| | QPTGWYKDLRHYYYRARWELYDRSRDPHETQNLATDPRFAQLLEMLRDQL | underlined) with |
| | AKWQWETHDPWVCAPDGVLEEKLSPQCQPLHNELGGGGSDKTHTCPPCPA | G₄S linker (SEQ |
| | PEAAGGPSVFLFPPKPKDTLMISRTPEVTCVVVDVSHEDPEVKFNWYVDG | ID NO: 8) fused |
| | VEVHNAKTKPREEQYNSTYRVVSVLTVLHQDWLNGKEYKCKVSNKALPAP | to the N-terminus |
| | IEKTISKAKGQPREPQVYTLPPSRDELTKNQVSLWCLVKGFYPSDIAVLW | of clone |
| | ESYGTEWSSYKTTPPVLDSDGSFFLYSKLTVTKEEWQQGFVFSCSVMHEA | CH3C.35.21.17 |
| | LHNHYTQKSLSLSPG | with knob and |
| | | LALA mutations |
| 126 | CXPXR | CXPXR motif, |
| | | wherein “X” is |
| | | any amino acid |
| 127 | NAFTSVSSCSPSR | Tryptic peptide |
| | | embodiment |
| 128 | NAFTSVSSC(CAM)SPSR | Tryptic peptide |
| | | embodiment; |
| | | C(CAM) is |
| | | alkylated |
| | | carbamidomethyl |
| | | Cys |
| 129 | NAFTSVSS(Fgly)SPSR | Tryptic peptide |
| | | embodiment; Fgly |
| | | is formylglycine |

All publications, patents, and patent documents are incorporated by reference herein, as though individually incorporated by reference. The present disclosure has been described with reference to various specific and preferred embodiments and techniques. However, it should be understood that many variations and modifications may be made while remaining within the spirit and scope of the invention.

1-78. (canceled)

79. A method of treating Sanfilippo syndrome type A (MPS IIIA) in a patient in need thereof, the method comprising administering to the patient a protein comprising:
a. a first fusion polypeptide comprising a first Fc polypeptide linked to a first N-sulfoglucosamine sulfohydrolase (SGSH) amino acid sequence, wherein the first fusion polypeptide comprises any one of SEQ ID NOs: 65-68; and
b. a second fusion polypeptide comprising a second Fc polypeptide linked to a second SGSH amino acid sequence, wherein the second fusion polypeptide comprises any one of SEQ ID NOs: 93-96.

80-81. (canceled)

82. A method of decreasing the accumulation of a toxic metabolic product in a patient having Sanfilippo syndrome type A (MPS IIIA), the method comprising administering to the patient a protein comprising:
a. a first fusion polypeptide comprising a first Fc polypeptide linked to a first N-sulfoglucosamine sulfohydrolase (SGSH) amino acid sequence, wherein the first fusion polypeptide comprises any one of SEQ ID NOs: 65-68; and
b. a second fusion polypeptide comprising a second Fc polypeptide linked to a second SGSH amino acid sequence, wherein the second fusion polypeptide comprises any one of SEQ ID NOs: 93-96.

83-84. (canceled)

85. The method of claim 82, wherein the toxic metabolic product comprises heparan sulfate-derived oligosaccharides.

86. The method of claim 79, wherein the first fusion polypeptide comprises SEQ ID NO: 65.

87. The method of claim 79, wherein the first fusion polypeptide comprises SEQ ID NO: 66.

88. The method of claim 79, wherein the first fusion polypeptide comprises SEQ ID NO: 67.

89. The method of claim 79, wherein the first fusion polypeptide comprises SEQ ID NO: 68.

90. The method of claim 79, wherein the second fusion polypeptide comprises SEQ ID NO: 93.

91. The method of claim 79, wherein the second fusion polypeptide comprises SEQ ID NO: 94.

92. The method of claim 79, wherein the second fusion polypeptide comprises SEQ ID NO: 95.

93. The method of claim 79, wherein the second fusion polypeptide comprises SEQ ID NO: 96.

94. The method of claim 79, wherein the first fusion polypeptide comprises SEQ ID NO: 67 or 68; and the second fusion polypeptide comprises SEQ ID NO:95 or 96.

95. The method of claim 79, wherein the first fusion polypeptide is SEQ ID NO: 67 or 68; and the second fusion polypeptide is SEQ ID NO:95 or 96.

96. The method of claim 79, wherein the first fusion polypeptide comprises SEQ ID NO: 67; and the second fusion polypeptide comprises SEQ ID NO:95.

97. The method of claim 79, wherein the first fusion polypeptide is SEQ ID NO: 67; and the second fusion polypeptide is SEQ ID NO:95.

98. The method of claim 79, wherein the first fusion polypeptide comprises SEQ ID NO: 67; and the second fusion polypeptide comprises SEQ ID NO:96.

99. The method of claim 79, wherein the first fusion polypeptide is SEQ ID NO: 67; and the second fusion polypeptide is SEQ ID NO:96.

100. The method of claim 79, wherein the first fusion polypeptide comprises SEQ ID NO: 68; and the second fusion polypeptide comprises SEQ ID NO:95.

101. The method of claim 79, wherein the first fusion polypeptide is SEQ ID NO: 68; and the second fusion polypeptide is SEQ ID NO:95.

102. The method of claim 79, wherein the first fusion polypeptide comprises SEQ ID NO: 68; and the second fusion polypeptide comprises SEQ ID NO:96.

103. The method of claim 79, wherein the first fusion polypeptide is SEQ ID NO: 68; and the second fusion polypeptide is SEQ ID NO:96.

104. The method of claim 79, wherein the protein consists of the first fusion polypeptide and the second fusion polypeptide.

105. The method of claim 79, wherein the protein is comprised within a pharmaceutical composition that further comprises a pharmaceutically acceptable excipient.


SEQUENCE LISTING (129 sequences)

SEQ ID NO: 1 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 216 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 4 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 15 aa, Homo sapiens
EPKSCDKTHTCPPCP

SEQ ID NO: 6 - protein, 10 aa, Homo sapiens
DKTHTCPPCP

SEQ ID NO: 7 - skipped

SEQ ID NO: 8 - protein, 5 aa, synthetic construct
  REGION 1..5: note = Description of Artificial Sequence: Synthetic peptide
GGGGS

SEQ ID NO: 9 - protein, 10 aa, synthetic construct
  REGION 1..10: note = Description of Artificial Sequence: Synthetic peptide
GGGGSGGGGS

SEQ ID NO: 10 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 11 - protein, 181 aa, Homo sapiens
NSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVS

SEQ ID NO: 12 - protein, 217 aa, synthetic construct
  REGION 1..217: note = Description of Artificial Sequence: Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 13 - protein, 216 aa, synthetic construct
  REGION 1..216: note = Description of Artificial Sequence: Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 14 - protein, 217 aa, synthetic construct
  REGION 1..217: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 15 - protein, 216 aa, synthetic construct
  REGION 1..216: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 16 - protein, 217 aa, synthetic construct
  REGION 1..217: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 17 - protein, 216 aa, synthetic construct
  REGION 1..216: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 18 - protein, 217 aa, synthetic construct
  REGION 1..217: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 19 - protein, 216 aa, synthetic construct
  REGION 1..216: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 20-23 - skipped (4 entries)

SEQ ID NO: 24 - protein, 217 aa, synthetic construct
  REGION 1..217: note = Description of Artificial Sequence: Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 216 aa, synthetic construct
  REGION 1..216: note = Description of Artificial Sequence: Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 217 aa, synthetic construct
  REGION 1..217: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 216 aa, synthetic construct
  REGION 1..216: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 227 aa, synthetic construct
  REGION 1..227: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 226 aa, synthetic construct
  REGION 1..226: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 30 - protein, 227 aa, synthetic construct
  REGION 1..227: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 31 - protein, 226 aa, synthetic construct
  REGION 1..226: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 32 - protein, 217 aa, synthetic construct
  REGION 1..217: note = Description of Artificial Sequence: Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWANYKTTPPVLDSDGSFFLYSKLTVTKEEWQQGFVFSCSVMHEALHNHYTQKSLSLSPGK

SEQ ID NO: 33 - protein, 216 aa, synthetic construct
  REGION 1..216: note = Description of Artificial Sequence: Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWANYKTTPPVLDSDGSFFLYSKLTVTKEEWQQGFVFSCSVMHEALHNHYTQKSLSLSPG

SEQ ID NO: 34 - protein, 217 aa, synthetic construct
  REGION 1..217: note = Description of Artificial Sequence: Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 35 - protein, 216 aa, synthetic construct
  REGION 1..216: note = Description of Artificial Sequence: Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 36 - protein, 217 aa, synthetic construct
  REGION 1..217: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 37 - protein, 216 aa, synthetic construct
  REGION 1..216: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 38 - protein, 217 aa, synthetic construct
  REGION 1..217: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 39 - protein, 216 aa, synthetic construct
  REGION 1..216: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 40 - protein, 217 aa, synthetic construct
  REGION 1..217: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 41 - protein, 216 aa, synthetic construct
  REGION 1..216: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 42-47 - skipped (6 entries)

SEQ ID NO: 48 - protein, 217 aa, synthetic construct
  REGION 1..217: note = Description of Artificial Sequence: Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 49 - protein, 216 aa, synthetic construct
  REGION 1..216: note = Description of Artificial Sequence: Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPG

SEQ ID NO: 50 - protein, 217 aa, synthetic construct
  REGION 1..217: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 51 - protein, 216 aa, synthetic construct
  REGION 1..216: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPG

SEQ ID NO: 52 - protein, 217 aa, synthetic construct
  REGION 1..217: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 53 - protein, 216 aa, synthetic construct
  REGION 1..216: note = Description of Artificial Sequence: Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPG

SEQ ID NO: 54 - protein, 227 aa, synthetic construct
  REGION 1..227: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 55 - protein, 226 aa, synthetic construct
  REGION 1..226: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 56 - protein, 227 aa, synthetic construct
  REGION 1..227: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 57 - protein, 226 aa, synthetic construct
  REGION 1..226: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 58 - protein, 502 aa, Homo sapiens
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 59 - protein, 482 aa, Homo sapiens
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 60 - protein, 482 aa, Homo sapiens
  SITE 50: note = MOD_RES - Formylglycine residue
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 61 - protein, 714 aa, synthetic construct
  REGION 1..714: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 62 - protein, 713 aa, synthetic construct
  REGION 1..713: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 63 - protein, 714 aa, synthetic construct
  SITE 50: note = MOD_RES - Formylglycine residue
  REGION 1..714: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 64 - protein, 713 aa, synthetic construct
  SITE 50: note = MOD_RES - Formylglycine residue
  REGION 1..713: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 65 - protein, 714 aa, synthetic construct
  REGION 1..714: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 66 - protein, 713 aa, synthetic construct
  REGION 1..713: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 67 - protein, 714 aa, synthetic construct
  SITE 50: note = MOD_RES - Formylglycine residue
  REGION 1..714: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 68 - protein, 713 aa, synthetic construct
  SITE 50: note = MOD_RES - Formylglycine residue
  REGION 1..713: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 69 - protein, 714 aa, synthetic construct
  REGION 1..714: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 713 aa, synthetic construct
  REGION 1..713: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 714 aa, synthetic construct
  SITE 50: note = MOD_RES - Formylglycine residue
  REGION 1..714: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 713 aa, synthetic construct
  SITE 50: note = MOD_RES - Formylglycine residue
  REGION 1..713: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 711 aa, synthetic construct
  REGION 1..711: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 74 - protein, 710 aa, synthetic construct
  REGION 1..710: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 75 - protein, 711 aa, synthetic construct
  SITE 50: note = MOD_RES - Formylglycine residue
  REGION 1..711: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 76 - protein, 710 aa, synthetic construct
  SITE 50: note = MOD_RES - Formylglycine residue
  REGION 1..710: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 77 - protein, 711 aa, synthetic construct
  REGION 1..711: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 710 aa, synthetic construct
  REGION 1..710: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 711 aa, synthetic construct
  SITE 50: note = MOD_RES - Formylglycine residue
  REGION 1..711: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 710 aa, synthetic construct
  SITE 50: note = MOD_RES - Formylglycine residue
  REGION 1..710: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 719 aa, synthetic construct
  REGION 1..719: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 82 - protein, 718 aa, synthetic construct
  REGION 1..718: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 83 - protein, 719 aa, synthetic construct
  SITE 50: note = MOD_RES - Formylglycine residue
  REGION 1..719: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 84 - protein, 718 aa, synthetic construct
  SITE 50: note = MOD_RES - Formylglycine residue
  REGION 1..718: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 85 - protein, 719 aa, synthetic construct
  REGION 1..719: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 718 aa, synthetic construct
  REGION 1..718: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 87 - protein, 719 aa, synthetic construct
  SITE 50: note = MOD_RES - Formylglycine residue
  REGION 1..719: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 718 aa, synthetic construct
  SITE 50: note = MOD_RES - Formylglycine residue
  REGION 1..718: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 714 aa, synthetic construct
  REGION 1..714: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 90 - protein, 713 aa, synthetic construct
  REGION 1..713: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 91 - protein, 714 aa, synthetic construct
  SITE 50: note = MOD_RES - Formylglycine residue
  REGION 1..714: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 92 - protein, 713 aa, synthetic construct
  SITE 50: note = MOD_RES - Formylglycine residue
  REGION 1..713: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 93 - protein, 714 aa, synthetic construct
  REGION 1..714: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 94 - protein, 713 aa, synthetic construct
  REGION 1..713: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 95 - protein, 714 aa, synthetic construct
  SITE 50: note = MOD_RES - Formylglycine residue
  REGION 1..714: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 96 - protein, 713 aa, synthetic construct
  SITE 50: note = MOD_RES - Formylglycine residue
  REGION 1..713: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 97 - protein, 714 aa, synthetic construct
  REGION 1..714: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 98 - protein, 713 aa, synthetic construct
  REGION 1..713: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPG

SEQ ID NO: 99 - protein, 714 aa, synthetic construct
  SITE 50: note = MOD_RES - Formylglycine residue
  REGION 1..714: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 100 - protein, 713 aa, synthetic construct
  SITE 50: note = MOD_RES - Formylglycine residue
  REGION 1..713: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPG

SEQ ID NO: 101 - protein, 711 aa, synthetic construct
  REGION 1..711: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 102 - protein, 710 aa, synthetic construct
  REGION 1..710: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 103 - protein, 711 aa, synthetic construct
  SITE 50: note = MOD_RES - Formylglycine residue
  REGION 1..711: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 104 - protein, 710 aa, synthetic construct
  SITE 50: note = MOD_RES - Formylglycine residue
  REGION 1..710: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 105 - protein, 711 aa, synthetic construct
  REGION 1..711: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 106 - protein, 710 aa, synthetic construct
  REGION 1..710: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPG

SEQ ID NO: 107 - protein, 711 aa, synthetic construct
  SITE 50: note = MOD_RES - Formylglycine residue
  REGION 1..711: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 108 - protein, 710 aa, synthetic construct
  SITE 50: note = MOD_RES - Formylglycine residue
  REGION 1..710: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPG

SEQ ID NO: 109 - protein, 719 aa, synthetic construct
  REGION 1..719: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 110 - protein, 718 aa, synthetic construct
  REGION 1..718: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 111 - protein, 719 aa, synthetic construct
  SITE 50: note = MOD_RES - Formylglycine residue
  REGION 1..719: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 112 - protein, 718 aa, synthetic construct
  SITE 50: note = MOD_RES - Formylglycine residue
  REGION 1..718: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 113 - protein, 719 aa, synthetic construct
  REGION 1..719: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 114 - protein, 718 aa, synthetic construct
  REGION 1..718: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPG

SEQ ID NO: 115 - protein, 719 aa, synthetic construct
  SITE 50: note = MOD_RES - Formylglycine residue
  REGION 1..719: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 116 - protein, 718 aa, synthetic construct
  SITE 50: note = MOD_RES - Formylglycine residue
  REGION 1..718: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPG

SEQ ID NO: 117 - protein, 713 aa, synthetic construct
  REGION 1..713: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSRPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 118 - protein, 713 aa, synthetic construct
  SITE 281: note = MOD_RES - Formylglycine residue
  REGION 1..713: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSRPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 119 - protein, 713 aa, synthetic construct
  REGION 1..713: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGGGGGSRPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 120 - protein, 713 aa, synthetic construct
  SITE 281: note = MOD_RES - Formylglycine residue
  REGION 1..713: note = Description of Artificial Sequence: Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGGGGGSRPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 121 - protein, 19 aa, synthetic construct
  REGION 1..19: note = Description of Artificial Sequence: Synthetic peptide
MGWSCIILFLVATATGAYA

SEQ ID NO: 122 - protein, 714 aa, synthetic construct
  REGION 1..714: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWSSYKTTPPVLDSDGSFFLYSKLTVTKEEWQQGFVFSCSVMHEALHNHYTQKSLSLSPGK

SEQ ID NO: 123 - protein, 713 aa, synthetic construct
  REGION 1..713: note = Description of Artificial Sequence: Synthetic polypeptide
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWSSYKTTPPVLDSDGSFFLYSKLTVTKEEWQQGFVFSCSVMHEALHNHYTQKSLSLSPG

SEQ ID NO: 124 - protein, 714 aa, synthetic construct
  SITE 50: note = MOD_RES - Formylglycine residue
  REGION 1..714: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWSSYKTTPPVLDSDGSFFLYSKLTVTKEEWQQGFVFSCSVMHEALHNHYTQKSLSLSPGK

SEQ ID NO: 125 - protein, 713 aa, synthetic construct
  SITE 50: note = MOD_RES - Formylglycine residue
  REGION 1..713: note = Description of Artificial Sequence: Synthetic polypeptide
RPRNALLLLADDGGFESGAYNNSAIATPHLDALARRSLLFRNAFTSVSSXSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWSSYKTTPPVLDSDGSFFLYSKLTVTKEEWQQGFVFSCSVMHEALHNHYTQKSLSLSPG

SEQ ID NO: 126 - skipped

SEQ ID NO: 127 - protein, 13 aa, synthetic construct
  REGION 1..13: note = Description of Artificial Sequence: Synthetic peptide
NAFTSVSSCSPSR

SEQ ID NO: 128 - protein, 13 aa, synthetic construct
  SITE 9: note = MOD_RES - Alkylated carbamidomethyl Cys
  REGION 1..13: note = Description of Artificial Sequence: Synthetic peptide
NAFTSVSSXSPSR

SEQ ID NO: 129 - protein, 13 aa, synthetic construct
  SITE 9: note = MOD_RES - Formylglycine residue
  REGION 1..13: note = Description of Artificial Sequence: Synthetic peptide
NAFTSVSSXSPSR